(19) United States
(12) Patent Application Publication
(10) Pub. No.: US 2009/0197859 A1
(43) Pub. Date: Aug. 6, 2009

(54) Pyridinyl Amides for the Treatment of CNS and Metabolic Disorders

(75) Inventors: Elizabeth Martha Collantes, Gales Ferry, CT (US); Jacob Bradley Schwarz, Waterford, CT (US)

Correspondence Address: PFIZER INC;Mary J Hosley, 150 EAST 42ND STREET, 5TH FLOOR - STOP 49S, NEW YORK, NY, 10017-5612, US

(73) Assignee: Pfizer Inc.

(21) Appl. No.: 12/365,305

(22) Filed: Feb. 4, 2009

Related U.S. Application Data
(60) Provisional application No. 61/026,195, filed on Feb. 5, 2008.

(51) Int. Cl. A61K 31/496; C07D 401/14; A61K 31/551; A61P 25/18; A61K 31/397; C07D 243/08

(52) U.S. Cl. 514/210.2; 544/364; 540/575; 514/253.01; 514/218

(57) ABSTRACT

The present invention relates to novel pyridinyl derivatives of Formula

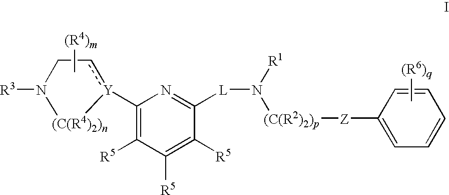

wherein Y, Z, L, R¹ through R¹¹, n, m, p, q, t are as defined herein, that are 5-HT receptor ligands, particularly the 5-HT6 subtype, and as such are useful for treating diseases wherein modulation of 5-HT activity is desired. The present invention relates to novel pyridinyl derivatives including their pharmaceutically acceptable salts. The invention also relates to processes for the preparation of, intermediates used in the preparation of, pharmaceutical compositions containing and the uses of such compounds in treating diseases of the central nervous system such as schizophrenia.

### BACKGROUND OF THE INVENTION

The present invention relates to novel pyridinyl derivatives that are 5-HT receptor ligands, particularly the 5-HT6 subtype, and as such are useful for treating diseases wherein modulation of 5-HT activity is desired. The present invention relates to novel pyridinyl derivatives including their pharmaceutically acceptable salts. The invention also relates to processes for the preparation of, intermediates used in the preparation of, pharmaceutical compositions containing and the uses of such compounds in treating diseases of the central nervous system such as schizophrenia.

Serotonin (5-Hydroxytryptamine)(5-HT) receptors play a critical role in many physiological and behavioral functions in humans and animals. These functions are mediated through various 5-HT receptors distributed throughout the body. There are now approximately fifteen different human 5-HT receptor subtypes that have been cloned, many with well-defined roles in humans. One of the most recently identified 5-HT receptor subtypes is the 5-HT6 receptor, first cloned from rat tissue in 1993 (Monsma, F. J.; Shen, Y.; Ward, R. P.; Hamblin, M. W. Molecular Pharmacology 1993, 43, 320-327) and subsequently from human tissue (Kohen, R.; Metcalf, M. A.; Khan, N.; Druck, T.; Huebner, K.; Sibley, D. R.; Journal of Neurochemistry 1996, 66, 47 56). The receptor is a G-protein coupled receptor (GPCR) positively coupled to adenylate cyclase (Ruat, M.; Traiffort, E.; Arrang, J-M.; Tardivel-Lacombe, L.; Diaz, L.; Leurs, R.; Schwartz, J-C.; Biochemical Biophysical Research Communications 1993, 193, 268-276). The receptor is found almost exclusively in the central nervous system (CNS) areas both in rat and in human. In situ hybridization studies of the 5-HT6 receptor in rat brain using mRNA indicate principal localization in the areas of 5-HT projection including striatum, nucleus accumbens, olfactory tubercle, and hippocampal formation (Ward, R. P.; Hamblin, M. W.; Lachowicz, J. E.; Hoffman, B. J.; Sibley, D. R.; Dorsa, D. M. Neuroscience 1995, 64, 1105 1111).

There are many potential therapeutic uses for 5-HT6 ligands in humans based on direct effects and on indications from available scientific studies. These studies include the localization of the receptor, the affinity of ligands with known in vivo activity, and various animal studies conducted so far.

One potential therapeutic use of modulators of 5-HT6 receptor function is in the enhancement of cognition and memory in human diseases such as Alzheimer's Disease. The high levels of receptor found in important structures in the forebrain, including the caudate/putamen, hippocampus, nucleus accumbens, and cortex suggest a role for the receptor in memory and cognition since these areas are known to play a vital role in memory (Gerard, C.; Martres, M.-P.; Lefevre, K.; Miquel, M. C.; Verge, D.; Lanfumey, R.; Doucet, E.; Hamon, M.; El Mestikawy, S. Brain Research, 1997, 746, 207 219). The ability of known 5-HT6 receptor ligands to enhance cholinergic transmission has added to the recognition of the cognition use (Bentley, J. C.; Boursson, A.; Boess, F. G.; Kone, F. C.; Marsden, C. A.; Petit, N.; Sleight, A. J. British Journal of Pharmacology, 1999, 126(7). 1537-1542). Studies have found that a known 5-HT6 selective antagonist significantly increased glutamate and aspartate levels in the frontal cortex without elevating levels of noradrenaline, dopamine, or 5-HT. This selective elevation of neurochemicals known to be involved in memory and cognition strongly implicate a role for 5-HT6 ligands in cognition (Dawson, L. A.; Nguyen, H. Q.; Li, P. British Journal of Pharmacology, 2000, 130(1), 23 26). Additionally, animal studies of memory and learning with a known selective 5-HT6 antagonist found some positive effects (Rogers, D. C.; Hatcher, P. D.; Hagan, J. J. Society of Neuroscience, Abstracts 2000, 26, 680). Further support for the role of a selective 5-HT6 ligand in cognition can be found in Woolley, M. L.; Marsden, C. A.; Sleight, A. J.; and Fone, K. C. F., Psychopharmacology, 2003, 170(4), 358 367.

A related potential therapeutic use for 5-HT6 ligands is the treatment of attention deficit disorders (ADD, also known as Attention Deficit Hyperactivity Disorder or ADHD) in both children and adults. Because 5-HT6 antagonists appear to enhance the activity of the nigrostriatal dopamine pathway and because ADHD has been linked to abnormalities in the caudate 5-HT6 those skilled in the art expect that antagonists attenuate attention deficit disorders (Ernst, M; Zametkin, A. J.; Matochik, J. H.; Jons, P. A.; Cohen, R. M. Journal of Neuroscience 1998, 18(15), 5901 5907).

Early studies examining the affinity of various CNS ligands with known therapeutic utility or a strong structural resemblance to known drugs suggests a role for 5-HT6 ligands in the treatment of schizophrenia and depression. For example, clozapine and olanzapine (effective clinical antipsychotics) have high affinity for the 5-HT6 receptor subtype. Also, several clinical antidepressants have high affinity for the receptor as well and act as antagonists at this site (Branchek, T. A.; Blackburn, T. P. Annual Reviews in Pharmacology and Toxicology 2000, 40, 319 334).

5-HT6 receptor antagonists are also known to be useful in the treatment of obesity, Drug Discovery Today, 11 (7/8) 2006.

International Patent Publication WO 03/086398, published Oct. 23, 2003 refers to certain aminocarbonyl pyridines and states that they have angiogenesis inhibitors. The compounds of the present invention have not been observed to possess typical antiangiogenic effects such as ovarian follicular atrophy at chronic high dosing.

### SUMMARY OF THE INVENTION

The present invention relates to novel compounds of the Formula

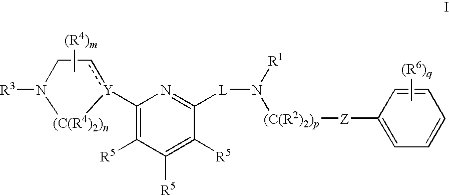

or pharmaceutically acceptable salts thereof wherein,

L is >C═O, or —SO₂—;

Y is >C(R⁷)— and the dashed line (- - -) is absent; or Y is carbon and the dashed line (- - -) is a double bond; or Y is >N— and the dashed line (- - -) is absent;

Z is a bond, —(C(R²)₂)—, —O—, >C═O, or —S(O)ₜ—; wherein t is an integer selected from 0, 1, or 2;

R¹ is selected from the group consisting of hydrogen, —CF₃, (C₁-C₆)alkyl-, (C₂-C₆)alkenyl-, and (C₂-C₆)alkynyl-; wherein each of aforesaid (C₁-C₆)alkyl-, (C₂-C₆)alkenyl-, and (C₂-C₆)alkynyl- may be optionally substituted by one, two, or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₁-C₉)heterocycloalkyl-, (C₆-C₁₀)aryl-, and (C₁-C₉)heteroaryl-;

each R² is independently selected from the radicals consisting of hydrogen, halo, —CF₃, —CN, —NO₂, —(C═O)R⁸, —(C═O)OR⁹, —O(C═O)R⁸, —OR⁹, —NR¹⁰R¹⁰, —SR¹¹, —(S═O)R¹¹, —SO₂R¹¹, (C₁-C₆)alkyl-, (C₂-C₆)alkenyl-, (C₂-C₆)alkynyl-, (C₃-C₁₀)cycloalkyl-, (C₅-C₁₀)cycloalkenyl-, (C₆-C₁₀)bicycloalkyl-, (C₁-C₉)heterocycloalkyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₁-C₉)heterocycloalkyl-, (C₆-C₁₀)aryl-, and (C₁-C₉)heteroaryl-;

or optionally R¹ and one of said R² may optionally be taken together with the carbon or nitrogen to which they are attached to form an optionally substituted 3 to 10 membered heterocyclic ring optionally containing one or two double or triple bonds and optionally containing one or two additional heteroatoms selected from N, S and O;

or optionally two of said R² may optionally be taken together with the carbon to which they are attached to form an optionally substituted 3 to 10 membered carbocyclic ring optionally containing one or two double or triple bonds; and wherein said 3 to 10 membered carbocyclic ring may optionally contain 1, 2 or 3 heteroatoms independently selected from N, S and O;

R³ is selected from the radicals consisting of hydrogen, —CF₃, —SO₂R¹¹, (C₁-C₈)alkyl-, (C₂-C₆)alkenyl-, (C₂-C₆)alkynyl-, (C₃-C₁₀)cycloalkyl-, (C₅-C₁₀)cycloalkenyl-, (C₆-C₁₀)bicycloalkyl-, (C₆-C₁₀)bicycloalkenyl-, (C₁-C₉)heterocycloalkyl-, (C₂-C₉)heterocycloalkenyl-, (C₂-C₉)heterobicycloalkyl-, (C₂-C₉)heterobicycloalkenyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₁-C₉)heterocycloalkyl-, (C₆-C₁₀)aryl-, and (C₁-C₉)heteroaryl-;

each R⁴, R⁵ and R⁶ is independently selected from the radicals consisting of hydrogen, halo, —CF₃, —CN, —NO₂, —(C═O)R⁸, —(C═O)OR⁹, —O(C═O)R⁸, —OCF₃, —OR⁹, —NR¹⁰R¹⁰, —(NR¹⁰)(C═O)R⁸, —SR¹¹, —(S═O)R¹¹, —SO₂R¹¹, (C₁-C₆)alkyl-, (C₂-C₆)alkenyl-, (C₂-C₆)alkynyl-, (C₃-C₁₀)cycloalkyl-, (C₅-C₁₀)cycloalkenyl-, (C₆-C₁₀)bicycloalkyl-, (C₆-C₁₀)bicycloalkenyl-, (C₁-C₉)heterocycloalkyl-, (C₂-C₉)heterocycloalkenyl-, (C₂-C₉)heterobicycloalkyl-, (C₂-C₉)heterobicycloalkenyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₁-C₉)heterocycloalkyl-, (C₆-C₁₀)aryl-, and (C₁-C₉)heteroaryl-;

R⁷ is hydrogen, halo, —OR⁹ or (C₁-C₆)alkyl-;

each R⁸ is independently selected from the radicals consisting of hydrogen, (C₁-C₆)alkyl-, (C₁-C₆)alkenyl-, (C₁-C₆)alkynyl-, (C₃-C₁₀)cycloalkyl-, (C₁-C₉)heterocycloalkyl-, (C₆-C₁₀)aryl-, and (C₁-C₉)heteroaryl-; wherein each of the aforesaid radicals may be optionally substituted by one, two or three substituents independently selected from the group consisting of hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₆-C₁₀)aryl-, and (C₁-C₉)heteroaryl-;

each R⁹ is independently selected from the radicals consisting of hydrogen, (C₁-C₆)alkyl-, (C₂-C₆)alkenyl-, (C₂-C₆)alkynyl-, (C₃-C₁₀)cycloalkyl-, (C₁-C₉)heterocycloalkyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₆-C₁₀)aryl, and (C₁-C₉)heteroaryl-;

each R¹⁰ is independently selected from the radicals consisting of hydrogen, —SO₂—(C₁-C₆)alkyl, (C₁-C₆)alkyl-, (C₂-C₆)alkenyl-, (C₂-C₆)alkynyl-, (C₃-C₁₀)cycloalkyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, (C₁-C₆)alkyl-, (C₁-C₆)alkoxy-, (C₃-C₁₀)cycloalkyl-, (C₅-C₁₀)cycloalkenyl-, (C₆-C₁₀)aryl-, and (C₁-C₉)heteroaryl-;

each R¹¹ is independently selected from the group consisting of hydrogen, amino, (C₁-C₆)alkylamino-, di((C₁-C₆)alkyl)amino-, —CF₃, (C₁-C₆)alkyl-, (C₃-C₁₀)cycloalkyl-, (C₁-C₉)heterocycloalkyl-, (C₆-C₁₀)aryl-, and (C₁-C₉)heteroaryl-;

n is an integer selected from one, two or three;

m an integer selected from zero, one, two, three or four;

p is an integer selected from zero, one, two, three or four; and

q is an integer selected from zero, one, two, three or four.

As used herein, the term “(C₁-C₆)alkyl” is defined to include saturated aliphatic hydrocarbons including straight chains and branched chains. The alkyl group has 1 to 6 carbon atoms. More preferably, the alkyl group has 1 to 4 carbon atoms. Most preferably, it is a lower alkyl having 1 to 3 carbon atoms. For example, as used herein, the term “(C₁-C₆)alkyl,” as well as the alkyl moieties of other groups referred to herein (e.g., (C₁-C₆)alkoxy), refers to linear or branched radicals of 1 to 6 carbon atoms (e.g., methyl, ethyl, n-propyl, isopropyl, n-butyl, iso-butyl, secondary-butyl, tertiary-butyl), optionally substituted by 1 to 5 suitable substituents.

Whenever a numerical range is used in this application, for example when 1 to 6 is used in the definition of “alkyl” means that the alkyl group may contain 1 carbon atom, 2 carbon atoms, 3 carbon atoms, etc. up to and including 6 carbon atoms.

As used herein, the term “(C₂-C₆)alkenyl” is defined to include aliphatic hydrocarbons having at least one carbon-carbon double bond, including straight chains and branched chains having at least one carbon-carbon double bond. The alkenyl group has 2 to 6 carbon atoms. More preferably, the alkenyl group has 2 to 4 carbon atoms. Most preferably, the alkenyl group has 3 to 4 carbon atoms. For example, as used herein, the term “(C₂-C₆)alkenyl” means straight or branched chain unsaturated radicals of 2 to 6 carbon atoms, including, but not limited to ethenyl, 1-propenyl, 2-propenyl(allyl), iso-propenyl, 2-methyl-1-propenyl, 1-butenyl, 2-butenyl, and the like; optionally substituted by 1 to 5 suitable substituents. When the compounds of Formula I contain an alkenyl group, the alkenyl group may exist as the pure E (entgegen) form, the pure Z (zusammen) form, or any mixture thereof.

As used herein, the term “(C₂-C₆)alkynyl” is defined to include aliphatic hydrocarbons having at least one carbon-carbon triple bond, including straight chains and branched chains having at least one carbon-carbon triple bond optionally substituted by 1 to 5 suitable substituents. Preferably, the alkynyl group has 2 to 6 carbon atoms. More preferably, the alkynyl group has 3 to 4 carbon atoms.

As used herein, the term “(C₃-C₁₀)cycloalkyl” is defined to include saturated monocyclic hydrocarbon rings (e.g., cyclopropyl, cyclobutyl, cyclopentyl, cyclohexyl, cycloheptyl, cyclooctyl, cyclononyl); optionally substituted by 1 to 5 suitable substituents. The cycloalkyl group has 3 to 10 carbon atoms. More preferably, the cycloalkyl group has 4 to 8 carbon atoms. Most preferably, the cycloalkyl group has 5 to 6 carbon atoms.

As used herein, the term “(C₃-C₁₀)cycloalkenyl” is defined to include unsaturated (non aromatic) monocyclic hydrocarbon rings (e.g., cyclopropenyl, cyclobutenyl, cyclopentenyl, cyclohexenyl, cycloheptenyl, cyclooctenyl, cyclononenyl. 1,3-cyclobutadiennyl, 1,3 or 1,4 cyclopentadienyl, 1,3 or 1,4 or 1,5 cyclohexadienyl, cycloheptenyl, cyclooctenyl, cyclononenyl); optionally substituted by 1 to 5 suitable substituents. The cycloalkenyl group has 3 to 10 carbon atoms. More preferably, the cycloalkyl group has 4 to 7 carbon atoms. Most preferably, the cycloalkyl group has 5 to 6 carbon atoms. In one embodiment the cycloalkenyl may optionally contain two or more non cumulative non aromatic double bonds.

As used herein, the term “(C₆-C₁₀)bicycloalkyl” is defined to include a 6 to 9 carbon atom cycloalkyl as defined above which is bridged to a second carbocyclic ring (e.g., decahydro naphthalenyl, octahydro indenyl, bicyclo[2.2.1]heptanyl, bicyclo[3.2.1]octanyl and bicyclo[5.2.0]nonanyl, etc.). Preferably, the bicycloalkyl group has 6 to 20 carbon atoms. More preferably, the bicycloalkyl group has 6 to 15 carbon atoms. Most preferably, the bicycloalkyl group has 6 to 12 carbon atoms. The bicycloalkyl is optionally substituted by 1 to 5 suitable substituents.

As used herein, the term “(C₆-C₁₀)bicycloalkenyl” is defined to include a 6 to 10 carbon atom (C₆-C₁₀)bicycloalkyl as defined above which contains one to three non cumulative non aromatic double bonds.

As used herein, the term “(C₆-C₁₀)aryl” is defined to include all-carbon monocyclic or fused-ring polycyclic (i.e., rings which share adjacent pairs of carbon atoms) groups having a completely conjugated pi-electron system. The aryl group has 6, 8, 9 or 10 carbon atoms in the ring(s). Preferably, the aryl group has 6, 8, or 10 carbon atoms in the ring(s). More preferably, the aryl group has 6 or 10 carbon atoms in the ring(s). Most preferably, the aryl group has 6 carbon atoms in the ring(s). For example, as used herein, the term “(C₆-C₁₀)aryl” means aromatic radicals containing from 6 to 10 carbon atoms such as phenyl, naphthyl, tetrahydronaphthyl, anthracenyl, indanyl and the like. The aryl group is optionally substituted by 1 to 5 suitable substituents.

As used herein, the term “(C₁-C₉)heteroaryl” is defined to include monocyclic or fused-ring polycyclic aromatic heterocyclic groups with one to five heteroatoms independently selected from O, S and N in the ring. The heteroaryl group has 5 to 12 ring atoms including one to nine carbon atoms. Preferably, the heteroaryl group has 5 to 10 ring atoms including one to four heteroatoms. More preferably, the heteroaryl group has 5 to 8 ring atoms including one, two or three heteroatoms. Most preferably, the heteroaryl group has 6 to 8 ring atoms including one or two heteroatoms. For example, as used herein, the term “(C₁-C₉) heteroaryl” means aromatic radicals containing at least one ring heteroatom selected from O, S and N and from 1 to 9 carbon atoms such as pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like. The heteroaryl group is optionally substituted by 1 to 5 suitable substituents.

As used herein, the term “(C₁-C₉)heterocycloalkyl” is defined to include a monocyclic, bridged, polycyclic or fused polycyclic saturated 3 to 9 membered ring including 1 to 8 carbon atoms; and 1 to 4 heteroatoms independently selected from O, S and N. Examples of such heterocycloalkyl rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, and the like. Further examples of said heterocycloalkyl rings are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2 pyrazolidin-2-yl, 1,3-pyrazolidin-1-yl, 1,2-tetrahydrothiazin-2-yl, 1,3 tetrahydrothiazin-3-yl, 1,2-tetrahydrodiazin-2-yl, 1,3 tetrahydrodiazin-1-yl, 1,4-oxazin-2-yl, 1,2,5-oxathiazin4-yl and the like. The heterocycloalkyl ring is optionally substituted by 1 to 5 suitable substituents.

As used herein, the term “(C₂-C₉)heterocycloalkenyl-” refers to the aforementioned heterocycloalkyl rings containing 1 to 3 non cumulative non aromatic double bonds.

As used herein, the term “(C₂-C₉)heterobicycloalkyl-” is defined to include a 2 to 9 carbon membered (C₂-C₉)heterocycloalkyl as defined above which is bridged to a second carbocyclic or heterocyclic ring (e.g., decahydroisoquinolinyl, octahydroindolinyl, bicyclo[2.2.1]heptanyl, bicyclo[3.2.1]octanyl and bicyclo[5.2.0]nonanyl, etc.).

As used herein, the term “(C₂-C₉)heterobicycloalkenyl-” is defined to include a 2 to 9 carbon membered (C₂-C₉)heterobicycloalkyl as defined above containing 1 to 3 non cumulative non aromatic double bonds.

The compounds of Formula I may exist in the form of pharmaceutically acceptable salts such as, e.g., acid addition salts and base addition salts of the compounds of Formula I. The phrase “pharmaceutically acceptable salt(s)”, as used herein, unless otherwise indicated, includes salts of acidic or basic groups which may be present in the compounds of Formula I.

Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edislyate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, α-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phospate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The compounds of Formula I that include a basic moiety, such as an amino group, may form pharmaceutically acceptable salts with various amino acids, in addition to the acids mentioned above.

The invention also relates to base addition salts of the compounds of Formula I. The chemical bases that may be used as reagents to prepare pharmaceutically acceptable base salts of the compounds of Formula I that are acidic in nature are those that form non-toxic base salts with such compounds. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

As used herein the terms ““Formula I” and “Formula I or a pharmaceutically acceptable salt thereof” are defined to include all forms of the compound of Formula I, including hydrates, solvates, isomers, crystalline and non-crystalline forms, isomorphs, polymorphs, and metabolites thereof.

The compounds of Formula I, or a pharmaceutically acceptable salt thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. The term ‘solvate’ is used herein to describe a molecular complex comprising the compound of the invention and one or more pharmaceutically acceptable solvent molecules (in stoichiometric or non-stoichiometric amounts, for example, ethanol). The term ‘hydrate’ is employed when said solvent is water. Pharmaceutically acceptable solvates include hydrates and other solvates wherein the solvent of crystallization may be isotopically substituted, e.g. D₂O, d6-acetone, d6-DMSO.

The compounds of Formula I may exist as clathrates or other complexes Included within the scope of the invention are complexes such as clathrates, drug-host inclusion complexes wherein, in contrast to the aforementioned solvates, the drug and host are present in stoichiometric or non-stoichiometric amounts. Also included are complexes of the Formula I containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

Also included within the scope of the invention are metabolites of compounds of Formula I, that is, compounds formed in vivo upon administration of the drug.

Also included within the scope of the invention are polymorphs which as used herein refers to alternate crystallization forms of the same substance.

Compounds of Formula I containing one or more asymmetric carbon atoms can exist as two or more stereoisomers. Where a compound of Formula I contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of Formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

The present invention includes all pharmaceutically acceptable isotopically-labelled compounds of formula I wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as 123I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula 1, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. (- - -)

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagents in place of the non-labelled reagent previously employed.

One specific embodiment of the present invention relates to compounds of Formula I wherein, Y is —C(R⁷)— and the dashed line (- - -) is absent.

Another specific embodiment of the present invention relates to compounds of Formula I wherein, Y is >C(R⁷)—, the dashed line (- - -) is absent and R⁷ is hydrogen.

Another specific embodiment of the present invention relates to compounds of Formula I wherein, Y is >C(R⁷)—, the dashed line (- - -) is absent and R⁷ is halo.

Another specific embodiment of the present invention relates to compounds of Formula I wherein, Y is >C(R⁷)—, the dashed line (- - -) is absent and R⁷is fluoro.

Another specific embodiment of the present invention relates to compounds of Formula I wherein, Y is >C(R⁷)—, the dashed line (- - -) is absent and R⁷ is (C₁-C₆)alkyl.

Another specific embodiment of the present invention relates to compounds of Formula I wherein, Y is >C(R⁷)—, the dashed line (- - -) is absent and R⁷ is methyl or ethyl.

Another specific embodiment of the present invention relates to compounds of Formula I wherein Y is >C(R⁷)—, the dashed line (- - -) is absent and R⁷ is —OR⁹.

Another specific embodiment of the present invention relates to compounds of Formula I wherein R⁹ is H, methyl or CF₃.

Another specific embodiment of the present invention relates to compounds of Formula I wherein, Y is >N— and the dashed line (- - -) is absent.

Another specific embodiment of the present invention relates to compounds wherein n is one. Applicants also herein contemplate other specific embodiments of Formula I, wherein each of the foregoing embodiments of Formula I (i.e. each of the >C(R⁷)— and >N— embodiments hereinafter referred to as the “carbon or nitrogen-based embodiments”) also have n equal to one.

Another specific embodiment of the present invention relates to compounds wherein n is two. Applicants also herein contemplate other specific embodiments of Formula I, wherein each of the foregoing carbon or nitrogen-based embodiments also have n equal to two.

Another specific embodiment of the present invention relates to compounds wherein n is three. Applicants also herein contemplate other specific embodiments of Formula I, wherein each of the foregoing carbon or nitrogen-based embodiments also have n equal to three.

Other specific embodiments of the present invention relate to compounds of Formula I wherein m is zero. Other more specific embodiments of the present invention relate to compounds of Formula I, wherein each of the foregoing carbon or nitrogen based embodiments, also have m as equal to zero.

Yet other specific embodiments of the present invention relate to compounds of Formula I wherein m is one. Even more specific embodiments of the present invention relate to compounds of Formula I wherein each of the foregoing carbon or nitrogen based embodiments, also have m as equal to one.

Applicants also contemplate other specific embodiments of the present invention of compounds of Formula I, wherein m is two and envision other more specific embodiments of the present invention relating to compounds of Formula I, wherein each of the foregoing carbon or nitrogen based embodiments, also have m as equal to two.

An embodiment of particular interest to the present inventors includes compounds of Formula I wherein R³ is hydrogen. These exposed secondary amine rings have distinct properties such that the inventors contemplate individual embodiments with each of the foregoing embodiments of Formula I, wherein Y is either the carbon or nitrogen based embodiments and/or m is embodied as zero, one, two, three or four.

Another embodiment of particular interest to the present inventors includes compounds of Formula I wherein R³ is (C₁-C₆)alkyl (more particularly when said alkyl is either methyl, ethyl or propyl, although methyl is of more particular interest) and more particularly wherein this alkyl cap is optionally substituted with by one, two or three substituents independently selected from the group consisting of hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₂-C₉)heterocycloalkyl-, (C₆-C₁₀)aryl-, and (C₁-C₉)heteroaryl-. These alkyl capped secondary amine rings also have distinct properties such that the inventors contemplate individual embodiments with each of the foregoing embodiments of Formula I, wherein Y is either the carbon or nitrogen based embodiments and/or m is embodied as zero, one, two, three or four and/or n is one, two or three.

Another embodiment of particular interest to the present inventors includes compounds of Formula I, wherein R³ is optionally substituted (C₂-C₆)alkenyl-, or (C₂-C₆)alkynyl-. These vinyl and alkynyl capped secondary amine rings are of interest to the inventors and thus they contemplate individual embodiments with each of the foregoing embodiments of Formula I, wherein Y is either the carbon or nitrogen based embodiments and/or m is embodied as zero, one, two, three or four and/or n is one, two or three.

Another embodiment of particular interest to the present inventors includes compounds of Formula I, wherein R³ is —CF₃, —SR¹¹, —(S═O)R¹¹, or —SO₂R¹¹ . These trifluoro and sulfur based capped secondary amine rings are of interest to the inventors and thus they contemplate individual embodiments with each of the foregoing embodiments of Formula I, wherein Y is either the carbon or nitrogen based embodiments and/or m is embodied as zero, one, two, three or four and/or n is one, two or three.

Another embodiment of particular interest to the present inventors includes compounds of Formula I, wherein R³ is (C₃-C₁₀)cycloalkyl- or (C₅-C₁₀)cycloalkenyl-; wherein each of the aforesaid radicals may be optionally substituted by one, two or three substituents independently selected from the group consisting of hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₂-C₆)alkenyl-, (C₂-C₆)alkynyl-, (C₃-C₁₀)cycloalkyl-, (C₅-C₁₀)cycloalkenyl-, (C₆-C₁₀)aryl-, and (C₁-C₉)heteroaryl-. These ring systems are also of interest to the inventors and thus they contemplate individual embodiments with each of the foregoing embodiments of Formula I, wherein Y is either the carbon or nitrogen based embodiments and/or m is embodied as zero, one, two, three or four and/or n is one, two or three.

Another embodiment of particular interest to the present inventors includes compounds of Formula I, wherein R³ is (C₆-C₁₀)bicycloalkyl-, (C₆-C₁₀)bicycloalkenyl-, (C₁-C₉)heterocycloalkyl-, (C₂-C₉)heterocycloalkenyl-, (C₂-C₉)heterobicycloalkyl-, (C₂-C₉)heterobicycloalkenyl-, (C₆-C₁₀)aryl-, or (C₁-C₉)heteroaryl-; wherein each of the aforesaid radicals may be optionally substituted by one, two or three substituents independently selected from the group consisting of hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₂-C₆)alkenyl-, (C₂-C₆)alkynyl-, (C₃-C₁₀)cycloalkyl-, (C₅-C₁₀)cycloalkenyl-, (C₆-C₁₀)aryl-, and (C₁-C₉)heteroaryl-. These polycyclic ring systems are of interest to the inventors and thus they contemplate individual embodiments with each of the foregoing embodiments of Formula I, wherein Y is either the carbon or nitrogen based embodiments and/or m is embodied as zero, one, two, three or four and/or n is one, two or three.

Another embodiment of particular interest to the present inventors includes compounds of Formula I, wherein L is >C═O. These heteroaryl amide ring systems (i.e. L taken together with NR¹) are of particular preferred interest to the inventors and thus they contemplate specific individual embodiments with each of the foregoing embodiments of Formula I, wherein Y is either the carbon or nitrogen based embodiments, m is embodied as zero, one, two, three or four, and/or n is one, two or three; and R³ is embodied as one of the distinct capped embodiments described above. These embodiments particularly include subgeneric embodiments of combinations of each of these features, including either of the Y based carbon or nitrogen embodiments with any of the “m” embodiments in combination with any of the “n” embodiments in combination with any of the R³ embodiments. Rather than reciting this understanding hereinbelow, one skilled in the art will understand that the present inventors fully contemplate such combinations and sub-combinations with each stated embodiment.

Another embodiment of particular interest to the present inventors includes compounds of Formula I wherein L is —SO₂—. These heteroaryl sulfonamide ring systems (i.e. L taken together with NR¹) are of particular interest to the inventors and thus they contemplate individual embodiments with each of the foregoing embodiments of Formula I, wherein Y is either the carbon or nitrogen based embodiments, m is embodied as zero, one, two, three or four, and/or n is one, two or three and R³ is embodied as one of the distinct capped embodiments described above.

Another embodiment of interest to the present inventors includes compounds of Formula I wherein q is 1 or 2 and each R⁶ is independently selected from the radicals consisting of halo, —CF₃, —CN, —NO₂, —(C═O)R⁸, —(C═O)OR⁹, —O(C═O)R⁸, —OCF₃, —OR⁹, —O(C═O)OR⁹, —NR¹⁰R¹⁰, —(NR¹⁰)(C═O)R⁸, —SR¹¹, —(S═O)R¹¹, —SO₂R¹¹, (C₁-C₆)alkyl-, (C₂-C₆)alkenyl-, (C₂-C₆)alkynyl-, (C₃-C₁₀)cycloalkyl-, (C₆-C₁₀)aryl-, and (C₁-C₉)heteroaryl-; wherein each of the aforesaid radicals may be optionally substituted by one, two or three substituents independently selected from the group consisting of hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₂-C₆)alkenyl-, (C₂-C₆)alkynyl-, (C₃-C₁₀)cycloalkyl-, (C₅-C₁₀)cycloalkenyl-, (C₆-C₁₀)bicycloalkyl-, (C₆-C₁₀)bicycloalkenyl-, (C₁-C₉)heterocycloalkyl-, (C₂-C₉)heterocycloalkenyl-, (C₂-C₉)heterobicycloalkyl-, (C₂-C₉)heterobicycloalkenyl-, (C₆-C₁₀)aryl-, and (C₁-C₉)heteroaryl-.

Another embodiment of particular interest to the present inventors includes compounds of Formula I wherein Z is a bond. Compounds of even more particular interest, wherein Z is a bond, include those compounds wherein at least one R² is other than hydrogen or those compounds wherein each R² is hydrogen. These aryl substituted cyclic or acyclic amides are of particular interest to the inventors and thus they contemplate individual embodiments with each of the foregoing embodiments of Formula I, wherein Y is either the carbon or nitrogen based embodiments, m is embodied as zero, one, two or three; n is one, two or three; and R³ is embodied as one of the distinct capped embodiments described above.

Another embodiment of particular interest to the present inventors includes compounds of Formula I wherein Z is —(C(R²)₂)—. Compounds of even more particular interest, wherein Z is —(C(R²)₂)—, include those compounds wherein at least one R² is other than hydrogen or those compounds wherein each R² is hydrogen. These tolyl substituted cyclic or acyclic amides are of particular interest to the inventors and thus they contemplate individual embodiments with each of the foregoing embodiments of Formula I, wherein Y is either the carbon or nitrogen based embodiments, m is embodied as zero, one, two, three or four; n is one, two or three; and R³ is embodied as one of the distinct capped embodiments described above.

Another embodiment of particular interest to the present inventors includes compounds of Formula I, wherein Z is —O—. These aryloxy-cyclic or acyclic amides are of particular interest to the inventors and thus they contemplate individual embodiments with each of the foregoing embodiments of Formula I, wherein Y is either the carbon or nitrogen based embodiments, m is embodied as zero, one or two; n is one, two or three; and R³ is embodied as one of the distinct capped embodiments described above.

Another embodiment of particular interest to the present inventors includes compounds of Formula I wherein Z is >C═O. These aroyl substituted cyclic or acyclic amides are of particular interest to the inventors and thus they contemplate individual embodiments with each of the foregoing embodiments of Formula I, wherein Y is either the carbon or nitrogen based embodiments, m is embodied as zero, one or two; n is one, two or three; and R³ is embodied as one of the distinct capped embodiments described above.

Another embodiment of particular interest to the present inventors includes compounds of Formula I wherein Z is or —S(O)ₜ—; wherein t is an integer selected from 0, 1, or 2. These aryl sulfonyl cyclic or acyclic amides are of particular interest to the inventors and thus they contemplate individual embodiments with each of the foregoing embodiments of Formula I, wherein Y is either the carbon or nitrogen based embodiments, m is embodied as zero, one or two; n is one, two or three; and R³ is embodied as one of the distinct capped embodiments described above.

Another embodiment of particular preferred interest to the present inventors includes compounds of Formula I wherein R¹ and one of said R² may optionally be taken together with the carbon or nitrogen to which they are attached to form an optionally substituted 3 to 10 membered heterocyclic ring optionally containing one or two double or triple bonds and optionally containing one or two additional heteroatoms selected from N, S and O. Such compounds, described as formula

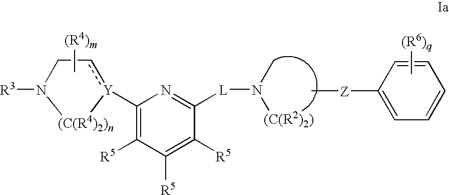

contain a —N—(C(R²)₂)— ring of 3 to 10 ring members These azacyclic compounds are of particular preferred interest to the inventors and thus they contemplate individual embodiments with each of the foregoing embodiments of Formula I, wherein Y is either the carbon or nitrogen based embodiments, m is embodied as zero, one or two; n is one, two or three; and R³ is embodied as one of the distinct capped embodiments described above. A more specific embodiment of these azacyclic compounds of particular interest to the present inventors includes compounds of Formula I, wherein said optionally substituted 3 to 10 membered heterocyclic ring optionally contains one or two double or triple bonds, is selected from the group consisting of azetidinyl, pyrrolidinyl, 3-pyrolin-1-yl, piperidinyl, 1,2,3,6-tetrahydropyridin-1-yl, perhydroazepinyl, heptamethyleneinyl, octahydroazoninyl, azabicyclo(2.2.1)heptan-3-one, tropanyl (azabicyclo[3.2.1]octane); and more specifically (1,4)-piperidinyl, (1,3)-piperidinyl, and (1,3)-pyrrolidinyl.

Another embodiment of particular interest to the present inventors includes compounds of Formula I wherein two of said R² groups may optionally be taken together with the carbon to which they are attached to form an optionally substituted 3 to 10 membered carbocyclic ring optionally containing one or two double or triple bonds; and wherein said 3 to 10 membered carbocyclic ring may optionally contain 1, 2 or 3 heteroatoms. Such compounds, described as formula

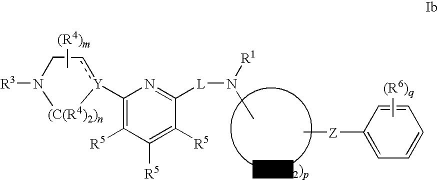

wherein the —(C(R²)₂)ₚ— ring contains 3 to 10 ring members. These carbocyclic or heterocyclic (wherein one of the carbon atoms is replaced by a nitrogen, oxygen or sulfur heteroatom) compounds are of particular interest to the inventors and thus they contemplate individual embodiments with each of the foregoing embodiments of Formula I, wherein Y is either the carbon or nitrogen based embodiments, m is embodied as zero, one or two; n is one, two or three; and R³ is embodied as one of the distinct capped embodiments described above. A more specific embodiment of these carbocyclic or heterocyclic compounds of particular interest to the present inventors includes compounds of Formula I wherein said optionally substituted 3 to 10 membered heterocyclic ring optionally containing one or two double or triple bonds is selected from the group consisting of cyclopropyl, cyclobutyl, cyclobutenyl, cyclopentyl, cyclopentenyl, cyclohexyl, cyclohexenyl, cyclohexadinenyl, azetidinyl, pyrolidinyl, or piperidinyl; more specifically (2,4)-cyclohexadinenyl, (2,5)-cyclohexadinenyl, (1,4)-piperidinyl, (1,3)-piperidinyl, or (1,3)-pyrrolidinyl.

The present inventors also herein contemplate the following individual species exemplifying certain of the aforementioned embodiments:

(3,5-dichloro-6-(1-(furan-2-yl)-2-methylpiperidin-4-yl)pyridin-2-yl)(3-(3-chlorophenyl)pyrrolidin-1-yl)methanone;

[3-(3-chloro-phenyl)-pyrrolidin-1-yl]-(3,5-dichloro-1′-furan-2-yl-2′-methyl-1′,2′,3′,4′,5′,6′hexahydro-[2,4′]bipyridinyl-6yl)methanone;

N-(4-methoxyphenethyl)-5-chloro-6-(5-(trifluoromethyl)-2-methyl(1-methyl-1H-pyrrol-3-yl)piperazin-1-yl)-N-methylpyridine-2-carboxamide;

5-chloro-6-[2-methyl-4-(1H-pyrrol-3-yl)-5-trifluoromethyl-piperazin-1-yl]-pyridine-2-carboxylic acid [2-(4-methoxy-phenyl)-ethyl]-methyl-amide;

(4-chloro-1′-methanesulfonyl-1′,2′,3′,4′,5′,6′-hexahydro-[2,4′]bipyridinyl-6-yl)-[4-(3-methyl-benzyl)-piperidin-1-yl]-methanone;

[3,4-dimethoxy-1′-(1-methyl-allyl)-1′,2′,3′,4′,5′,6′-hexahydro-[2,4′]bipyridinyl-6-yl]-[4-(3-methoxy-benzyl)-piperidin-1-yl]-methanone;

(4-(m-tolylsulfonyl)piperidin-1-yl)(6-(4-methylpiperazin-1-yl)pyridin-2-yl)methanone;

[6-(4-methyl-piperazin-1-yl)-pyridin-2-yl]-[4-(toluene-3-sulfonyl)-piperidin-1-yl]-methanone;

N-(2-(3-(trifluoromethyl)phenoxy)ethyl-6-(5-(trifluoromethyl )-2-methyl-4-(1-methylpyrrolidin-3-yl)piperazin-1-yl )-N-methylpyridine-2-carboxamide;

6-[2-methyl-4-(1-methyl-pyrrolidin-3-yl)-5-trifluoromethyl-piperazin-1-yl]-pyridine-2-carboxylic acid methyl-[2-(3-trifluoromethyl-phenoxy)-ethyl]-amide;

(4-(m-tolyloxy)piperidin-1-yl )(6-(4-fluoro-1-methylpiperidin-4-yl )pyridin-2-yl)methanone;

(4′-fluoro-1′-methyl-1′,2′,3′,4′,5′,6′-hexahydro-[2,4′]bipyridinyl-6-yl)-(4-m-tolyloxy-piperidin-1-yl)-methanone;

3-[1-(1′-isopropyl-4′-methyl-1′,2′,3′,4′,5′,6′-hexahydro-[2,4′]bipyridinyl-6-carbonyl)-piperidin-4-yloxy]-benzonitrile;

6-(3-phenoxy-pyrrolidine-1-sulfonyl)-1′-trifluoromethyl-2′,3′,5′,6′-tetrahydro-1′H-[2,4′]bipyridinyl-4′-ol;

6-(2,2,4-trimethylpiperazin-1-yl)-N-(2-phenoxyethyl)pyridine-2-carboxamide;

6-(2,2,4-trimethyl-piperazin-1-yl)-pyridine-2-carboxylic acid (2-phenoxy-ethyl)-amide;

6-(3,4,5-trimethylpiperazin-1-yl)-N-(2-phenoxyethyl)pyridine-2-carboxamide; or

6-(3,4,5-trimethyl-piperazin-1-yl)-pyridine-2-carboxylic acid (2-phenoxy-ethyl)-amide;

or pharmaceutically acceptable salts thereof.

In another embodiment, the invention also relates to the compounds described as Examples 1-101 in the Examples section of the subject application, and pharmaceutically acceptable salts (including solvates and hydrates) thereof.

Specific piperazine embodiments of the invention also include:

(6-Piperazin-1-yl)-pyridin-2-yl)-(4-o-tolyl-piperidin-1-yl)-methanone;

[4-(3-Fluoro-2-methyl-phenyl)-piperidin-1-yl]-[6-(4-methyl-piperazin-1-yl)-pyridin-2-yl]-methanone;

[4-(4-Fluoro-2-methyl-phenyl)-piperidin-1-yl]-[6-(4-methyl-piperazin-1-yl)-pyridin-2-yl]-methanone;

[4-(5-Fluoro-2-methyl-phenyl)-piperidin-1-yl]-[6-(4-methyl-piperazin-1-yl)-pyridin-2-yl]-methanone;

[4-(2-Fluoro-6-methyl-phenyl)-piperidin-1-yl]-[6-(4-methyl-piperazin-1-yl)-pyridin-2-yl]-methanone;

[4-(2-Fluoro-phenyl)-piperidin-1-yl]-[6-(4-methyl-piperazin-1-yl)-pyridin-2-yl]-methanone;

[4-(2,3-Difluoro-phenyl)-piperidin-1-yl]-[6-(4-methyl-piperazin-1-yl)-pyridin-2-yl]-methanone;

[4-(2,4-Difluoro-phenyl)-piperidin-1-yl]-[6-(4-methyl-piperazin-1-yl)-pyridin-2-yl]-methanone;

[4-(2,5-Difluoro-phenyl)-piperidin-1-yl]-[6-(4-methyl-piperazin-1-yl)-pyridin-2-yl]-methanone;

[4-(2,6-Difluoro-phenyl)-piperidin-1-yl]-[6-(4-methyl-piperazin-1-yl)-pyridin-2-yl]-methanone;

[4-Fluoro-4-(2-fluoro-phenyl)-piperidin-1-yl]-[6-(4-methyl-piperazin-1-yl)-pyridin-2-yl]-methanone;

(4-Fluoro-4-o-tolyl-piperidin-1-yl)-[6-(4-methyl-piperazin-1-yl)-pyridin-2-yl]-methanone;

6-(1-Methyl-pyrrolidin-3-yl)-pyridin-2-yl]-(4-o-tolyloxy-piperidin-1-yl)-methanone;

[4-(2-Fluoro-phenoxy)-piperidin-1-yl]-(6-pyrrolidin-3-yl-pyridin-2-yl)-methanone;

[4-(2-Chloro-phenoxy)-piperidin-1-yl]-[6-(1-methyl-pyrrolidin-3-yl)-pyridin-2-yl]-methanone; or

[6-(1-Methyl-pyrrolidin-3-yl)-pyridin-2-yl]-[4-(2-trifluoromethyl-phenoxy)-piperidin-1-yl]-methanone; or

pharmaceutically acceptable salts thereof.

Specific pyrrolidine embodiments of the invention also include:

[4-(4-Fluoro-2-methyl-phenoxy)-piperidin-1-yl]-[6-(1-methyl-pyrrolidin-3-yl)-pyridin-2-yl]-methanone;

[4-(2,5-Difluoro-phenoxy)-piperidin-1-yl]-[6-(1-methyl-pyrrolidin-3-yl)-pyridin-2-yl]-methanone;

[4-(2,4-Difluoro-phenyl)-piperidin-1-yl]-[6-(1-methyl-pyrrolidin-3-yl)-pyridin-2-yl]-methanone;

[4-(4-Fluoro-2-methyl-phenyl)-piperidin-1-yl]-[6-(1-methyl-pyrrolidin-3-yl)-pyridin-2-yl]-methanone;

[4-(5-Fluoro-2-methyl-phenyl)-piperidin-1-yl]-[6-(1-methyl-pyrrolidin-3-yl)-pyridin-2-yl]-methanone;

[4-(2-Fluoro-6-methyl-phenyl)-piperidin-1-yl]-[6-(1-methyl-pyrrolidin-3-yl)-pyridin-2-yl]-methanone; or

[4-(2-Fluoro-6-methyl-phenyl)-piperidin-1-yl]-[6-(3-fluoro-1-methyl-pyrrolidin-3-yl)-pyridin-2-yl]-methanone; or

pharmaceutically acceptable salts thereof.

For each of the aforesaid embodiments, it is understood that such embodiments are defined so as to include pharmaceutically acceptable salts as well as hydrates, solvates, isomers, crystalline and non-crystalline forms, isomorphs, polymorphs and metabolites thereof.

Generally, compounds of the present invention are 5-HT ligands. In particular, they can selectively bind to the 5-HT₆ receptor (e.g. receptor-specific agonists or antagonists). Thus, they are useful for treating diseases wherein modulation of 5-HT activity, specifically 5-HT₆ activity, is desired. Therefore, the compounds of this invention are useful for the treatment of diseases or disorders of the central nervous system. More specifically, for the treatment of psychosis, paraphrenia, psychotic depression, mania, schizophrenia, schizophreniform disorders, anxiety, migraine headache, drug addiction, convulsive disorders, personality disorders, post-traumatic stress syndrome, alcoholism, panic attacks, obsessive-compulsive disorders, eating disorders (e.g., binge eating disorder, anorexia, and bulimia), weight loss or control disorders (e.g., reduction in calorie or food intake, and/or appetite suppression), obesity or controlling weight gain (including reducing or maintaining weight) and sleep disorders. The compounds of this invention are also useful to treat psychotic, affective, vegetative, and psychomotor symptoms of schizophrenia and the extrapyramidal motor side effects of other antipsychotic drugs. This last action will allow higher doses of antipsychotics to be used and thus greater antipsychotic efficacy to be obtained as a result of a reduction in side effects. The compounds of this invention are also useful in the modulation of eating behavior and thus are useful in treating excess weight and associated morbidity and mortality.

The present invention further provides a method for treating diseases or disorders of the central nervous system comprising administering a therapeutically effective amount of a compound of Formula I, or a pharmaceutically acceptable salt thereof to the mammal. The term treating includes prophylactic treatment. In particular, compounds of Formula I are useful in treating depression, schizophrenia, schizophreniform disorder, and schizoaffective disorder. In some embodiments compounds of Formula I may have activity against other diseases or disorders including, but are not limited to, the following: obesity, delusional disorder, a stress related disease (e.g. general anxiety disorder), panic disorder, a phobia, obsessive compulsive disorder, post-traumatic-stress syndrome, immune system depression, a stress induced problem with the urinary, gastrointestinal or cardiovascular system (e.g., stress incontinence), neurodegenerative disorders, autism, chemotherapy-induced vomiting, hypertension, migraine headaches, cluster headaches, sexual dysfunction in a mammal (e.g. a human), addictive disorder and withdrawal syndrome, an adjustment disorder, an age-associated learning and mental disorder, anorexia nervosa, apathy, an attention-deficit disorder due to general medical conditions, attention-deficit hyperactivity disorder, behavioral disturbance (including agitation in conditions associated with diminished cognition (e.g., dementia, mental retardation or delirium)), bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, movement disorder, (e.g., Huntington's disease or Tardive Dyskinesia), oppositional defiant disorder, peripheral neuropathy, post-traumatic stress disorder, premenstrual dysphoric disorder, a psychotic disorder (brief and long duration disorders, psychotic disorder due to medical condition, psychotic disorder NOS), mood disorder (major depressive or bipolar disorder with psychotic features) seasonal affective disorder, a sleep disorder, a specific developmental disorder, agitation disorder, selective serotonin reuptake inhibition (SSRI) “poop out” syndrome or a Tic disorder (e.g., Tourette's syndrome).

The present invention further provides a method for treating anxiety, depression or stress related disorders comprising administering a therapeutically effective amount of a compound of formula I, or a pharmaceutically acceptable salt thereof to the mammal.

The present invention further provides the use of a compound of formula I or a pharmaceutically acceptable salt thereof to prepare a medicament for treating or preventing diseases or disorders of the central nervous system.

In another aspect, the invention provides a pharmaceutical composition comprising a therapeutically effective amount of a compound of formula I, or a pharmaceutically acceptable salt thereof. The composition may also include a pharmaceutically acceptable carrier.

The present invention further provides a method for treating a disease or condition in a mammal wherein a 5-HT receptor is implicated and modulation of a 5-HT function is desired comprising administering to the mammal a therapeutically effective amount of a compound of formula I, or a pharmaceutically acceptable salt thereof.

The term “therapeutically effective amount” as used herein refers to that amount of the compound being administered which will relieve to some extent one or more of the symptoms of the disorder being treated: In reference to the treatment of cognitive deficit, a therapeutically effective amount refers to that amount which has the effect of (1) reducing cognitive deficits in disease states such as schizophrenia and Alzheimer's mania where cognitive deficits are present, (2) inhibiting (that is, slowing to some extent, preferably stopping) cognitive decline in such disease states, (3) relieving to some extent (or, preferably, eliminating) one or more symptoms associated with cognitive decline in such disease states and/or (4) enhancing learning and memory in such disease states.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

The present invention also envisions the use of compounds of Formula I in combination with one or more additional CNS active agents which are described below. When a combination therapy is used, the one or more additional CNS active agents may be administered sequentially or simultaneously with the compound of the invention. In one embodiment, the additional CNS active agent is administered to a mammal (e.g., a human) prior to administration of the compound of the invention. In another embodiment, the additional CNS active agent is administered to the mammal after administration of the compound of the invention. In another embodiment, the additional CNS active agent is administered to the mammal (e.g., a human) simultaneously with the administration of the compound of the invention.

The invention also relates to a combination pharmaceutical composition, which comprises an amount of a compound of Formula I, as defined above (including pharmaceutically acceptable salts thereof), in combination with one or more (preferably one to three) CNS active agents selected from the group consisting of donepezil, clozapine, antischizophrenic aryl piperazole, quintapine, ziprasidone, wherein the amounts of the active agent and the combination CNS active agents when taken as a whole is therapeutically effective for treating schizophrenia.

As used herein, the term “combination therapy” refers to the administration of a compound of Formula I together with an at least one additional pharmaceutical or medicinal agent (e.g., an anti-schizophrenia agent), either sequentially or simultaneously.

The compounds of the invention may also be used in conjunction with other pharmaceutical agents for the treatment of the diseases/conditions described herein. Therefore, methods of treatment that include administering compounds of the present invention in combination with other pharmaceutical agents are also provided. Suitable pharmaceutical agents that may be used in combination with the compounds of the present invention include anti-obesity agents such as apolipoprotein-B secretion/microsomal triglyceride transfer protein (apo-B/MTP) inhibitors, 11β-hydroxy steroid dehydrogenase-1 (11β-HSD type 1) inhibitors, PYY₃₋₃₆ and analogs thereof, MCR4 agonists, cholecystokinin-A (CCK-A) agonists, monoamine reuptake inhibitors (such as sibutramine), sympathomimetic agents, β₃ adrenergic receptor agonists, dopamine agonists (such as bromocriptine), melanocyte-stimulating hormone receptor analogs, cannabinoid 1 receptor antagonists (e.g., rimonabant), melanin concentrating hormone antagonists, leptins (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y receptor antagonists (e.g., NPY Y5 receptor antagonists, such as the spiro compounds described in U.S. Pat. Nos. 6,566,367; 6,649,624; 6,638,942; 6,605,720; 6,495,559; 6,462,053; 6,388,077; 6,335,345; and 6,326,375; US Publication Nos. 2002/0151456 and 2003/036652; and PCT Publication Nos. WO 03/010175. WO 03/082190 and WO 02/048152), thyromimetic agents, dehydroepiandrosterone or an analog thereof, glucocorticoid receptor agonists or antagonists, orexin receptor antagonists, urocortin binding protein antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or inverse agonists, and neuromedin U receptor agonists. Other anti-obesity agents, including the preferred agents set forth hereinbelow, are well known, or will be readily apparent in light of the instant disclosure, to one of ordinary skill in the art.

Preferred are anti-obesity agents selected from the group consisting of orlistat, sibutramine, bromocriptine, ephedrine, leptin, rimonabant, pseudoephedrine, PYY₃₋₃₆ or an analog thereof, and 2-oxo-N-(5-phenylpyrazinyl)spiro-[isobenzofuran-1(3H),4′-piperidine]-1′-carboxamide.

Other suitable pharmaceutical agents that may be administered in combination with the compounds of the present invention include agents designed to treat tobacco abuse (e.g., nicotine receptor partial agonists, particularly Champix™, bupropion hypochloride (also known under the tradename Zyban™) and nicotine replacement therapies), ADD/ADHD treatment agents (e.g., Ritalin™, Strattera™, Concerta™ and Adderall™), and agents to treat alcoholism, such as opioid antagonists (e.g., naltrexone (also known under the tradename ReVia™) and nalmefene), disulfiram (also known under the tradename Antabuse™), and acamprosate (also known under the tradename Campral™)). In addition, agents for reducing alcohol withdrawal symptoms may also be co-administered, such as benzodiazepines, beta-blockers, clonidine, carbamazepine, pregabalin, and gabapentin (Neurontin™). Treatment for alcoholism is preferably administered in combination with behavioral therapy including such components as motivational enhancement therapy, cognitive behavioral therapy, and referral to self-help groups, including Alcohol Anonymous (AA). In addition to Zyban, other useful nicotine receptor partial agonists are described in U.S. Pat. Nos. 6,235,734; 6,410,550; and 6,462,035; all of which are incorporated herein by reference.

Other pharmaceutical agents that may be used in combination include antidepressants (e.g., fluoxetine hydrochloride (Prozac™)); and neuroprotective agents (e.g., memantine).

In another embodiment, compounds of the present invention are used in combination with cognitive improvement agents such as donepezil hydrochloride (Aricept™) and other acetylcholinesterase inhibitors; cannabinoid receptor 1 (CB1) antagonists; and alpha 7 nicotinic acetylcholine receptor agonists. Representative alpha 7 agonist compounds are listed in U.S. Pat. Nos. 6,911,543; 6,809,094; and 6,881,734, all of which are incorporated herein by reference.

According to a yet further aspect, the present invention additionally provides a method for the treatment and/or prevention of male sexual dysfunction via treatment with a combination of a compound of the present invention and at least one additional pharmaceutical agent. Preferred additional pharmaceutical agents used in treating male sexual dysfunction (e.g., male erectile dysfunction) include: (1) one or more dopaminergic agents (e.g. D2, D3 or D4 agonists and apomorphine); (2) one or more of an NPY (neuropeptide Y) (preferably an NPY-1 and/or NPY-5 inhibitor); (3) one or more of a melanocortin receptor agonist or modulator or melanocortin enhancer; (4) one or more of an NEP inhibitor; (5) one or more of a PDE inhibitor (preferably, a cGMP PDE-5 inhibitor); and (6) one or more of a bombesin receptor antagonist or modulator.

According to another aspect of the present invention, there is provided use of a compound of the present invention and one or more additional active agents for the treatment of female sexual dysfunction (FSD). Preferably, the one or more additional active agents is/are selected from the group consisting of: estrogen receptor modulators (e.g., estrogen agonists and/or estrogen antagonists); testosterone replacement agents and/or testosterone (Tostrelle) and/or dihydrotestosterone and/or dehydroepiandrosterone (DHEA) and/or a testosterone implant; estrogen, estrogen and medroxyprogesterone or medroxyprogesterone acetate (MPA) (as a combination), or a combination of estrogen and a methyl testosterone hormone replacement therapy agent; one or more dopaminergic agents; one or more NPY (neuropeptide Y) inhibitors; one or more melanocortin receptor modulators or melanocortin enhancers; one or more NEP (neutral endopeptidase) inhibitors; one or more PDE (phosphodiesterase) inhibitors; and one or more bombesin receptor modulators.

In another aspect, the compounds of the invention can be used in combination with other agents for the treatment of lower urinary tract dysfunction. Such other agents include: muscarinic acetylcholine receptor antagonists such as tolterodine; alpha adrenergic receptor antagonists, in particular an alpha1 adrenergic receptor antagonist or an alpha2 adrenergic receptor antagonist; alpha adrenergic receptor agonists or partial agonists, in particular an alpha1 adrenergic receptor agonist or partial agonist, or an alpha2 adrenergic receptor agonist or partial agonist; serotonin and noradrenalin reuptake inhibitor (SNRI); noradrenalin reuptake inhibitor (NRI) such as reboxetine, either in its racemic or (S,S)-enantiomeric form; vanilloid receptor (VR) antagonists, such as capsaicin; alpha2delta ligand, such as gabapentin or pregabalin; beta3 adrenergic receptor agonists; 5HT1a receptor antagonists or 5HT1a receptor inverse agonists; prostanoid receptor antagonists, e.g. EP1 receptor antagonist.

Suitable antipsychotic drugs useful for combination may be, for example, Chlorpromazine, Fluphenazine, Haloperidol, Loxapine, Mesoridazine, Molindone, Perphenazine, Pimozide, Thioridazine, Thiothixene, or Trifluoperazine. These drugs all have an affinity for the dopamine 2 receptor. The antipsychotic drug may also be, for example, Asenapine, Ziprasidone, Olanzapine, Clozapine, Risperidone, Sertindole, Quetiapine, Aripiprazole or Amisulpride.

### DETAILED DESCRIPTION

Compounds of the formula I may be prepared according to the following reaction schemes and discussion. Unless otherwise indicated, Y, Z, L, R¹ through R¹¹, n, m, p, q, t and structural formula I in the reaction schemes and discussion that follow are as defined above.

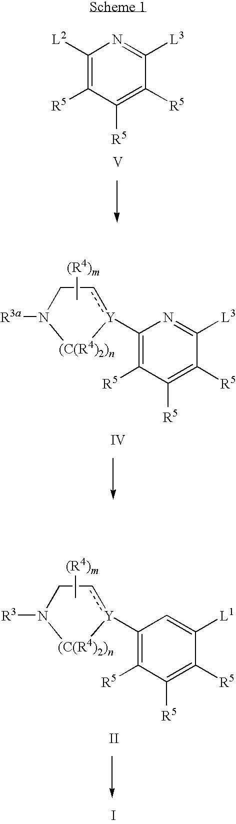

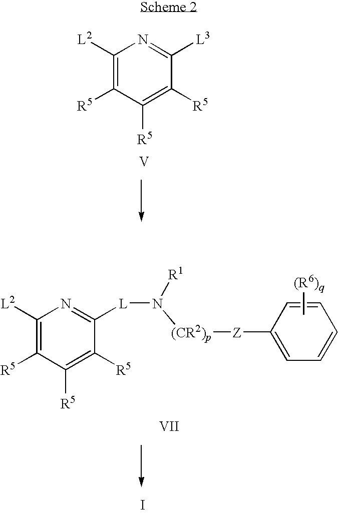

Referring to Scheme 1, compounds of the Formula I can be prepared from compounds of the Formula II by reacting said compound of the Formula II, wherein L¹ is an activated carbonyl or sulfonyl group, with a compound of the formula

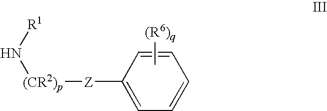

in the presence of a base and optionally containing an activating agent. Suitable bases include amines such as diisopropylethylamine, lutidine, pyrrolidine, triethylamine and pyridine, preferably diisopropylethylamine. Suitable activated carbonyl or sulfonyl groups include carboxylic acids, anhydrides, acid halides (preferably chlorides), pentafluorides, aryl esters, and suflonyl halides (preferably chlorides). Suitable activating agents include triazole uronium salts mixed with suitable additives. Suitable triazole uronium salts include 2-(7-aza-1H-benzotriazole-1-yl)-1,1,3,3-tetramethyl uronium hexafluorophosphate (HATU), 1-benzotriazol-1-yloxy-bis(pyrrolidino)uronium hexafluorophosphate (BBC), 5-(1H-benzotriazol-1-yloxy)-3,4-dihydro-1-methyl 2H-pyrrolium hexachloroanitimonate (BDMP), benzotriazol-1-yloxy-N,N-dimethylmethaniminium hexachloroantimonate (BOMI), O-(7-azabenzotriazol-1-yl)-1,1,3,3-bis(tetramethylene)uronium hexafluorophosphate (HAPyU), O-(benzotriazol-1-yl)-1,1,3,3-tetramethyluronium hexafluorophosphate (HBTU), and O-(7-azabenzotriazol-1-yl)-1,1,3,3-bis(pentamethylene)uronium tetrafluoroborate (TAPipU). Suitable additives include hydroxytriazole or pyridines, such as 1-hydroxy-7-azabenzotriazole (HOAT), 1-hydroxybenzotriazole (HOBT), and 3-hydroxy-3,4-dihydro-4-oxo-1,2,3-benzotriazine (HODHBT). Alternatively, the carbonyl group activating agent includes carbodiimides such as N,N-1,3-dicyclohexylcarbodiimide (DCC) and 1-ethyl-3-(3′-dimethylaminopropyl)carbodiimide hydrochloride (EDCI) in the presence of a catalyst such as N,N-dimethylaminopyridine (DMAP). Alternatively the carbonyl group activating agent can be a phosphonate such as diethyl cyanophosphonate in the presence of an amine base. Suitable solvents include polar solvents such as dimethyl sulfoxide (DMSO), dimethyl formamide (DMF), tetrahydrofuran (THF), dimethylacetamide (DMA), methylene chloride, and acetonitrile. Suitable reaction temperatures can range from about 0° C. to about 150° C., preferably from about 10° C. to about 25° C. (i.e. room temperature). The reaction is complete within about 0.5 hours to about 48 hours, preferably about 16 hours.

Compounds of the Formula II, wherein Y is >C(R⁷)— and the dashed line (- - -) is absent; or Y is carbon and the dashed line (- - -) is a double bond; or Y is >N— and the dashed line (- - -) is absent; L¹ is a carboxylic acid group (—(C═O)—OH), may be prepared from compounds of the Formula IV, wherein Y is >C(R⁷)— and the dashed line (- - -) is absent; or Y is carbon when the dashed line (- - -) is a double bond; or Y is >N— and the dashed line (- - -) is absent; and L³ is a nitrile group (—CN) or a protected acid group (—(C═O)—OP), by treatment with a hydrolyzing agent. Suitable hydrolyzing agents include acids such as hydrochloric acid or sulfuric acid in the presence of water or bases such as lithium, sodium or potassium hydroxide in the presence of water. “P” is a protecting group such as described in T. W. Greene and P. Wuts, Protecting Groups in Organic Synthesis, John Wiley & Sons, 2nd Edition, New York, 1991. Preferably the protecting group is an ester such as a methyl, ethyl, n-butyl or t-butyl ester. Alternatively, when the protected acid group is a benzyl ester, a hydrogenolysis reagent can be used to affect the transformation, preferably hydrogen over palladium/carbon. Suitable reaction temperatures can range from about 25° C. to about 100° C., preferably about 100° C. (i.e. boiling temperature). The reaction is complete within about 0.5 hours to about 24 hours, preferably about 16 hours.

Compounds of the Formula II, wherein L¹ is a sulfonic acid can be prepared from compounds of Formula IV, wherein L³ is a sulfonate ester by treatment with a base, such as sodium hydroxide is a solvent such as an alcohol or alcohol water mixture such as ethanol, or methanol. Suitable reaction temperatures can range from about 0° C. to about 100° C., preferably about 60° C. The reaction is complete within about 4 hours to about 24 hours, preferably about 12 hours.

Compounds of the Formula IV, wherein Y is >N— and the dashed line (- - -) is absent, can be prepared by coupling compounds of the formula V, wherein L² is a halide such as chloro, bromo or iodo, and L³ is a protected carboxylic or sulfonic acid equivalent such as nitrile, or —(C═O)—O—P or —SO₂—O—P, with a compound of the formula

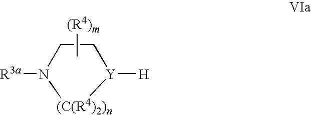

wherein Y is nitrogen and R^{3a} is a nitrogen protecting group or a group described above as R³, in the presence of a base. Suitable bases include K₂CO₃, K₃PO₄, Cs₂CO₃, LiN(TMS)₂ or an alkoxide base such as sodium methoxide, sodium ethoxide, potassium t-butoxide, preferably sodium or potassium carbonate. Suitable solvents include DMF, DMSO, toluene, dioxane or an ethereal solvent, preferably DMF or DMSO. The aforesaid reaction may be run at a temperature of about 40° C. to 110° C. for about 1 to 48 hours.

Alternatively, said coupling can be facilitated by treatment of the halide under so called Buchwald conditions, such as by treatment with a palladium catalyst in the presence of a phosphine ligand such as 2-dicyclohexylphosphinobiphenyl. Such conditions are reviewed in Angew. Chem. Int. Ed. Engl. 1998, 37, 2046-2067 and are well known to those of ordinary skill in the art. Preferred Buchwald conditions use palladium acetate (Pd(OAc)₂) or palladium tetra-triphenylphosphine (Pd(PPh₃)₄) as the source of the palladium.

Alternatively, compounds of the Formula II, wherein Y is >C(R⁷)—, the dashed line (- - -) is absent and R⁷ is hydrogen, and L¹ is a protected acid group (—(C═O)—OP), or a carboxylic acid, and the dashed line (- - -) is absent may be prepared from compounds of the Formula IV, wherein Y is carbon and the dashed line (- - -) is a double bond and L¹ is a protected carboxylic or sulfonic acid group (e.g., —(C═O)—OP), or a carboxylic acid, or sulfonic acid by treatment with a reducing agent. Reduction may be effected with hydrogen gas (H₂), using catalysts such as palladium on carbon (Pd/C), palladium on barium sulfate (Pd/BaSO4), platinum on carbon (Pt/C), or tris(triphenylphosphine) rhodium chloride (Wilkinson's catalyst), in an appropriate solvent such as methanol, ethanol, THF, dioxane or ethyl acetate, at a pressure from about 1 to about 5 atmospheres and a temperature from about 10° C. to about 60° C., as described in “Catalytic Hydrogenation in Organic Synthesis”, Paul Rylander, Academic Press Inc., San Diego, 31-63 (1979). The following conditions are preferred: Pd on carbon, methanol at 25° C. and 50 psi of hydrogen gas pressure. This method also provides for introduction of hydrogen isotopes (i.e., deuterium, tritium) by replacing ¹H₂ with ²H₂ or ³H₂ in the above procedure. Suitable reducing agents also include sodium borohydride (NaBH₄), sodium cyanoborohydride (NaCNBH₃), lithium aluminum hydride (LiAlH₄) and borane in THF (BH₃.THF) in solvents such as methanol, ethanol, THF, diethyl ether and dioxane.

Compounds of the Formula IV, wherein Y is carbon and the dashed line (- - -) is a double bond, can be prepared from compounds of the Formula V by reaction with a compound of the formula

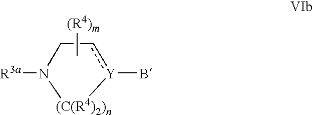

wherein Y is carbon, the dashed line (- - -) is a double bond, B′ is a boronate ester and R^{3a} is a nitrogen protecting group or a group described above as R³, in the presence of a catalyst. Suitable catalysts include palladium catalysts such as [1,1′-bis(diphenylphosphino)ferrocene] palladium (II), tris(dibenzylidene acetone)dipalladium(O) (Pd₂(dba)₃), di(dibenzylidene acetone) palladium(O) (Pd(dba)2), palladium acetate (Pd(OAc)₂, and a suitable ligand, such as a triaryl phosphine ligand, tri(t-butyl)phosphine, 1,1-bis(diphenylphosphanyl)ferrocene (DPPF), 2,2′-bis(diphenylphosphanyl)-1,1′-binaphthyl (BINAP), or PHANEPHOS, preferably tri(ortho-tolyl)phosphine. Suitable bases include K₂CO₃, K₃PO₄, Cs₂CO₃, LiN(TMS)₂ or an alkoxide base such as sodium methoxide, sodium ethoxide, potassium t-butoxide, preferably sodium tert-butoxide. Suitable solvents include toluene or an ethereal solvent, preferably dioxane. The aforesaid reaction may be run at a temperature of about 40° C. to 110° C. for about 1 to 48 hours. Such conditions are reviewed in Akira Suzuki, “Recent advances in the cross-coupling reactions of organoboron dervatives with organic electrophiles, 1995-1998,” Journal of Organometallic Chemistry (1999), 576(1-2), 147-168; and Paul R. Eastwood, “A versatile synthesis of 4-aryltetrahydropyridines via palladium mediated Suzuki cross-coupling with cyclic vinyl boronates,” Tetrahedron Letters (2000), 41(19), 3705-3708, and are well known to those of ordinary skill in the art. Suitable solvents include THF, toluene or ethereal solvents. The aforesaid reaction may be run at a temperature of about 25° C. to 110° C. for about 1 to 4 hours, preferably 2 hours. Nickel catalysts, such as Ni(cod) (nickel 1,5-cyclooctadiene), are also well known, see for example Vijaya Gracias and Rajesh Iyengar, “Recent advances in nickel-catalyzed Suzuki cross-coupling reactions,” Chemtracts (2005), 18(6), 339-348.

Alternatively, compounds of Formula IV, wherein Y is carbon, and the dashed line (- - -) is a double bond, a so called “reverse Suzuki” coupling, can be prepared by reaction of said compound of formula V, wherein L² is borate or boronic acid, with a compound of formula

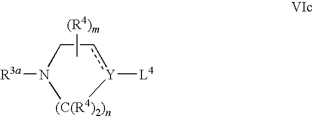

wherein Y is carbon, the dashed line (- - -) is a double bond, L⁴ is halo (preferably Br or I) or triflate, in the presence of a catalyst, a base and a dehydrating agent. Suitable borates include (HO)₂B—, 9-BBN, and alkylboranes. Suitable catalysts include copper or palladium (such as palladium acetate (Pd(OAc)₂), palladium triphenylphosphine or Pd(dppf)Cl₂), preferably copper (II) acetate. Suitable dehydrating agents include 4 angstrom molecular sieves. Suitable bases include tertiary amine bases, such as triethylamine or pyridine, Na₂CO₃, sodium ethoxide, and K₃PO₄. Suitable solvents include methylene chloride, dimethyl sulfoxide (DMSO) or tetrahydrofuran (THF). The aforesaid reaction is typically performed under an atmosphere of oxygen gas at a temperature of about 10° C. to 50° C., preferably about 23° C. for about 6 to 72 hours. Palladium-catalyzed boronic acid couplings are described in Miyaura, N., Yanagi, T., Suzuki, A. Syn. Comm. 1981, 11, 7, p. 513.

Alternatively, compounds of Formula IV, wherein Y is >C(R⁷)—, R⁷ is hydroxyl, and the dashed line (- - -) is absent, can be prepared from compounds of Formula V, wherein L² is halide (preferably bromide) by reaction with a compound of Formula

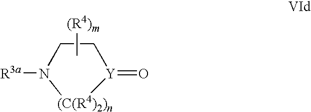

wherein Y is carbon, by a so called metal-halogen exchange reaction. One skilled in the art will appreciate that the halide is preconditioned with a strong base to facilitate the metal exchange followed by addition of the carbonyl compound. Suitable solvents include ethers such as diethylether, dioxane, glyme or tetrahydrofuran (THF), preferably THF. The aforesaid reaction is typically performed under an inert atmosphere at a temperature of about −80° C. to 60° C., preferably about −80° C. for the exchange reaction followed by allowing the reaction to equilibrate at 0° C. during coupling or even heating the reaction if necessary for from about 1 to 12 hours.

Compounds of Formula IV, wherein R⁷ is halo (preferably fluoro), can be prepared from compounds of Formula IV, wherein R⁷ is hydroxyl, with diethylaminosulfurtrifluoride (DAST), SELECTFLUOR® or Deoxo-Fluor™ (Air Products) in an inert solvent such as methylene chloride. The aforesaid reaction is typically performed under an inert atmosphere at a temperature of about −80° C. to 80° C., preferably about 22° C. (room temperature) for from about 1 to 24 hours.

Compounds of formula IV, wherein Y is >C(R⁷)—, the,dashed line (- - -) is absent, and R⁷ is (C₁-C₆)alkyl, can be prepared from compounds of Formula IV, wherein Y is carbon and the dashed line (- - -) is a double bond, by treatment with a strong base (such as butyl lithium) a carbon electrophile (such as dimethyl sulfate) in an ethereal solvent (such as diethylether, dioxane, glyme or tetrahydrofuran (THF), preferably THF) at a temperature of about O° C. to form an intermediate R⁷ alkyl enamine that can be converted to the desired amine product of Formula IV by reduction in the presence of a mild reducing agent (such as sodium borohydride) in an alcohol solvent (such as methanol or ethanol) and preferably at O° C.

Compounds of Formula IV, wherein Y is carbon and the dashed line (- - -) is a double bond, can be prepared from compounds of Formula IV, wherein the dashed line (- - -) is absent, Y is >C(R⁷)— and R⁷ is hydroxyl, by reaction with a dehydrating reagent such as triflic anhydride and methanesulfonyl chloride, in the presence of a base, such as potassium carbonate or sodium hydride. Suitable solvents include ethers such as diethylether, dioxane, glyme or tetrahydrofuran (THF), preferably THF. The aforesaid reaction is typically performed at a temperature of about 0° C. to 100° C. for from about 1 to 24 hours.

Compounds of the Formula V are commercially available or can be made by methods well known to those skilled in the art.

Referring to Scheme 2, compounds of the Formula I may be prepared from compounds of the formula VII, wherein L² is as defined in Scheme 1, by reaction with a compound of the Formula VI, VIa, VIb, VIc or VId according to methods analogous to those described above for the conversion of compounds of Formula V to compounds of Formula IV in Scheme 1.

Compounds of formula VII may be prepared from compounds of the formula V by reaction with a compound of the formula III according to methods analogous to those described above for the conversion of compounds of Formula II to compounds of Formula I in Scheme 1.

Compounds of Formulae I, II, III, IV and VII that have chiral centers may exist as stereoisomers, such as racemates, enantiomers, or diastereomers. Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of Formula I (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomeric conglomerates may be separated by conventional techniques known to those skilled in the art. See, e.g. “Stereochemistry of Organic Compounds” by E. L. Eliel (Wiley, New York, 1994), the disclosure of which is incorporated herein by reference in its entirety.

Where a compound of Formula I contains an alkenyl or alkenylene group, geometric cis/trans (or Z/E) isomers are possible. Cis/trans isomers may be separated by conventional techniques well known to those skilled in the art, for example, chromatography and fractional crystallization. Salts of the present invention can be prepared according to methods known to those of skill in the art.

The compounds of Formula I that are basic in nature are capable of forming a wide variety of salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

Those compounds of Formula I that are acidic in nature are capable of forming base salts with various pharmacologically acceptable cations. These salts are all prepared by conventional techniques, for example, treatment of the free acid with an inorganic or organic base, such as an amine (primary, secondary or tertiary), an alkali metal hydroxide or alkaline earth metal hydroxide, or the like. These salts can also be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

For a review on suitable salts, see Handbook of Pharmaceutical Salts: Properties, Selection, and Use by Stahl and Wermuth (Wiley-VCH, 2002).

Polymorphs can be prepared according to techniques well-known to those skilled in the art such as those described in Grant, David J. W., “Polymorphism in Pharmaceutical Solids,” Drugs and the Pharmaceutical Sciences, 95, Chapter 1, pp. 1-33 and Chapter 5, pp. 183-219. Publisher: Marcel Dekker, Inc. (1999).

Isotopically-labeled compounds of Formula I can generally be prepared by conventional techniques known to those skilled in the art or by processes analogous to those described herein, using an appropriate isotopically-labeled reagent in place of the non-labeled reagent otherwise employed.

Prodrugs in accordance with the invention can, for example, be produced by replacing appropriate functionalities present in the compounds of Formula I with certain moieties known to those skilled in the art as ‘pro-moieties’ as described, for example, in Design of Prodrugs by H. Bundgaard (Elsevier, 1985).

The ability of a compound of the invention to act as a 5-HT receptor agonist or antagonist can also be determined using in vitro and in vivo assays that are known in the art. All of the Example compounds provided herein are 5-HT ligands, with the ability to displace >50% of a radiolabeled test ligand from one or more 5-HT receptor subtypes at a concentration of 1 μM. The procedures used for testing such displacement are well known and illustrated below.

5-HT₆ Receptor Binding Assay

Growth of Cells and Membrane Preparation

Hela cells containing the cloned human 5-HT₆ receptor were acquired from Dr. David R. Sibley's laboratory in National Institute of Health (see Sibley, D. R., J. Neurochemistry, 66, 47-56, 1996). Cells were grown in high glucose Dulbecco's modified Eagle's medium, supplemented with L-glutamine, 0.5% sodium pyruvate, 0.3% penicillin-streptomycin, 0.025% G-418 and 5% Gibco fetal bovine serum and then were harvested, when confluent, in cold phosphate buffered saline.

Harvested intact cells were washed once in cold phosphate-buffered saline. The cells were pelleted and resuspended in 100 ml of cold 50 mM Tris, 5 mM EDTA and 5 mM EGTA, pH 7.4. Homogenization was with a Vir Tishear generator, 4 cycles for 30 seconds each at setting 50. The homogenized cells were centrifuged at 700 RPM (1000×g) for 10 minutes and the supernatant was removed. The pellet was resuspended in 100 ml of the above buffer and rehomogenized for 2 cycles. The rehomogenized cells were then centrifuged at 700 RPM (1000×g) for 10 minutes and the supernatant was removed. The combined supernatant (200 ml) was centrifuged at 23,000 RPM (80,000×g) for 1 hour in a Beckman Rotor (42.1 Ti). The membrane pellet was resuspended in 50-8-ml of assay buffer containing HEPES 20 mM, MgCl₂ 10 mM, NaCl 150 mM, EDTA 1 mM, pH 7.4 and stored frozen in aliqouts at −70° C.

5-HT₆ Receptor Binding Assay

The radioligand binding assay used [³H]-lysergic acid diethylamide (LSD). The assay was carried out in Wallac 96-well sample plates by the addition of 11 μl of the test sample at the appropriate dilution (the assay employed 11 serial concentrations of samples run in duplicate), 11 μl of radioligand, and 178 μl of a washed mixture of WGA-coated SPA beads and membranes in binding buffer. The plates were shaken for about 5 minutes and then incubated at room temperature for 1 hour. The plates were then loaded into counting cassettes and counted in a Wallac MicroBeta Trilux scintillation counter.

Binding Constant (Ki) Determination

Binding Constant Determination may be obtained by performing serial dilutions, e.g., eleven dilutions, of test compounds into assay plates using the PE/Cetus Pro/Pette pipetter. These dilutions are followed by radioligand and the bead-membrane mixture prepared as described above. After obtaining the specifically bound cpm, the data are fit to a one-site binding model using GraphPad Prism ver. 2.0. Estimated IC₅₀ values are converted to Ki values using the Cheng-Prusoff equation (Cheng, Y. C. et al., Biochem. Pharmacol., 22, 3099-108, 1973).

Obesity and Related Disorders

Spontaneous Food Intake

The following screen is used to evaluate the efficacy of test compounds for inhibiting spontaneous food intake in Sprague-Dawley rats.

Male Sprague-Dawley rats may be obtained from Charles River Laboratories, Inc. (Wilmington, Mass.). The rats are individually housed and fed powdered chow. They are maintained on a 12 hour light/dark cycle and received food and water ad libitum. The animals are acclimated to the vivarium for a period of one week before testing is conducted. Rats are transferred to individual test cages 30 hours before the study. The rats are administered test compound or vehicle alone (no compound) 15-30 minutes prior to the onset of the dark cycle. The test compounds are dosed at ranges between 0.1 and 100 mg/kg depending upon the compound. The standard vehicle is 0.5% (w/v) methylcellulose or 30% β-cyclodextrin in water and the standard route of administration is oral. However, different vehicles and routes of administration are used to accommodate various compounds when required.

Food intake is monitored using an automated Columbus Instruments system (Columbus, Ohio). Individual rat food intake is recorded continuously at 10-minute intervals, starting at the time of dosing, for a period of at least 12 hours. Compound efficacy is determined by comparing the food intake pattern of compound-treated rats to vehicle.

Compounds of the present invention can conveniently be administered in a pharmaceutical composition containing the compound in combination with a suitable excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15th Ed., 1975). The compounds and compositions of the present invention can be administered parenterally (for example, by intravenous, intraperitoneal or intramuscular injection), topically, orally, or rectally.

For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices including, but not limited to, those relying on osmotic pressures to obtain a desired release profile (e.g., the OROS drug delivery devices as designed and developed by Alza Corporation).

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. Sterilization of the powders may also be accomplished through irradiation and aseptic crystallization methods. The sterilization method selected is the choice of the skilled artisan.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. To this extent, the present invention further contemplates the use of the pharmaceutically active materials in personal care compositions such as lotions, cleansers, powders, cosmetics and the like.

The compound is conveniently administered in unit dosage form; for example, containing about 0.05 mg to about 500 mg, conveniently about 0.1 mg to about 250 mg, most conveniently, about 1 mg to about 150 mg of active ingredient per unit dosage form. The desired dose may conveniently be presented in a single dose or as divided doses administered at appropriate intervals, for example, as two, three, four or more sub-doses per day. The sub dose itself may be further divided, e.g., into a number of discrete loosely spaced administrations.

The compositions can conveniently be administered orally, sublingually, transdermally, or parenterally at dose levels of about 0.01 to about 150 mg/kg, preferably about 0.1 to about 50 mg/kg, and more preferably about 0.1 to about 30 mg/kg of mammal body weight.

For parenteral administration the compounds are presented in aqueous solution in a concentration of from about 0.1 to about 10%, more preferably about 0.1 to about 7%. The solution may contain other ingredients, such as emulsifiers, antioxidants or buffers.

The exact regimen for administration of the compounds and compositions disclosed herein will necessarily be dependent upon the needs of the individual subject being treated, the type of treatment and, of course, the judgment of the attending practitioner.

Generally, compounds of the invention are 5-HT ligands. The ability of a compound of the invention to bind or act at a 5-HT receptor, or to bind or act selectively at a specific 5-HT receptor subtype can be determined using in vitro and in vivo assays that are known in the art. As used herein, the term “bind selectively” means a compound binds at least 2 times, preferably at least 10 times, and more preferably at least 50 times more readily to a given 5-HT subtype than to one or more other subtypes. Preferred compounds of the invention bind selectively to one or more 5-HT receptor subtypes. Most preferred compounds of the invention bind selectively to the 5-HT6 receptor subtype.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The compounds and their preparations of the present invention will be better understood in connection with the following examples, which are intended as an illustration of and not a limitation upon the scope of the invention.

In the following Examples and Preparations, “BOC”, “Boc” or “boc” means N-tert-butoxycarbonyl, “DCM” (CH₂Cl₂) means methylene chloride, “DIPEA” or “DIEA” means diisopropyl ethyl amine, “DMA” means N,N-dimethylacetamide, “DMF” means N-N-dimethyl formamide, “DMSO” means dimethylsulfoxide, “DPPP” means 1,3-bis(diphenylphosphino)propane, “HOAc” means acetic acid, “IPA” means isopropyl alcohol. “MTBE” means methyl t-butyl ether, “NMP” means 1-methyl 2-pyrrolidinone, “TEA” means triethyl amine, “TFA” means trifluoroacetic acid, “DCM” means dichloromethane, “EtOAc” means ethyl acetate, “MgSO₄” means magnesium sulphate, “NaSO₄” means sodium sulphate, “MeOH” means methanol, “EtOH” means ethanol, “H₂O” means water, “HCl” means hydrochloric acid, “POCl₃” means phosphorus oxychloride, “DMSO” means dimethyl sulfoxide, “K₂CO₃” means potassium carbonate, “N” means Normal, “M” means molar, “mL” means millilitre, “mmol” means millimoles, “μmol” means micromoles, “eq.” means equivalent, “° C.” means degrees Celsius, “Pa” means pascals.

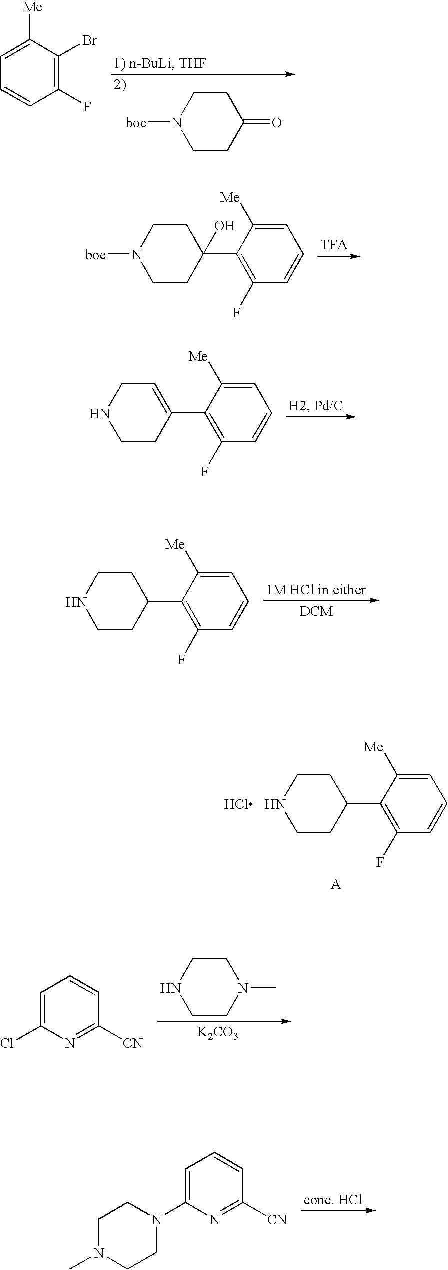

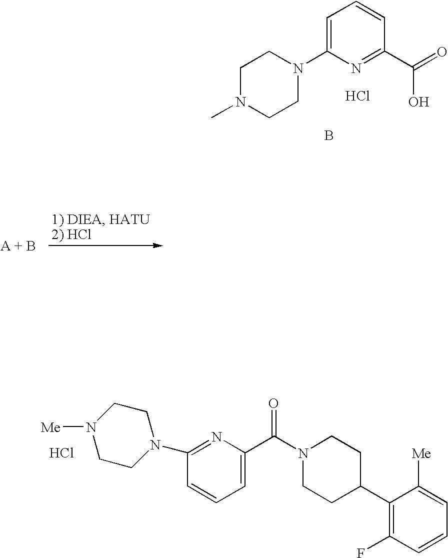

### EXAMPLE 1

### 6-(4-Methyl-piperazin-1-yl)-pyridine-2-carbonitrile

To a solution of 6-chloro-pyridine-2-carbonitrile (425 g, 3.08 mol, 98%, Matrix) in DMF (4500 ml) was added N-methylpiperazine (924 g, 9.24 mol, 98%, Aldrich) and K₂CO₃ (1700 g, 12.32 mol, 99%, anhydrous, Aldrich). The resulting reaction mixture was stirred at 100° C. overnight. The solvent was concentrated and the residue partitioned between EtOAc and water. The organic layer was separated, dried, and concentrated to give 620 g (99%) of 6-(4-methyl-piperazin-1-yl )-pyridine-2-carbonitrile.

### 6-(4-Methyl-piperazin-1-yl)-pyridine-2-carboxylic acid

To 6-(4-methyl-piperazin-1-yl)-pyridine-2-carbonitrile (318 g, 1.57 mol) was added concentrated hydrochloric acid (2500 mL, 36%, Caledon) and the mixture refluxed overnight, cooled, and concentrated. To the residue was added MeOH (200 mL), followed by Et₂O (2500 mL) with stirring resulting in a precipitate. The solids were filtered, washed with small amount of MeOH, and dried to furnish 500 g (96%) of 6-(4-methyl-piperazin-1-yl)-pyridine-2-carboxylic acid hydrochloride as a white solid.

### 4-(2-Fluoro-6-methyl-phenyl)-4-hydroxy-piperidine-1-carboxylic acid tert-butyl ester

To a solution of 2-bromo-1-fluoro-3-methylbenzene (3.0 g, 15.9 mmol) in 70 mL of THF cooled to −78° C. was added n-BuLi (14.9 mL, 23.8 mmol, 1.6 M soln in hexane) dropwise. The mixture was stirred at −78° C. for 30 min at which time 1-Boc-4-piperidone (3.16 g, 15.9 mmol) in 15 mL of THF was added dropwise. The mixture was warmed to ambient temperature, quenched with saturated ammonium chloride, extracted with ethyl acetate, washed with water, brine, dried over sodium sulfate and concentrated. The residue was purified by column chromatography (20% EtOAc/Hexane) to afford 4-(2-fluoro-6-methyl-phenyl)-4-hydroxy-piperidine-1-carboxylic acid tert-butyl ester as a white solid (3.0 g, 61%). ¹H NMR 400 MHz (CDCl₃) δ 7.10 (m, 1H), 6.95 (d, 1H), 6.85 (m, 1H), 4.10-3.90 (m, 2H), 3.30-3.20 (m, 2H), 2.60 (s, 3H), 2.42-2.30 (m, 2H), 1.90-1.80 (m, 2H), 1.50 (s, 9H).

### 4-2-Fluoro-6-methyl-phenyl)-3,6-dihydro-2H-pyridine-1-carboxylic acid tert-butyl ester

A solution of 4-(2-fluoro-6-methyl-phenyl)-4-hydroxy-piperidine-1-carboxylic acid tert-butyl ester (3.0 g, 9.7 mmol) in 20 mL of trifluoroacetic acid was heated to 100° C. for 1.5 h. The mixture was cooled and concentrated. The residue was dissolved in ethyl acetate, neutralized with sat. NaHCO₃ (aq), washed with water, brine, dried (Na₂SO₄), and concentrated to afford 4-(2-fluoro-6-methyl-phenyl)-3,6-dihydro-2H-pyridine-1-carboxylic acid tert-butyl ester as an off-white solid (1.37 g, 74%). ¹H NMR 400 MHz (CDCl₃) δ 7.15 (m, 1H), 6.98 (d, 1H), 6.85 (t, 1H), 5.60 (s, 1H), 3.70 (m, 2H), 3.30 (m, 2H), 2.45 (m, 2H), 2.25 (s, 3H).

### 4-(2-Fluoro-6-methyl-phenyl)-piperidine-1-carboxylic acid tert-butyl ester

To a solution of 4-(2-fluoro-6-methyl-phenyl)-3,6-dihydro-2H-pyridine-1-carboxylic acid tert-butyl ester (1.25 g, 6.54 mmol) in 60 mL of MeOH was added Pd—C (1.2 g, 10% Pd, wet). The reaction mixture was hydrogenated under 50 psi at room temperature for 40 h, filtered through Celite and concentrated to afford 4-(2-fluoro-6-methyl-phenyl)-piperidine-1-carboxylic acid tert-butyl ester as a gum (0.75 g, 59.5%). ¹H NMR 400 MHz (CDCl₃) δ 7.10 (m, 1H), 6.90 (d, 1H), 6.85 (m, 1H), 3.50 (m, 2H), 3.08-2.92 (m, 3H), 2.50-2.40 (m, 2H), 2.38 (s, 3H), 1.80 (d, 2H).

### 4-(2-Fluoro-6-methyl-phenyl)-piperidine hydrochloride

A solution of 4-(2-fluoro-6-methyl-phenyl)-piperidine-1-carboxylic acid tert-butyl ester (0.75 g, 3.9 mmol) in 5 mL of dichloromethane was cooled to 0° C. and 1 M HCl in ether (3.9 mL, 3.9 mmol) was added dropwise. The mixture was stirred at ambient temperature for 30 min, concentrated, and washed with ether to afford 4-(2-fluoro-6-methyl-phenyl)-piperidine hydrochloride as a white solid (0.65 g, 73%). ¹H NMR 400 MHz (DMSO-d₆) δ 7.18 (m, 1H), 7.02 (d, 1H), 6.98 (m, 1H), 3.35 (m, 2H), 3.15-3.00 (m, 3H), 2.40 (s, 3H), 2.20 (d, 1H), 1.78 (d, 2H).

### [4-(2-Fluoro-6-methyl-phenyl)-piperidin-1-yl]-[6-(4-methyl-piperazin-1-yl)-pyridin-2-yl]-methanone

To a mixture of 6-(4-methyl-piperazin-1-yl)-pyridine-2-carboxylic acid hydrochloride (0.25 g, 0.70 mmol), 4-(2-fluoro-6-methyl-phenyl)-piperidine hydrochloride (0.16 g, 0.70 mmol) in 5 mL of DMF was added diisopropylethylamine (0.85 g, 4.9 mmol), and 2-(7-aza-1H-benzotriazole-1-yl)-1,1,3,3-tetramethyl uronium hexafluorophosphate (HATU) (0.32 g, 0.84 mmol). The mixture was stirred at ambient temperature overnight, extracted with EtOAc, washed with sat. NaHCO₃ (aq), water, brine, dried (Na₂SO₄), and concentrated. The residue was purified by column chromatography (5% MeOH/CH₂Cl₂) to afford [4-(2-fluoro-6-methyl-phenyl)-piperidin-1-yl]-[6-(4-methyl-piperazin-1-yl)-pyridin-2-yl]-methanone as a white foam (0.18 g, 65%). MS m/z 397 (M+1). ¹H NMR 400 MHz (CDCl₃) δ 7.58 (t, 1H), 7.05 (m, 1H), 6.96-6.84 (m, 3H), 6.66 (d, 1H), 4.90 (d, 1H), 4.12 (d, 1H), 3.60 (t, 4H), 3.06 (t, 2H), 2.80 (t, 1 H), 2.50 (t, 4H), 2.38 (s, 3H), 2.34 (s, 3H), 2.30-2.20 (m, 2H), 1.78 (d, 1H), 1.72 (d, 2H).

### [4-(2-Fluoro-6-methyl-phenyl)-piperidin-1-yl]-[6-(4-methyl-piperazin-1-yl)-pyridin-2-yl]-methanone hydrochloride

A solution of [4-(2-fluoro-6-methyl-phenyl)-piperidin-1-yl]-[6-(4-methyl-piperazin-1-yl)-pyridin-2-yl]-methanone (0.18 g, 0.45 mmol) in 2 mL of dichloromethane was cooled to 0° C. at which time 1.36 mL of 1M HCl in ether was added dropwise. The mixture was stirred at ambient temperature for 1 h, concentrated, and washed with ether to provide [4-(2-fluoro-6-methyl-phenyl)-piperidin-1-yl]-[6-(4-methyl-piperazin-1-yl)-pyridin-2-yl]-methanone hydrochloride as a white solid (0.155 g, 73%): MS m/z 397 (M+1), HPLC 96.9%, mp 163-164.7° C. ¹H NMR 400 MHz (CD₃OD) δ 7.75 (t, 1H), 7.10 (m, 1H), 7.05 (d, 1H), 7.00-6.90 (m, 2H), 6.88-6.80 (m, 1H), 4.80 (d, 1H), 4.55 (d, 2H), 3.90 (d, 1H), 3.60 (d, 2H), 3.30-3.10 (m, 6H), 3.00 (s, 3H), 2.95 (m, 1H), 2.40 (s, 3H), 2.25-2.10 (m, 2H), 1.80 (d, 1H), 1.65 (d, 1H).

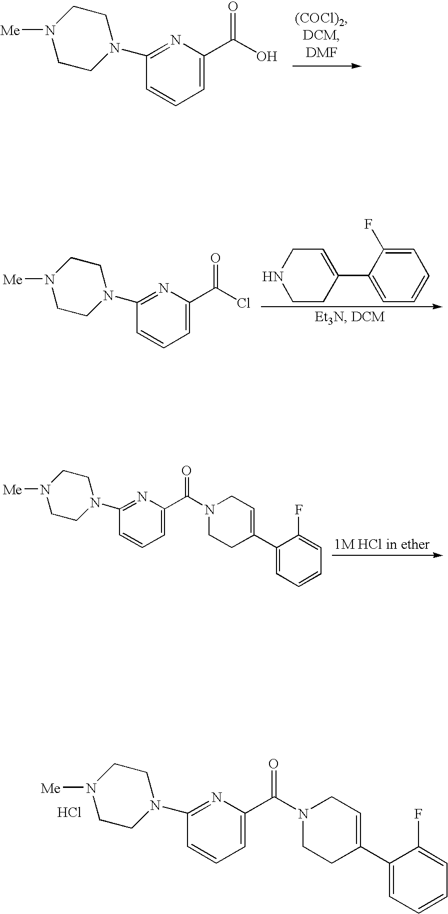

### EXAMPLE 2

[4-(2-Fluoro-phenyl)-3,6-dihydro-2H-pyridin-1-yl]-[6-(4-methyl-piperazin-1-yl)-pyridin-2-yl]-methanone.

To a mixture of 6-(4-methyl-piperazin-1-yl)-pyridine-2carboxylic acid hydrochloride (0.9 g, 2.51 mmol) in 40 mL of dichloromethane was added oxalyl chloride (0.64 g, 5.03 mmol) followed by two drops of DMF. The mixture was stirred at ambient temperature for 1 h and concentrated. To the crude 6-(4-methyl-piperazin-1-yl)-pyridine-2-carbonyl chloride (0.41 g, 1.0 mmol) in 20 mL of dichloromethane was added 4-(2-fluoro-phenyl)-1,2,3,6-tetrahydro-pyridine (0.18 g, 1.0 mmol), and triethylamine (0.60 g, 6.0 mmol). The mixture was stirred at ambient temperature overnight, extracted with dichloromethane, washed with water, brine, dried (Na₂SO₄), and concentrated. The residue was purified by column chromatography (5% MeOH/CH₂Cl₂) to afford [4-(2-fluoro-phenyl)-3,6-dihydro-2H-pyridin-1-yl]-[6-(4-methyl-piperazin-1-yl)-pyridin-2-yl]-methanone as a white foam (0.15 g, 39%). ¹H NMR 400 MHz CDCl₃) δ 7.55 (t, 1H), 7.30-7.20 (m, 1H), 7.15-6.98 (m, 3H), 6.70 (d, 1H), 6.00-5.85 (m, 1H), 4.40-4.30 (m, 2H), 4.00-3.75 (m, 2H), 3.62-3.55 (m, 4H), 2.62 (m, 2H), 2.52-2.42 (m, 4H), 2.35 (s, 3H).

### [4-(2-Fluoro-phenyl)-3,6-dihydro-2H-pyridin-1-yl]-[6-(4-methyl-piperazin-1-yl)-pyridin-2-yl]-methanone hydrochloride

To a solution of [4-(2-fluoro-phenyl)-3,6-dihydro-2H-pyridin-1-yl]-[6-(4-methyl-piperazin-1-yl)-pyridin-2-yl]-methanone (0.14 g, 0.37 mmol) in 5 mL of dichloromethane at 0° C. was added 1.0 mL of 1M HCl in ether dropwise. The mixture was stirred at ambient temperature for 1 h, concentrated, and washed with ether to afford [4-(2-fluoro-phenyl)-3,6-dihydro-2H-pyridin-1-yl]-[6-(4-methyl-piperazin-1-yl)-pyridin-2-yl]-methanone as a yellow solid (0.14 g, 81%): MS m/z 381 (M+1), HPLC 93%, which was repurified by preparative HPLC to 97.6%, mp 176.7-177.2° C. ¹H NMR 400 MHz (CD₃OD) δ 7.80 (t, 1H), 7.35-7.25 (m, 2H), 7.15 (t, 1H), 7.10-7.00 (m, 3H), 6.05-5.90 (m, 1H), 4.55 (d, 2H), 4.35-4.20 (m, 2H), 3.95 (t, 1H), 3.70 (t, 1H), 3.60 (d, 2H), 3.25 (d, 2H), 3.22 (d, 2H), 2.95 (s, 3H), 2.68-2.60 (m, 2H).

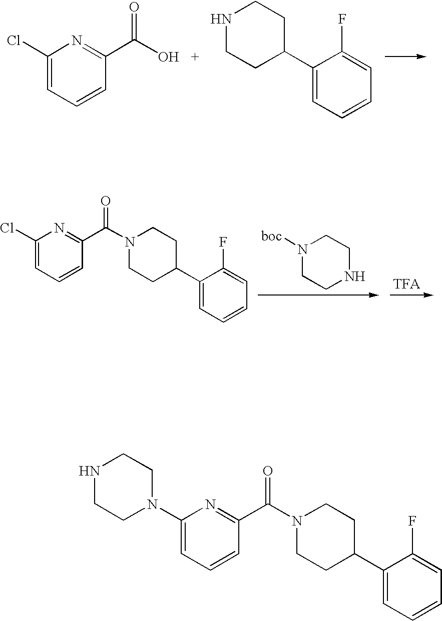

### EXAMPLE 3

### (6-Chloro-pyridin-2-yl)-[4-(2-fluoro-phenyl)-piperidin-1-yl]-methanone

A mixture of 6-chloro-pyridine-2-carboxylic acid (2.00 g, 12.7 mmol), 4-(2-fluoro-phenyl)-piperidine (2.50 g, 14 mmol)), diisopropylethylamine (4.4 mL, 25 mmol), and HATU (5.8 g, 15 mmol) in 40 mL DMF was stirred at room temperature for 18 h, then diluted with water and EtOAc. The phases were separated and the organic phase washed with brine, dried (Na₂SO₄), concentrated, and adsorbed on silica gel. Chromatography on silica gel eluting with a gradient of EtOAc/heptane gave (6-chloro-pyridin-2-yl)-[4-(2-fluoro-phenyl)-piperidin-1-yl]-methanone as an off-white solid (3.85 g, 95%). ¹H NMR (400 MHz, CHLOROFORM-d) δ ppm 1.64-2.01 (m, 4 H) 2.83-2.93 (m, 1 H) 3.10-3.22 (m, 2 H) 3.98-4.08 (m, 1 H) 4.81-4.88 (m, 1 H) 6.97-7.03 (m, 1 H) 7.04-7.11 (m, 1 H) 7.13-7.24 (m, 2 H) 7.28-7.39 (m, 1 H) 7.49-7.58 (m, 1 H) 7.67-7.79 (m, 1 H). MS [M+H]=3.19.

### [4-(2-Fluoro-phenyl)-piperidin-1-yl]-(6-piperazin-1-yl-pyridin-2-yl)-methanone

A mixture of (6-chloro-pyridin-2-yl)-[4-(2-fluoro-phenyl)-piperidin-1-yl]-methanone (0.40 g, 1.25 mmol)), 1-Boc-piperazine (0.35 g, 1.9 mmol), sodium t-butoxide (0.17 g, 1.76 mmol), 2-dicyclohexylphosphinobiphenyl (44 mg, 0.125 mmol), and palladium acetate (14 mg, 0.062 mmol) in 10 mL toluene was degassed with nitrogen and then heated to 110° C. for 18 h. The mixture was cooled and diluted with EtOAc, washed with water and brine, dried (Na₂SO₄), concentrated, and adsorbed on silica gel. Chromatography on silica gel eluting with 10% 2N NH3 in methanol/CH₂Cl₂ furnished 4-{6-[4-(2-fluoro-phenyl)-piperidin-1-carbonyl]-pyridin-2-yl}-piperazin-1-carboxylic acid tert-butyl ester as a yellow foam (0.41 g): LCMS, >97%. The carbamate was dissolved in 5 mL DCM and treated with 2 mL TFA. The solution was stirred at ambient temperature for 4 h, then added to 50 mL sat. NaHCO₃ (aq) and extracted with CH₂Cl₂. The organic phase was separated, dried (Na₂SO₄), concentrated, and adsorbed on silica gel. Chromatography eluting with 10% 2N NH3 in methanol/CH₂Cl₂ provided [4-(2-fluoro-phenyl)-piperidin-1-yl]-(6-piperazin-1-yl-pyridin-2-yl)-methanone as a pale yellow glass (0.27 g, 58%). ¹H NMR (400 MHz, CD3OD) δ ppm 1.50-2.00 (m, 5 H), 2.20-2.40 (m, 1 H) 2.60-3.30 (m, 6 H) 3.49 (m, 4 H) 4.12 (m, 1 H) 4.86 (m, 1 H) 6.62 (m, 1 H) 6.80-7.30 (m, 5 H) 7.50 (m, 1 H). MS [M+H]=369.

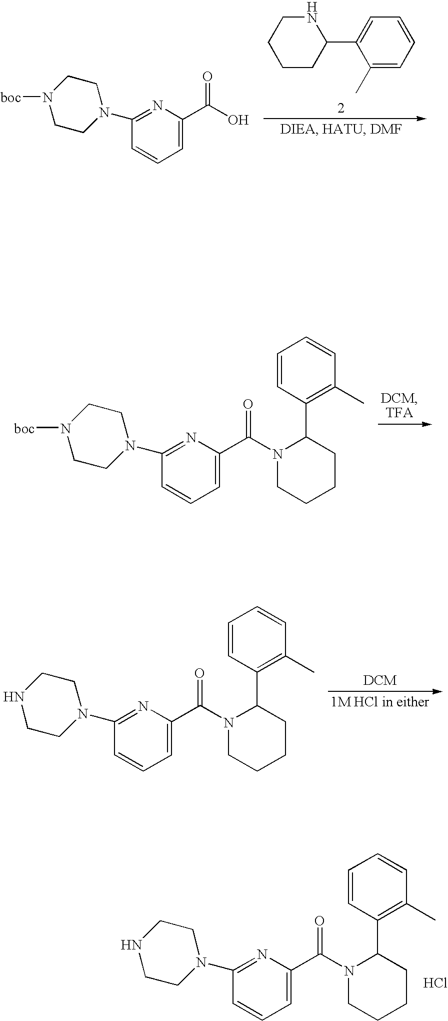

### EXAMPLE 4

### 4-[6-(2-o-Tolyl-piperidine-1-carbonyl)-pyridin-2-yl]-piperazine-1-carboxylic acid tert-butyl ester

A mixture of 4-(6-carboxy-pyridin-2-yl)-piperazine-1-carboxylic acid tert-butyl ester (0.307 g, 1.0 mmol), 2-o-tolyl-piperidine (0.23 g, 1.1 mmol) in 5 mL of DMF and diisopropylethylamine (0.28 g, 2.2 mmol), 2-(7-aza-1H-benzotriazole-1-yl)-1,1,3,3-tetramethyl uronium hexafluorophosphate (HATU, 0.42 g, 1.1 mmol) was stirred at ambient temperature overnight. The mixture was extracted with EtOAc, washed with saturated NaHCO₃, water, brine, dried (Na2SO4), and concentrated. The residue was purified by column chromatography (20-30% EtOH/hexane) to afford 4-[6-(2-o-tolyl-piperidine-1-carbonyl)-pyridin-2-yl]-piperazine-1-carboxylic acid tert-butyl ester as a white foam (0.34 g, 73%). ¹H NMR 400 MHz (CDCl₃) δ 7.60-7.50 (m, 1H), 7.48-7.40 (m, 2H), 7.20-6.88 (m, 3H), 6.65-6.50 (m, 1H), 5.94 (br s, ½H), 5.20 (br s, ½H), 4.80 (d, ½H), 3.78 (d, ½H), 3.60-3.45 (m, 6H), 4.40-3.35 (m, 2H), 3.20-3.10 (m, 2H), 2.40 (s, 3H), 2.20-2.10 (m, 1H), 2.00-1.85 (m, 2H), 1.80-1.65 (m, 2H), 1.50 (s, 9H).

### (6-Piperazin-1-yl-pyridin-2-yl)-(2-o-tolyl-piperidin-1-yl)-methanone

A solution of 4-[6-(2-o-tolyl-piperidine-1-carbonyl)-pyridin-2-yl]-piperazine-1-carboxylic acid tert-butyl ester (0.34 g, 0.81 mmol) in 6 mL of dichloromethane and 2 mL of trifluoroacetic acid was stirred at room temperature overnight, saturated NaHCO₃ was added, extracted with dichloromethane, washed with water, brine, dried over sodium sulfate and concentrated to afford (6-piperazin-1-yl-pyridin-2-yl)-(2-o-tolyl-piperidin-1-yl)-methanone as a white solid (0.29 g, quantitative). ¹H NMR 400 MHz (CDCl₃) δ 7.60-7.40 (m, 3H), 7.20-6.80 (m, 3H), 6.70-6.50 (m, 1H), 5.95 (br s, ½H), 5.25 (br s, ½H), 4.80 (d, ½H), 3.80 (d, ½H), 3.60-3.45 (m, 2H), 3.25-3.10 (m, 2H), 3.00-2.78 (m, 4H), 2.40 (s, 3H), 2.20-1.85 (m, 4H), 1.70-1.58 (m, 4H).

### (6-piperazin-1-yl-pyridin-2-yl)-(2-o-tolyl-piperidin-1-yl)-methanone hydrochloride

To a solution of (6-piperazin-1-yl-pyridin-2-yl)-(2-o-tolyl-piperidin-1-yl)-methanone (0.29 g, 0.79 mmol), in 5 mL of dichloromethane and 1 mL of methanol, 1 mL of 1M HCl in ether was added dropwise. The reaction mixture was stirred at room temperature for 30 min, the reaction mixture was concentrated, washed with ether to furnish (6-piperazin-1-yl-pyridin-2-yl)-(2-o-tolyl-piperidin-1-yl)-methanone hydrochloride as a light yellow solid (0.32 g, quantitative). MS m/z 365 (M+1), HPLC 95.02%, mp=105.6-106° C. ¹H NMR 400 MHz (CD₃OD) δ 7.75-7.50 (m, 1H), 7.40-7.30 (m, 1H), 7.20-7.05 (m, 3H), 6.95-6.70 (m, 1H), 5.70 (br s, ½H), 5.00 (br s, ½H), 3.80-3.60 (m, 2H), 3.40-3.25 (m, 2H), 3.20-2.90 (m, 6H), 2.40-2.20 (m, 2H), 1.90 (s, 3H), 1.80-1.40 (m, 4H), 1.10 (s, 1 H).

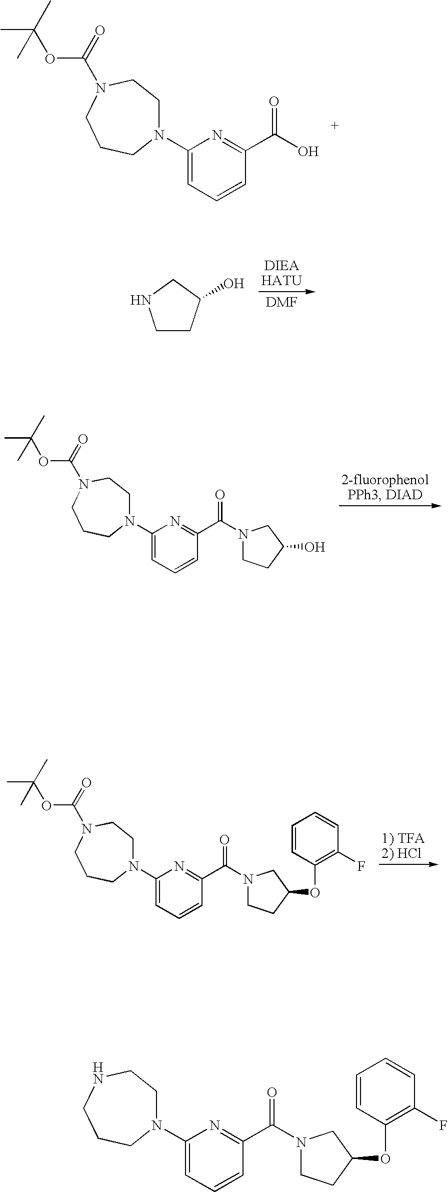

### EXAMPLE 5

### [6-((R)-3-Hydroxy-pyrrolidine-1-carbonyl)-pyridin-2-yl]-[1,4]diazepane-1-carboxylic acid tert-butyl ester

A mixture of 4-(6-carboxy-pyridin-2-yl)-[1,4]diazepane-1-carboxylic acid tert-butyl ester (0.80 g, 2.50 mmol), (R)-pyrrolidin-3-ol (0.24 g, 2.75 mmol), O-(7-azabenzotriazol-1-yl)-N,N,N′,N′-tetramethyluronium hexafluorophosthate (HATU, 1.14 g, 3.0 mmol), and N,N-diisopropylethylamine (0.48 g, 3.75 mmol) in DMF (10 mL) was stirred at room temperature overnight, then water (20 mL) was added. The mixture was extracted with EtOAc (50 mL×3), dried over Na₂SO₄, and concentrated. Silica gel chromatography (CH₂Cl₂:MeOH, 95:5) gave 4-[6-((R)-3-hydroxy-pyrrolidine-1-carbonyl)-pyridin-2-yl]-[1,4]diazepane-1-carboxylic tert-butyl ester (0.95 g, crude) as a white sticky mass that was used in the next step without further purification. MS m/z 391 [C₂₀H₃₀N₄O₄+1].

### 4-{6-[(S)-3-(2-Fluoro-phenoxy)-pyrrolidine-1-carbonyl]-pyridin-2-yl}-[1,4]diazepane-1-carboxylic acid tert-butyl ester

To a mixture of 4-[6-((R)-3-hydroxy-pyrrolidine-1-carbonyl)-pyridin-2-yl]-[1,4]diazepane-1-carboxylic acid tert-butyl ester (270 mg, 0.69 mmol), 2-fluorophenol (0.07 mL, 0.76 mmol) and triphenylphosphine (272 mg, 1.04 mmol) in THF (7.5 mL) was added at 0° C. diisopropyl azodicarboxylate (0.20 mL, 1.04 mmol). The reaction mixture was stirred overnight at 60° C., then concentrated in vacuo. Silica gel chromatography (hexane/ethyl acetate, 1:2) of the residue gave a mixture of 4-{6-[(S)-3-(2-fluoro-phenoxy)-pyrrolidine-1-carbonyl]-pyridin-2-yl}-[1,4]diazepane-1-carboxylic acid tert-butyl ester and triphenylphosphine oxide that was used in the next step without further purification. ¹H NMR (400 MHz, CDCl₃): δ 7.20-6.83 (m, 6H), 6.55 (dd, 1H), 4.98 (br. s, 1H), 4.20-3.15 (m, 12H), 2.39-1.82 (m, 4H), 1.43-1.41 (2s, 9H). MS m/z 485 [C₂₆H₃₃FN₄O₄+1].

### (6-[1,4]Diazepan-1-yl-pyridin-2-yl)-[(S)-3-(2-fluoro-phenoxy)-pyrrolidin-1-yl]-methanone

To a solution of 4-{6-[(S)-3-(2-fluoro-phenoxy)-pyrrolidine-1-carbonyl]-pyridin-2-yl}-[1,4]diazepane-1-carboxylic acid tert-butyl ester (crude 400 mg) in dichloromethane (6 mL) was added trifluoroacetic acid (2 mL). The reaction mixture was stirred overnight at rt, then cooled to 0° C. Water was added (10 mL) and the pH of the solution was adjusted to 8 by adding sodium bicarbonate in small portions. After extraction with dichloromethane (3×20 mL) the filtrate was dried over magnesium sulfate and concentrated in vacuo. Silica gel chromatography (dichloromethane/methanol/ammonium hydroxyde, 9:1:0.2) of the residue gave (6-[1,4]diazepan-1-yl-pyridin-2-yl)-[(S)-3-(2-fluoro-phenoxy)-pyrrolidin-1-yl]-methanone (100 mg, 38%) as a sticky yellow residue. ¹H NMR (400 MHz, CDCl₃): δ 7.61 (m, 1H), 7.27-7.09 (m, 3H), 6.97 (m, 2H), 6.75 (dd, 1H), 5.13 (br. s, 1H), 4.00-3.60 (m, 8H), 2.99-2.77 (m, 4H), 2.16 (m, 2H), 1.86-1.76 (m, 2H), 1NH not seen. HPLC (X5 Acidic): 92.42% MS m/z 385 [C₂₁H₂₅FN₄O₂+1].

### (6-[1,4]Diazepan-1-yl-pyridin-2-yl)-[(S)-3-(2-fluoro-phenoxy)-pyrrolidin-1-yl]-methanone hydrochloride

To a solution of 3 (75 mg, 0.20 mmol) in CH₂Cl₂ (2 mL) was added 1 mL of HCl (1M in ether) dropwise at rt. The reaction mixture was stirred at room temperature for 1 hour, then was concentrated in vacuo. The residue was triturated in ether and dried by decantation to give (6-[1,4]diazepan-1-yl-pyridin-2-yl)-[(S)-3-(2-fluoro-phenoxy)-pyrrolidin-1-yl]-methanone hydrochloride (72 mg, 85%) as a yellow foam, nip=75-77° C. ¹H NMR (400 MHz, DMSO-d₆): δ 9.02 (br. s, 2H), 7.64 (dd, 1H), 7.25-7.12 (m, 3H), 7.06-6.92 (m, 2H), 6.82 (dd, 1H), 5.12 (br. s, 1H), 4.10-3.57 (m, 8H), 3.25-3.06 (m, 4H), 2.21-1.97 (m, 4H). HPLC (X5 Acidic): 95.04%. MS m/z 385 [C₂₁H₂₅FN₄O₂+1].

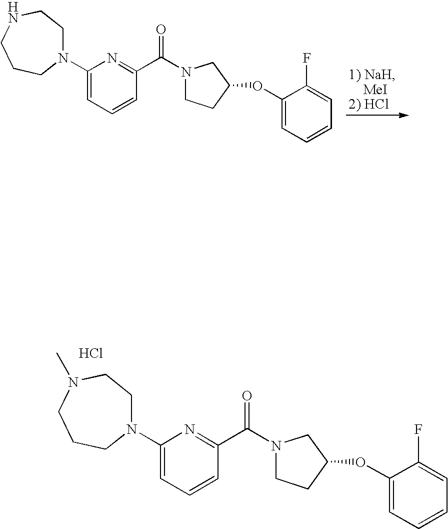

### Example 6

### [(R)-3-(2-Fluoro-phenoxy)-pyrrolidin-1-yl]-[6-(4-methyl-[1,4]diazepan-1-yl)-pyridin-2-yl]-methanone

Sodium hydride (65 mg, 1.63 mmol) was added at 0° C. to a solution of (6-[1,4]diazepan-1-yl-pyridin-2-yl )-[(R)-3-(2-fluoro-phenoxy)-pyrrolidin-1-yl]-methanone (627 mg, 1.63 mmol) in THF (50 mL). A 0.1M solution of iodomethane in THF (16.3 mL, 1.63 mmol) was then added at rt and the reaction mixture stirred for 45 minutes. Water was added (50 mL) and the aqueous layer was extracted with dichloromethane (3×100 mL). The combined organic extracts were dried over magnesium sulfate, filtered and concentrated in vacuo. Silica gel chromatography (ethyl acetate/methanol/triethylamine, 8:1.5:0.5) of the residue gave [(R)-3-(2-fluoro-phenoxy)-pyrrolidin-1-yl]-[6-(4-methyl-[1,4]diazepan-1-yl)-pyridin-2-yl]-methanone (101 mg, 16%) as a sticky residue. ¹H NMR (400 MHz, CDCl₃): δ 7.56-7.51 (m, 1H), 7.20-6.91 (m, 5H), 6.53-6.51 (dd, 1H), 4.99 (br. s, 1H), 4.20-3.79 (m, 6H), 3.67-3.57 (m, 2H), 2.77-2.58 (m, 4H), 2.43-2.39 (m, 3H), 2.38-1.99 (m, 4H). HPLC (X5 Acidic): 96.25%. MS m/z 399 [C₂₂H₂₇FN₄O₂+1].

### [(R)-3-(2-Fluoro-phenoxy)-pyrrolidin-1-yl]-[6-(4-methyl-[1,4]diazepan-1-yl)-pyridin-2-yl]-methanone hydrochloride

To a solution of [(R)-3-(2-fluoro-phenoxy)-pyrrolidin-1-yl]-[6-(4-methyl-[1,4]diazepan-1-yl)-pyridin-2-yl]-methanone (101 mg, 0.25 mmol) in CH₂Cl₂ (4 mL) was added 2 mL of HCl (1M in ether) dropwise at rt. The reaction mixture was stirred at room temperature for 1 hour, then was concentrated in vacuo. The residue was triturated in ether and dried by decantation to give [(R)-3-(2-fluoro-phenoxy)-pyrrolidin-1-yl]-[6-(4-methyl-[1,4]diazepan-1-yl)-pyridin-2-yl]-methanone hydrochloride (108 mg, 98%) as a yellow foam, mp=106-108° C. ¹H NMR (400 MHz, DMSO-d₆): δ 10.58 (br. s, 1H), 7.71-7.65 (dd, 1H), 7.28-6.94 (m, 5H), 6.85-6.79 (dd, 1H), 5.12 (br. s, 1H), 4.30-3.36 (m, 8H), 3.20-3.01 (m, 4H), 2.79-2.74 (d, 3H), 2.46-2.16 (m, 4H). HPLC (X5 Acidic): 96.94%. MS m/z 399 [C₂₂H₂₇FN₄O₂+1].

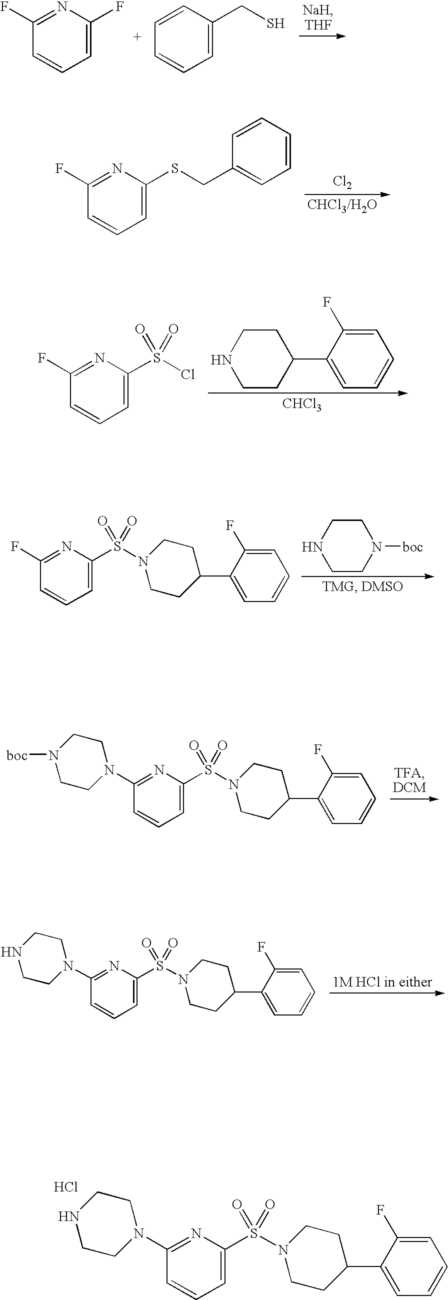

### EXAMPLE 7

### 2-Benzylsulfanyl-6-fluoro-pyridine

A solution of benzyl mercaptan (5.4 g, 39.7 mmol) in THF (25 mL) was added dropwise to a stirred suspension of sodium hydride (2.4 g, 59.5 mmol, 60% dispersion in oil) in THF (50 mL) under nitrogen atmosphere. The mixture was stirred for 30 min at which time a solution of 2,6-difluoropyridine (5.0 g, 43.4 mmol) in THF (25 mL) was added dropwise. The mixture was stirred at ambient temperature overnight and then carefully quenched with water, extracted with Et₂O, washed with water, brine, dried (Na₂SO₄), and concentrated. The residue was purified by column chromatography (5% EtOH/hexane) to afford 2-benzylsulfanyl-6-fluoro-pyridine as a clear liquid (9.2 g, 92%). ¹H NMR 400 MHz (CDCl₃) δ 7.58 (q, 1H), 7.60 (d, 2H), 7.30 (t, 1H), 7.25 (d, 2H), 7.02 (d, 1H), 6.60 (d, 1H), 4.40 (s, 2H).

### 6-Fluoro-pyridine-2-sulfonyl chloride

Chlorine gas was bubbled through a vigorously stirred mixture of 2-benzylsulfanyl-6-fluoro-pyridine (0.5 g, 1.04 mmol) in 25 mL of chloroform and 25 mL of water for 95 min. The mixture was diluted with sodium metabisulfite (aq), extracted with chloroform, washed with water, brine, dried (Na₂SO₄), and concentrated to afford 6-fluoro-pyridine-2-sulfonyl chloride as a clear oil (0.7 g, 78%). ¹H NMR 400 MHz (CDCl₃) δ 8.20 (q, 1H), 8.00 (d, 1H), 7.60 (d, 1H).

### 2-Fluoro-6-[4-(2-fluoro-phenyl)-piperidine-1-sulfonyl]pyridine

To a solution of 6-fluoro-pyridine-2-sulfonyl chloride (0.2 g, 1.02 mmol) in chloroform (10mL) at 0° C. was added 4-(2-fluoro-phenyl)-piperidine hydrochloride (0.16 g, 0.77 mmol) in 5 mL of chloroform dropwise. The mixture was stirred for 10 min, then quenched with water, extracted with chloroform, washed with water, brine, dried (Na₂SO₄), and concentrated. The residue was purified by column chromatography (100% CH₂Cl₂) to afford 2-fluoro-6-[4-(2-fluoro-phenyl)-piperidine-1-sulfonyl]pyridine as a white solid (0.1 g, 38%). ¹H NMR 400 MHz (CDCl₃) δ 8.02 (t, 1H), 7.85 (d, 1H), 7.20-7.10 (m, 3H), 7.08 (d, 1H), 7.00 (t, 1H), 4.05 (d, 2H), 3.00-2.90 (m, 3H), 1.90-1.80 (m, 4H). 4-{6-[4-(2-Fluoro-phenyl)-piperidine-1-sulfonyl]-pyridin-2-yl}-piperazine-1-carboxylic acid tert-butyl ester

A mixture of 2-fluoro-6-[4-(2-fluoro-phenyl)-piperidine-1-sulfonyl]pyridine (0.1 g, 0.295 mmol), 1-Boc-piperazine (0.071 g, 0.38 mmol), and 1,1,3,3-tetramethylguanidine (0.068 g, 0.59 mmol) in 2 mL DMSO was heated at 80° C. overnight. The mixture was cooled and extracted with EtOAc, washed with water, brine, dried (Na₂SO₄), and concentrated. The residue was purified by column chromatography (4% MeOH/CH₂Cl₂) to afford 4-{6-[4-(2-fluoro-phenyl)-piperidine-1-sulfonyl]-pyridin-2-yl}-piperazine-1-carboxylic acid tert-butyl ester as a white foam (0.11 g, 74%). ¹H NMR 400 MHz (CDCl₃) δ 7.62 (t, 1H), 7.28 (s, 1H), 7.20-7.18 (m, 2H), 7.15 (d, 1H), 7.00 (t, 1H), 6.80 (d, 1H), 4.10 (d, 2H), 3.62-3.52 (m, 8H), 2.90-2.80 ( m, 3H), 1.90-1.82 (m, 4H), 1.50 (s, 9H).

### 1-{6-[4-(2-Fluoro-phenyl)-piperidine-1-sulfonyl]-pyridin-2-yl}-piperazine

A solution of 4-{6-[4-(2-fluoro-phenyl)-piperidine-1-sulfonyl]-pyridin-2-yl}-piperazine-1-carboxylic acid tert-butyl ester (0.11 g, 0.21 mmol) in 5 mL of dichloromethane and 2 mL of trifluoroacetic acid was stirred at ambient temperature overnight. The mixture was partitioned between sat. NaHCO₃ (aq) and dichloromethane, the phases were separated, and the organic phase washed with water, brine, dried (Na₂SO₄), and concentrated. The residue was purified by preparative thin layer chromatography (7% MeOH/CH₂Cl₂) to afford 1-{6-[4-(2-fluoro-phenyl)-piperidine-1-sulfonyl]-pyridin-2-yl}-piperazine as a white solid (0.04 g, 45%). ¹H NMR 400 MHz (CDCl₃) δ 7.70 (t, 1H), 7.35 (d, 1H), 7.20-7.15 (m, 2H), 7.10 (t, 1H), 7.00 (t, 1H), 6.82 (d, 1H), 4.05 (d, 2H), 3.90-3.82 (m, 4H), 3.38-3.22 ( m, 4H), 2.98-2.82 (m, 1H), 2.80 (t, 2H), 1.90-1.80 (m, 4H).

### 1-{6-[4-(2-Fluoro-phenyl)-piperidine-1-sulfonyl]-pyridin-2-yl}-piperazine hydrochloride

To a solution of 1-{6-[4-(2-fluoro-phenyl)-piperidine-1-sulfonyl]-pyridin-2-yl}-piperazine (0.38 g, 0.099 mmol), in 3 mL of dichloromethane and 2 mL of methanol, 1 mL of 1 M HCl in ether was added dropwise. The reaction mixture was stirred at room temperature for 30 min, the reaction mixture was concentrated, washed with ether to furnish 1-{6-[4-(2-fluoro-phenyl)-piperidine-1-sulfonyl]-pyridin-2-yl}-piperazine hydrochloride as a white solid (0.045 g, quantitative): MS m/z 405 (M+1), HPLC 95.2%, mp 211-212.2° C. ¹H NMR 400 MHz (CD₃OD) δ 7.75 (t, 1H), 7.22 (d, 1H), 7.18-7.00 (m, 4H), 6.85 (t, 1H), 3.90 (d, 2H), 3.82-3.75 (m, 4H) 3.30-3.20 (m, 4H), 2.80-2.70 (m, 3H), 1.80-1.65 (m, 4H).

### 2-Fluoro-6-(4-o-tolyl-piperidine-1-sulfonyl)-pyridine

To a solution of 6-fluoro-pyridine-2-sulfonyl chloride (0.4 g, 2.05 mmol) in chloroform (10 mL) at 0° C. was added 4-o-tolyl-piperidine hydrochloride (0.3 g, 1.42) in 10 mL of chloroform dropwise. The mixture was stirred for 10 min, then quenched with water, extracted with chloroform, washed with water, brine, dried (Na₂SO₄), and concentrated. The residue was purified by column chromatography (100% CH₂Cl₂) to afford 2-fluoro-6-(4-o-tolyl-piperidine-1-sulfonyl)-pyridine as a white solid (0.18 g, 38%). ¹H NMR 400 MHz (CDCl₃) δ 8.05-8.00 (m, 1H), 7.90 (d, 1H), 7.20-7.10 (m, 5H), 4.10 (d, 2H), 3.00-2.90 (m, 2H), 2.82-2.75 (m, 1H), 2.35 (s, 3H), 1.90-1.80 (m, 4H).

### 1-Methyl-4-[6-(4-o-tolyl-piperidine-1-sulfonyl)-pyridin-2-yl]-piperazine

A mixture of 2-fluoro-6-(4-o-tolyl-piperidine-1-sulfonyl)-pyridine (0.18 g, 0.54 mmol), 1-methyl-piperazine (0.08 g, 0.81 mmol), and 1,1,3,3-tetramethylguanidine (0.124 g, 1.1 mmol) in 3 mL DMSO was heated to 80° C. overnight. The mixture was cooled and extracted with EtOAc, washed with water, brine, dried (Na₂SO₄), and concentrated. The residue was purified by column chromatography (3% MeOH/CH₂Cl₂) to afford 1-methyl-4-[6-(4-o-tolyl-piperidine-1-sulfonyl)-pyridin-2-yl]-piperazine as a white foam (0.2 g, 90%). ¹H NMR 400 MHz (CDCl₃) δ 7.62 (t, 1H), 7.28 (s, 1H), 7.20-7.10 (m, 4H), 6.80 (d, 1H), 4.08 (d, 2H), 3.68-3.60 (m, 4H), 2.88-2.70 ( m, 3H), 2.56-2.50 (m, 4H), 2.36 (s, 3H), 2.30 (s, 3H), 1.86-1.76 (m, 4H).

### 1-Methyl-4-[6-(4-o-tolyl-piperidine-1-sulfonyl)-pyridin-2-yl]-piperazine hydrochloride

To a solution of 1-methyl-4-[6-(4-o-tolyl-piperidine-1-sulfonyl)-pyridin-2-yl]-piperazine (0.2 g, 0.48 mmol) in 5 mL of dichloromethane was added 1 mL of 1M HCl in ether dropwise. The mixture was stirred at ambient temperature for 30 min, concentrated, and washed with ether to yield 1-methyl-4-[6-(4-o-tolyl-piperidine-1-sulfonyl)-pyridin-2-yl]-piperazine hydrochloride as a light yellow solid (0.2 g, 88%): MS m/z 415 (M+1), HPLC 95.6%, mp 203-204.5° C. ¹H NMR 400 MHz (DMSO-d₆) δ 7.88 (t, 1H), 7.25 (t, 2H), 7.18-7.04 (m, 4H), 4.45 (d, 2H), 3.85 (d, 2H), 3.60-3.45 (m, 2H) 3.40-3.35 (m, 2H), 3.20-3.00 (m, 2H), 2.85-2.75 (m, 6H), 2.25 (s, 3H), 1.75 (d, 2H), 1.70-1.60 (m, 2H).

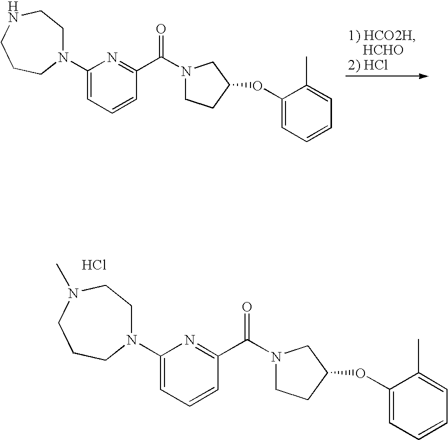

### EXAMPLE 8

### [6-(4-Methyl-[1,4]diazepan-1-yl)-pyridin-2-yl]-((R)-3-o-tolyloxy-pyrrolidin-1-yl)-methanone

To a solution of (6-[1,4]diazepan-1-yl-pyridin-2-yl)-((R)-3-o-tolyloxy-pyrrolidin-1-yl)-methanone (190 mg, 0.50 mmol) in formic acid (2 mL) was added formaldehyde (37% in water, 0.4 mL) at rt. The reaction mixture was heated at 100° C. for 15 minutes and it was then quenched with water. The pH of the solution was adjusted to 8 by adding sodium bicarbonate and the aqueous layer was extracted with dichloromethane (3×20 mL). The combined organic extracts were dried over magnesium sulfate, filtered and concentrated in vacuo. Purification on preparative TLC of the residue (ethyl acetate/methanol/triethylamine, 8:1.5:0.5) gave [6-(4-methyl-[1,4]diazepan-1-yl)-pyridin-2-yl]-(R)-3-o-tolyloxy-pyrrolidin-1-yl)-methanone (31 mg, 16%) as a sticky residue. ¹H NMR (400 MHz, CDCl₃): δ 7.59-7.53 (dd, 1H), 7.20-7.11 (m, 3H), 6.91-6.75 (m, 2H), 6.57-6.52 (dd, 1H), 5.01 (br. s, 1H), 4.12-3.83 (m, 6H), 3.69-3.56 (m, 4H), 2.97-2.63 (m, 4H), 2.58-2.52 (2s, 3H), 2.32-2.08 (m, 4H), 2.20-2.16 (2s, 3H). HPLC (X5 Acidic): 92.84%. MS m/z 395 [C₂₃H₃₀N₄O₂+1].

### [6-(4-Methyl-[1,4]diazepan-1-yl)-pyridin-2-yl]-((R)-3-o-tolyloxy-pyrrolidin-1-yl)-methanone hydrochloride

To a solution of [6-(4-methyl-[1,4]diazepan-1-yl)-pyridin-2-yl]-((R)-3-o-tolyloxy-pyrrolidin-1-yl)-methanone (31 mg, 0.08 mmol) in CH₂Cl₂ (2 mL) was added 1 mL of HCl (1M in ether) dropwise at rt. The reaction mixture was stirred at room temperature for 1 hour, then was concentrated in vacuo. The residue was triturated in ether and dried by decantation to give [6-(4-methyl-[1,4]diazepan-1-yl)-pyridin-2-yl]-((R)-3-o-tolyloxy-pyrrolidin-1-yl)-methanone hydrochloride (25 mg, 74%) as a brown foam, mp=94-96° C. ¹H NMR (400 MHz, DMSO-d₆): δ 10.30 (br. s, 1H), 7.70-7.65 (dd, 1H), 7.19-6.79 (m, 6H), 5.11 (br. s, 1H), 4.00-3.39 (m, 10H), 3.17-3.01 (m, 2H), 2.82-2.77 (2s, 3H), 2.35-2.09 (m, 4H), 2.14-2.09 (2s, 3H). HPLC (X5 Acidic): 95.10%. MS m/z 395 [C₂₃H₃₀N₄O₂+1].

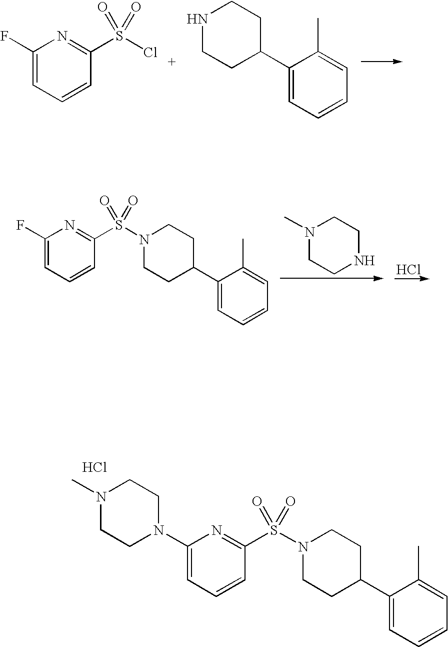

### EXAMPLE 9

### 2-Fluoro-6-(4-o-tolyl-piperidine-1-sulfonyl)-pyridine

A solution of 6-fluoro-pyridine-2-sulfonyl chloride (0.4 g, 2.05 mmol) in chloroform (10 mL) was cooled to 0° C., at which time 4-o-tolyl-piperidine (0.3 g, 1.42) in 10 mL of chloroform was added dropwise. The mixture was stirred for 10 min then quenched with water, extracted with chloroform, washed with water, brine, dried over sodium sulfate and concentrated. The residue was purified by column chromatography (100% CH₂Cl₂) to afford 2-fluoro-6-(4-o-tolyl-piperidine-1-sulfonyl)-pyridine as a white solid (0.18 g, 38%). ¹H NMR 400 MHz (CDCl₃) δ 8.05-8.00 (m, 1H), 7.90 (d, 1H), 7.20-7.10 (m, 5H), 4.10 (d, 2H), 3.00-2.90 (m, 2H), 2.82-2.75 (m, 1H), 2.35 (s, 3H), 1.90-1.80 (m, 4H).

### 1-Methyl-4-[6-(4-o-tolyl-piperidine-1-sulfonyl)-pyridin-2-yl]-piperazine

A mixture of 2-fluoro-6-(4-o-tolyl-piperidine-1-sulfonyl)-pyridine (0.18 g, 0.54 mmol), 1-methyl-piperazine (0.08 g, 0.81 mmol) In 3 mL of DMSO and 1,1,3,3 tetramethyl guanidine (0.124 g, 1.1 mmol) was heated at 80° C. overnight, the reaction mixture was cooled and extracted with ethyl acetate, washed with water, brine, dried over Na₂SO₄, and concentrated. The residue was purified by column chromatography (3% MeOH/CH₂Cl₂) to give 1-methyl-4-[6-(4-o-tolyl-piperidine-1-sulfonyl)-pyridin-2-yl]-piperazine as a white foam (0.2 g, 90%). ¹H NMR 400 MHz (CDCl₃) δ 7.62 (t, 1H), 7.28 (s, 1H), 7.20-7.10 (m, 4H), 6.80 (d, 1H), 4.08 (d, 2H), 3.68-3.60 (m, 4H), 2.88-2.70 ( m, 3H), 2.56-2.50 (m, 4H), 2.36 (s, 3H), 2.30 (s, 3H), 1.86-1.76 (m,4H).

### 1-Methyl-4-[6-(4-o-tolyl-piperidine-1-sulfonyl)-pyridin-2-yl]-piperazine hydrochloride

To a solution of 1-methyl-4-[6-(4-o-tolyl-piperidine-1-sulfonyl)-pyridin-2-yl]-piperazine (0.2 g, 0.48 mmol), in 5 mL of dichloromethane, 1 mL of 1 M HCl in ether was added dropwise. The reaction mixture was stirred at room temperature for 30 min, the reaction mixture was concentrated, washed with ether to afford 1-methyl-4-[6-(4-o-tolyl-piperidine-1-sulfonyl)-pyridin-2-yl]-piperazine hydrochloride as a light yellow solid (0.2 g, 88%). MS m/z 415 (M+1), HPLC 95.57%, mp. 203-204.5° C. ¹H NMR 400 MHz (DMSO-d₆) δ 7.88 (t, 1H), 7.25 (t, 2H), 7.18-7.04 (m, 4H), 4.45 (d, 2H), 3.85 (d, 2H), 3.60-3.45 (m, 2H) 3.40-3.35 (m, 2H), 3.20-3.00 (m, 2H), 2.85-2.75 (m, 6H), 2.25 (s, 3H), 1.75 (d, 2H), 1.70-1.60 (m, 2H).

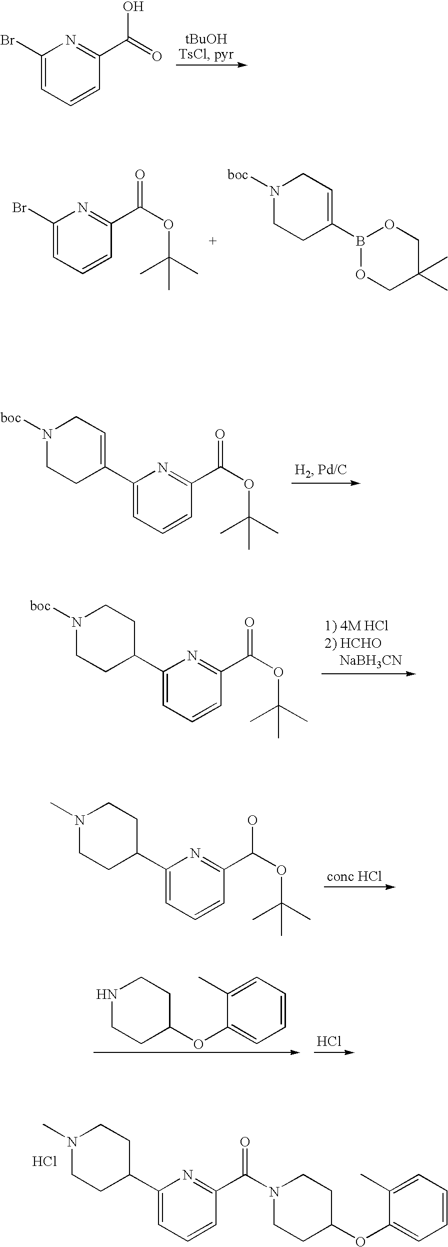

### EXAMPLE 10

### 6-Bromo-pyridine-2-carboxylic acid tert-butyl ester

To a solution of commercially available 6-bromo-pyridine-2-carboxylic acid (20 g, 100 mmol) in tert-butanol (500 mL) was added pyridine (70 mL) followed by TsCl (38.1 g, 200 mmol) at 0° C. After being stirred at ambient temperature for 3 h the reaction was quenched with sat. NaHCO₃ (aq) and stirred for 30 min (pH ˜8) at which time the solvent was concentrated to ca. 100 mL total volume. After filtration on a Buchner funnel white needles were collected and washed several times with water, then dried in vacuo over P₂O₅ to afford 6-bromo-pyridine-2-carboxylic acid tert-butyl ester (23.7 g, 93%) as white needles. ¹H NMR (400 MHz, CDCl₃) δ 8.03-8.01 (dd, 1H), 7.95-7.88 (m, 2H), 1.56 (s, 9H).

### 3′,6′-Dihydro-2′H-[2,4′]bipyridinyl-6,1′-dicarboxylic acid di-tert-butyl ester

A mixture of 6-bromo-pyridine-2-carboxylic acid tert-butyl ester (20.0 g, 77.5 mmol), tert-butyl-4-(5,5-dimethyl-1,3,2-dioxaborinan-2-yl)-5,6-dihydropyridine-1(2H)-carboxylate (22.8 g, 77.5 mmol) and cesium fluoride (17.6 g, 116 mmol) in N,N-dimethylformamide (240 mL) was degassed for 30 min, and [1,1′-bis(diphenylphosphino)ferrocene] palladium (II) (8.5 g, 10.4 mmol) was added. The reaction mixture was heated at 100° C. overnight, cooled, and concentrated. EtOAc was added and the suspension filtered through a pad of Celite. The filtrate was washed with water, dried (MgSO₄), and concentrated. The crude product was purified by flash chromatography on silica gel using hexanes/ethyl acetate 5:1, to give 3′,6′-dihydro-2′H-[2,4′]bipyridinyl-6,1′-dicarboxylic acid di-tert-butyl ester (21.4 g, 76%) as an orange oil. ¹H NMR (400 MHz, CDCl₃): δ 7.90 (dd, 1H), 7.80-7.75 (dd, 1H), 7.51 (m, 1H), 6.80-6.70 (br. s, 1H), 4.18 (br. s, 1H), 3.65 (br. s, 1H), 2.70 (br. s, 1H), 1.62 (s, 9H), 1.48 (s, 9H). MS m/z 361 [C₂₀H₂₈N₂O₄+1].

### 3′,4′,5′,6′-Tetrahydro-2′H-[2,4′]bipyridinyl-6,1′-dicarboxylic acid di-tert-butyl ester

To a solution of 3′,6′-dihydro-2′H-[2,4′]bipyridinyl-6,1′-dicarboxylic acid di-tert-butyl ester (10.0 g, 27.7 mmol) in methanol (100 mL) was added 10% palladium on carbon (˜4 g). The reaction mixture was stirred overnight at ambient temperature under 50 psi of hydrogen gas. It was then filtered through a pad of Celite, and the filtrate concentrated to afford 3′,4′,5′,6′-Tetrahydro-2′H-[2,4′]bipyridinyl-6,1′-dicarboxylic acid di-tert-butyl ester (9.33 g, 93%) as a green oil which was used in the next step without further purification. ¹H NMR (400 MHz, CDCl₃): δ 7.88 (dd, 1H), 7.75 (dd, 1H), 7.30 (dd, 1H), 4.20 (br. s, 1H), 3.01 (m, 1H), 2.82 (br. s, 1H), 1.98 (m, 2H), 1.70 (m, 2H), 1.61 (s, 9H), 1.43 (s, 9H). MS m/z 363 [C₂₀H₃₀N₂O₄1].

### 1′,2′,3′,4′,5′,6′-Hexahydro-[2.4′]bipyridinyl-6-carboxylic acid tert-butyl ester

A solution of 4M HCl in dioxane (100 mL) was added to 3′,4′,5′,6′-tetrahydro-2′H-[2,4′]bipyridinyl-6,1′-dicarboxylic acid di-tert-butyl ester (9.3 g, 25.66 mmol) at 0° C. The mixture was stirred at ambient temperature for 10 min, and sat. NaHCO₃ (aq) was added until pH 8 was reached. Water (100 mL) was added and the mixture was extracted with CH₂Cl₂ (3×200 mL), dried (MgSO₄), and concentrated to afford 1′,2′,3′,4′,5′,6′-Hexahydro-[2,4′]bipyridinyl-6-carboxylic acid tert-butyl ester (4.8 g, 71%) which was used without further purification. ¹H NMR (400 MHz, CDCl₃) δ 8.28 (br. s, 1H), 7.86 (dd, 1H), 7.78 (dd, 1H), 7.39 (dd, 1H), 3.55 (m, 2H), 3.18 (m, 1H), 3.03 (m, 2H), 2.18 (br. s, 4H), 1.58 (s, 9H). MS m/z 263 [C₁₅H₂₂N₂O₂+1].

### 1′-Methyl-1′,2′,3′,4′,5′,6′-hexahydro-[2,4′]bipyridinyl-6-carboxylic acid tert-butyl ester

A mixture of 1′,2′,3′,4′,5′,6′-Hexahydro-[2,4′]bipyridinyl-6-carboxylic acid tert-butyl ester (4.8 g, 18.3 mmol) and formaldehyde (37% in water, 8.7 mL, 73.2 mmol) in methanol (50 mL) was stirred at ambient temperature for 15 min. Sodium cyanoborohydride (4.6 g, 73.2 mmol) was added and the mixture stirred for 30 min. The reaction was quenched with water and extracted with CH₂Cl₂, dried (MgSO₄), and concentrated to provide 1′-methyl-1′,2′,3′,4′,5′,6′-hexahydro-[2,4′]bipyridinyl-6-carboxylic acid tert-butyl ester (4.68 g, 92%) which was used without further purification. ¹H NMR (400 MHz, CDCl₃) δ 7.85 (dd, 1H), 7.78 (dd, 1H), 7.37 (dd, 1H), 3.18 (m, 2H), 2.98 (m, 1H), 2.43 (s, 3H), 2.32 (m, 2H), 2.09 (m, 2H), 1.92 (m, 2H), 1.61 (s, 9H). MS m/z 276 [C₁₆H₂₄N₂O₂+1].

### 1′-Methyl-1′,2′,3′,4′,5′,6′-hexahydro-[2,4′]bipyridinyl-6-carboxylic acid

A solution of 1′-methyl-1′,2′,3′,4′,5′,6′-hexahydro-[2,4′]bipyridinyl-6-carboxylic acid tert-butyl ester (4.68 g, 16.9 mmol) in concentrated hydrochloric acid (50 mL) was stirred at ambient temperature for 4 h. The mixture was concentrated and the residue dissolved in the minimum amount of MeOH. Et₂O was added until precipitation of a solid which was collected by filtration and dried to give 1′-methyl-1′,2′,3′,4′,5′,6′-hexahydro-[2,4′]bipyridinyl-6-carboxylic acid hydrochloride (4.92 g, quantitative) as a beige solid which was used without further purification. ¹H NMR (400 MHz, DMSO-d₆) δ 10.41 (br. s, 1H), (dd, 1H), 8.00-7.91 (m, 2H), 7.55 (dd, 1H), 7.42 (d, 1H mobile), 7.33 (d, 1H mobile), 7.18 (d, 1H mobile), 3.50 (m, 2H), 3.15-3.02 (m, 3H), 2.78 (2s, 3H), 2.18-2.02 (m, 4H). MS m/z 221 [C₁₂H₁₆N₂O₂+1].

### (1′-Methyl-1′,2′,3′,4′,5′,6′-hexahydro-[2,4′]bipyridinyl-6-yl)-(4-o-tolyloxy-piperidin-1-yl)-methanone

A mixture of 1′-methyl-1′,2′,3′,4′,5′,6′-hexahydro-[2,4′]bipyridinyl-6-carboxylic acid hydrochloride (0.35 g, 1.36 mmol), 4-o-tolyloxy-piperidine hydrochloride (0.26 g, 1.14 mmol), diisopropylethylamine (1.6 mL, 9.09 mmol) and HATU (0.61 g, 1.59 mmol) in DMF (10 mL) was stirred overnight at ambient temperature. The mixture was diluted with EtOAc (10 mL) and the organic phase separated and washed with brine (3×50 mL), dried (MgSO₄), and concentrated. Silica gel chromatography (CH₂Cl₂/MeOH, 95:5) of the residue gave (1′-methyl-1′,2′,3′,4′,5′,6′-hexahydro-[2,4′]bipyridinyl-6-yl )-(4-o-tolyloxy-piperidin-1-yl)-methanone (0.12 g, 27%) as an amber foam. ¹H NMR (400 MHz, CDCl₃) δ 7.78 (m, 1H), 7.42 (m, 1H), 7.22 (m, 1H), 7.12 (m, 2H), 6.83 (m, 2H), 4.64 (br. s, 1H), 4.03 (m, 1H), 3.75 (m, 1H), 3.59 (m, 1H), 3.41 (m, 3H), 3.01 (m, 3H), 2.80 (s, 3H), 2.21 (s, 3H), 2.10-1.82 (m, 8H). HPLC (X5 Acidic): 95.82%. MS m/z 394 [C₂₄H₃₁N₃O₂+1].

### (1′-Methyl-1′,2′,3′,4′,5′,6′-hexahydro-[2,4′]bipyridinyl-6-yl)-(4-o-tolyloxy-piperidin-1-yl)-methanone hydrochloride

To a solution of (1′-methyl-1′,2′,3′,4′,5′,6′-hexahydro-[2,4′]bipyridinyl-6-yl)-(4-o-tolyloxy-piperidin-1-yl)-methanone (0.12 g, 0.31 mmol) in CH₂Cl₂ (4 mL) was added dropwise 2 mL of HCl (1M in ether) at ambient temperature. The mixture was stirred for 1 h and concentrated. The residue was triturated with ether, filtered and dried to give (1′-methyl-1′,2′,3′,4′,5′,6′-hexahydro-[2,4′]bipyridinyl-6-yl)-(4-o-tolyloxy-piperidin-1-yl)-methanone hydrochloride (0.11 g, 84%) as an amber foam: HPLC (X5 Acidic): 95.8%, MS m/z 394 [C₂₄H₃₁N₃O₂+1], mp=118-120° C. ¹H NMR (400 MHz, DMSO-d₆): δ 7.91 (dd, 1H), 7.48 (dd, 1H), 7.40 (dd, 1H), 7.15 (m, 2H), 7.00 (dd, 1H), 6.82 (dd, 1H), 4.65 (m, 1H), 3.87 (m, 1H), 3.70-3.47 (m, 4H), 3.39 (m, 1H), 3.16-2.96 (m, 3H), 2.79 (s, 3H), 2.19 (s, 3H), 2.14-1.92 (m, 6H), 1.81-1.63 (m, 2H). Anal. Calcd for C₂₄H₃₁N₃O₂ 2.2HCl.2CH₂Cl₂:C, 48.52; H, 5.83; N, 6.53; Found: C, 48.40; H, 5.85; N, 6.96.

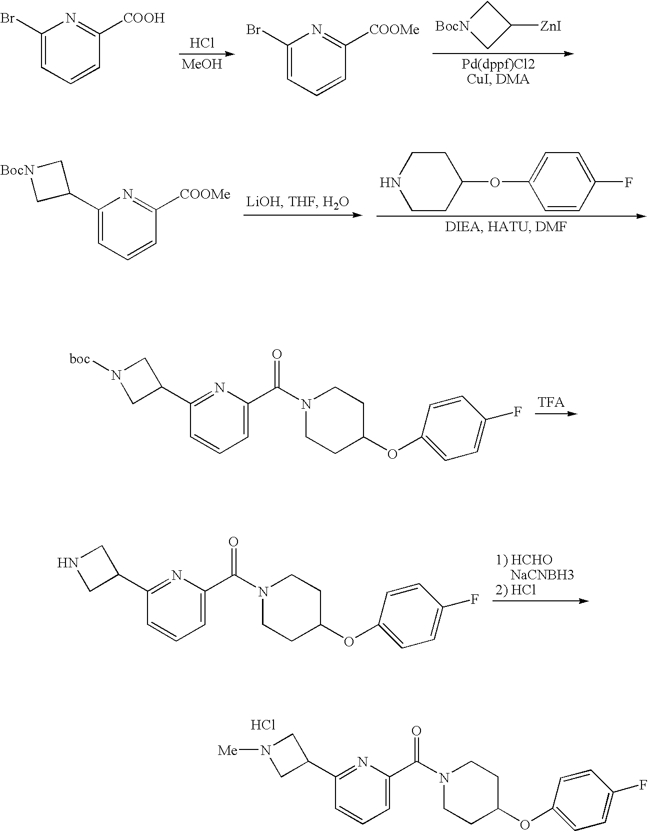

### EXAMPLE 11

### 6-Bromo-pyridine-2-carboxylic acid methyl ester

To a solution of 6-bromo-pyridine-2-carboxylic acid (20.0 g, 99.5 mmol) in MeOH (200 mL) was added conc. HCl (10 mL) dropwise. The reaction mixture was stirred at room temperature for 5 h, concentrated, and the residue basified with saturated NaHCO₃. The mixture was extracted with EtOAc, washed with water, brine, dried (Na₂SO₄), and concentrated to afford 6-bromo-pyridine-2-carboxylic acid methyl ester (13.8 g, 64%) as a white solid. ¹H NMR (400 MHz, CDCl₃): δ 8.15 (d, 1H), 7.70 (m, 2H), 4.00 (s, 3H).

### [1-[(1,1-dimethylethoxy)carbonyl]-3-azetidinyl]iodo-zinc

A 25 mL round bottom flask was charged with 180 mg of celite and dried in oven for 1 hour then cooled under vacuum. DMA (2.32 mL) and Zinc dust (0.78 g, 12.0 mmol) were added. While stirring under nitrogen, TMSCl (0.12 g, 1.1 mmol) and dibromoethane (0.21 g, 1.1 mmol) were added dropwise simultaneously over 10 minutes, maintaining the internal temperature not more than 40° C. After addition the mixture was stirred at room temperature for 30 minutes. A solution of 3-iodo-azetidine-1-carboxylic acid tert-butyl ester (2.83 g, 10.0 mmol) in DMA (4.64 mL) was added dropwise to keep internal temperature below 40° C., then stirred at room temperature for 2 hour to give [1-[(1,1-dimethylethoxy)carbonyl]-3-azetidinyl]iodo-zinc as 1.0 M solution in DMA, that was used in the next step immediately.

### 6-(1-tert-butoxycarbonyl-azetidin-3-yl)-pyridine-2-carboxylic acid methyl ester

A solution of [1-[(1,1-dimethylethoxy)carbonyl]-3-azetidinyl]iodo-zinc (1.0M soln in DMA, 25 mL, 25.0 mmol) was added to a mixture of 6-bromo-pyridine-2-carboxylic acid methyl ester (8.83 g, 40.7 mmol), Pd(dppa)Cl₂ CH₂Cl₂ (1.35 g, 1.85 mmol), and CuI (0.7 g, 3.7 mmol) in DMA (20 mL). The mixture was degassed with nitrogen for 10 min, then heated at 80° C. overnight. The mixture was cooled to room temperature and diluted with ethyl acetate (50 mL). The organic layer was washed with 1N NH₄Cl (10 mL), brine (2×10 mL), dried (Na₂SO₄), and concentrated in vacuo. Silica gel chromatography (hexane/ethyl acetate, 1:1) gave 6-(1-tert-butoxycarbonyl-azetidin-3-yl)pyridine-2-carboxylic acid methyl ester (6.5 g, 49%) as a light yellow solid. ¹H NMR (400 MHz, CDCl₃): δ 8.05 (d, 2H), 7.90 (t, 2H), 7.60 (d, 2H), 4.20 (t, 2H), 4.10 (t, 2H), 4.05 (m, 1H), 4.00 (s, 3H), 1.42 (s, 9H).

### 6-(1-tert-butoxycarbonyl-azetidin-3-yl)-pyridine-2-carboxylic acid

To a solution of 6-(1-tert-butoxycarbonyl-azetidin-3-yl)-pyridine-2-carboxylic acid methyl ester (6.2 g, 0.02 mol) in 60 mL of THF and 12 mL of water was added LiOH.H₂O (4.45 g, 0.10 mol). The reaction mixture was stirred at room temperature overnight. The pH was adjusted to 5˜6 with 1N HCl. The reaction mixture was concentrated and the residue was extracted with dichloromethane, washed with water, brine, dried (Na₂SO₄) and concentrated to afford 6-(1-tert-butoxycarbonyl-azetidin-3-yl)-pyridine-2-carboxylic acid as a brown solid (4.7 g, 85%). ¹H NMR 400 MHz (CDCl₃) δ 8.18 (d, 1H), 7.95 (t, 1H), 7.55 (d, 1H), 4.38 (t, 2H), 4.18 (t, 2H), 4.00-3.90 (m, 1H).

### 3-{6-[4-(4-Fluoro-phenoxy)-piperidine-1-carbonyl]-pyridin-2-yl}-azetidine-1-carboxylic acid tert-butyl ester

A mixture of 6-(1-tert-butoxycarbonyl-azetidin-3-yl)-pyridine-2-carboxylic acid (0.35 g, 1.26 mmol), 4-(4-fluoro-phenoxy)-piperidine (0.32 g, 1.38 mmol) in 5 mL of DMF and diisopropylethylamine (0.358 g, 2.77 mmol), 2-(7-aza-1H-benzotriazole-1-yl)-1,1,3,3-tetramethyl uronium hexafluorophosphate (HATU) (0.52 g, 1.38 mmol) was stirred at room temperature overnight, extracted with ethyl acetate, washed with saturated NaHCO₃, water, brine, dried over sodium sulfate and concentrated. The residue was purified by column chromatography (50-75% EtOH/Hexane) to furnish 3-{6-[4-(4-fluoro-phenoxy)-piperidine-1-carbonyl]-pyridin-2-yl}-azetidine-1-carboxylic acid tert-butyl ester as a white foam (0.36 g, 62.8%). ¹H NMR 400 MHz (CDCl₃) δ 7.75 (t, 1H), 7.58 (d 1H), 7.22 (d, 1H), 6.98 (t, 2H), 6.90-6.85 (m, 2H), 4.50 (m, 1H), 4.30 (t, 2H), 4.15 (t. 2H), 3.90-3.75 (m, 4H), 3.60-3.52 (m, 1H), 2.10-1.82 (m, 4H), 1.45 (s, 9H).

### (6-Azetidin-3-yl-pyridin-2-yl)-[4-(4-fluoro-phenoxy)-piperidin-1-yl]-methanone

A solution of 3-{6-[4-(4-fluoro-phenoxy)-piperidine-1-carbonyl]-pyridin-2-yl}-azetidine-1-carboxylic acid tert-butyl ester (0.35 g, 0.76 mmol) in 10 mL of dichloromethane and 2 mL of trifluoroacetic acid was stirred at room temperature overnight. Saturated NaHCO₃ was added, and the mixture was extracted with dichloromethane, washed with water, brine, dried (Na₂SO₄) and concentrated to give (6-azetidin-3-yl-pyridin-2-yl)-[4-(4-fluoro-phenoxy)-piperidin-1-yl]-methanone as a white sticky (0.275 g, quantitative). ¹H NMR 400 MHz (CDCl₃) δ 7.80 (t, 1H), 7.58 (d, 1H), 7.22 (d, 1H), 6.98 (t, 2H), 6.90-6.80 (m, 2H), 4.60 (m, 1H), 4.50-4.30 (m, 5H), 4.00-3.85 (m, 2H), 3.75 (m, 1H), 3.55 (m, 1H), 2.10-2.02 (m, 2H), 2.00-85 (m, 2H).

### [4-(4-Fluoro-phenoxy)-piperidin-1-yl]-[6-(1-methyl-azetidin-3-yl)-pyridin-2-yl]-methanone

A solution of (6-azetidin-3-yl-pyridin-2-yl)-[4-(4-fluoro-phenoxy)-piperidin-1-yl]-methanone (0.2 g, 0.56 mmol) in 10 mL of methanol and 0.17 mL of formaldehyde was stirred at room temperature for 15 min. Sodium cyanoborohydride (0.14 g, 2.25 mmol) was added to the reaction mixture and again stirred for 30 min at room temperature, quenched with water, extracted with dichloromethane, washed with water, brine, dried over sodium sulfate and concentrated. The residue was purified by column chromatography (5% MeOH, 1% NH₄OH in dichloromethane) to afford [4-(4-fluoro-phenoxy)-piperidin-1-yl]-[6-(1-methyl-azetidin-3-yl)-pyridin-2-yl]-methanone as a clear sticky material (0.14 g, 68%). ¹H NMR 400 MHz (CDCl₃) δ 7.72 (t, 1H), 7.50 (d, 1H), 7.30 (d, 1H), 7.00 (t, 2H), 6.90-6.85 (m, 2H), 4.52 (m, 1H), 4.00-3.92 (m, 1H), 3.90-3.75 (m, 5H), 3.60-3.52 (m, 1H), 3.38 (t, 2H), 2.40 (s, 3H), 2.10-1.85 (m, 4H).

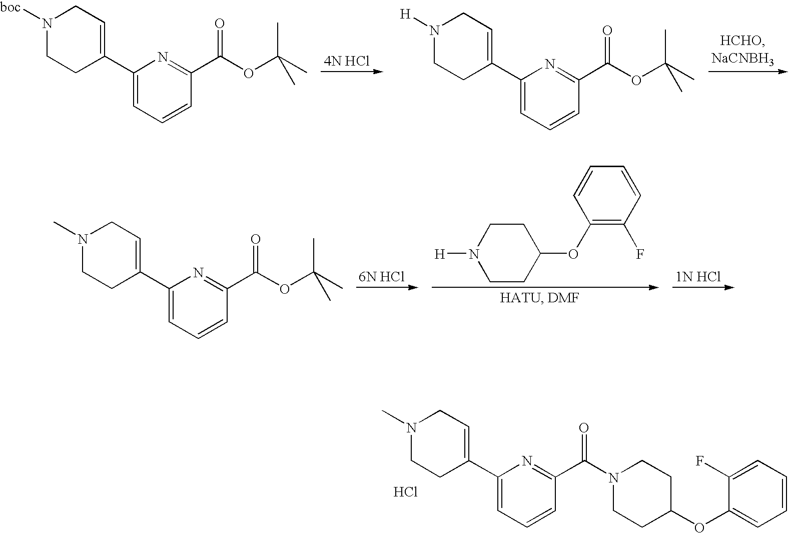

### EXAMPLE 12

### 1′,2′,3′,6′-Tetrahydro-[2,4′]bipyridinyl-6-carboxylic acid tert-butyl ester

To a solution of 3′,6′-dihydro-2′H-[2,4′]bipyridinyl-6,1′-dicarboxylic acid di-tert-butyl ester (1.00 g, 2.78 mmol) in anhydrous dioxane (5 mL) at 0° C. was added hydrochloric acid (10.0 mL, 40 mmol, 4 M solution in dioxane). The mixture was warmed to ambient temperature and stirred 25 min, then cooled to 0° C. and neutralized with sat. NaHCO₃ (aq). The mixture was extracted with CH₂Cl₂ (3×25 mL) and the combined organic phases dried (Na₂SO₄), and concentrated to afford 1′,2′,3′,6′-tetrahydro-[2,4′]bipyridinyl-6-carboxylic tert-butyl ester which was used without further purification (0.80 g). ¹H NMR (400 MHz, CD₃OD) δ 8.03 (m, 2H), 7.86 (m, 1H), 6.75 (m, 1H), 3.92 (m, 2H), 3.50 (m, 2H), 2.97 (m, 2H), 1.66 (s, 9H). MS (ES) 261.01 [C₁₅H₂₀N₂O₂+H]⁺.

### 1′-Methyl-1′,2′,3′,6′-tetrahydro-[2,4′]bipyridinyl-6-carboxylic acid tert-butyl ester

To a solution of 1′,2′,3′,6′-tetrahydro-[2,4′]bipyridinyl-6-carboxylic acid tert-butyl ester (0.80 g, 3.08 mmol) in methanol (10 mL) was added formaldehyde (1.37 mL, 12.30 mmol, 37% solution in water). The mixture was stirred 20 min at which time sodium cyanoborohydride (0.772 g, 12.3 mmol) was added in one portion. The mixture was stirred for 1 h and then quenched by addition of water (5 mL). The volatiles were removed under reduced pressure and the residue was extracted with CH₂Cl₂ (3×20 mL). The organic extracts were dried (Na₂SO₄), concentrated, and purified by flash chromatography (10% methanol in CH₂Cl₂) to provide 1′-methyl-1′,2′,3′,6′-tetrahydro-[2,4′]bipyridinyl-6-carboxylic acid tert-butyl ester (0.31 g, 41%) as a gummy solid. ¹H NMR (400 MHz, CDCl₃) δ 7.86 (dd, 1H), 7.73 (t, 1H), 7.49 (dd, 1H), 6.77 (m, 1H), 3.19 (AB q, 2H), 2.75 (m, 2H), 2.70 (m, 2H), 2.42 (s, 3H), 1.63 (s, 9H). MS (ES) 275.07 [C₁₆H₂₂N₂O₂+H]⁺.

### 1′-Methyl-1′,2′,3′,6′-tetrahydro-[2,4′]bipyridinyl-6-carboxylic acid hydrochloride

To a suspension of 1′-methyl-1′,2′,3′,6′-tetrahydro-[2,4′]bipyridinyl-6-carboxylic acid tert-butyl ester (0.31 g, 1.13 mmol) in water (1 mL) at 0° C. was added conc. hydrochloric acid (6 mL), and then the mixture was stirred at ambient temperature for 4 h. The volatiles were removed under reduced pressure to give 1′-methyl-1′,2′,3′,6′-tetrahydro-[2,4′]bipyridinyl-6-carboxylic acid hydrochloride as a brown solid (quantitative yield) which was used without further purification. ¹H NMR (400 MHz, CD₃OD) δ 8.35 (m, 1H), 8.30 (m, 1H), 8.10 (m, 1H), 6.72 (m, 1H), 4.20 (m, 1H), 3.92 (m, 1H), 3.80 (m, 1H), 3.46 (m, 1H), 3.10 (m, 2H), 3.06 (s, 3H); MS (ES) 219.04 [C₁₂H₁₄N₂O₂+H]⁺.

### [4-(2-Fluoro-phenoxy)-piperidin-1-yl]-(1′-methyl-1′,2′,3′,6′-tetrahydro-[2,4′]bipyridinyl-6-yl)-methanone

To a solution of 1′-methyl-1′,2′,3′,6′-tetrahydro-[2,4′]bipyridinyl-6-carboxylic acid hydrochloride (0.29 g, 1.13 mmol) in anhydrous DMF (10 mL) was added 4-(2-fluoro-phenoxy)-piperidine hydrochloride (0.26 g, 1.13 mmol), O-(7-azabenzotriazol-1-yl N,N,N′,N′-tetramethyluronium hexafluorophosphate (HATU) (0.60 g, 1.58 mmol), and diisopropylethylamine (1.57 mL, 9.04 mmol) and the whole stirred at ambient temperature overnight. The mixture was diluted with water and extracted with ethyl acetate (2×25 mL). The organic phase was separated and washed with water (2×20 mL), brine (20 mL), dried, concentrated, and purified by flash chromatography (5% MeOH/CH₂Cl₂) to furnish [4-(2-fluoro-phenoxy)-piperidin-1-yl]-(1′-methyl-1′,2′,3′,6′-tetrahydro-[2,4′]bipyridinyl-6-yl)-methanone as an oil (70 mg). ¹H NMR (400 MHz, CD₃OD) δ 7.90 (m, 1H), 7.66 (m, 1H), 7.46 (m, 1H), 7.23-7.00 (m, 3H), 6.94 (m, 1H), 6.73 (m, 1H), 4.66 (m, 1H), 3.97 (m, 1H), 3.77 (m, 2H), 3.50 (m, 1H), 3.30 (m, 2H), 2.77 (m, 4H), 2.46 (m, 3H), 2.06 (m, 2H), 1.86 (m, 2H); ¹⁹F NMR (400 MHz, CD₃OD) δ −136. MS (ES) 396.19 [C₂₃H₂₆FN₃O₂+H]⁺. HPLC purity (95.1%).

### [4-(2-Fluoro-phenoxy)-piperidin-1-yl]-(1′-methyl-1′,2′,3′,6′-tetrahydro-[2,4′]bipyridinyl-6-yl)-methanone hydrochloride

To a solution of [4-(2-fluoro-phenoxy)-piperidin-1-yl]-(1′-methyl-1′,2′,3′,6′-tetrahydro-[2,4′]bipyridinyl-6-yl)-methanone (70 mg) in anhydrous dichloromethane (3 mL) at 0° C. was added hydrochloric acid (0.5 mL, 0.5 mmol, 1M solution in diethyl ether). The mixture was stirred for 1 h and concentrated to give [4-(2-fluoro-phenoxy)-piperidin-1-yl]-(1′-methyl-1′,2′,3′,6′-tetrahydro-[2,4′]bipyridinyl-6-yl)-methanone hydrochloride (70 mg) as a pale yellow solid: MS (ES) 396.06 [C₂₃H₂₆FN₃O₂+H]⁺; HPLC purity (96.3%). ¹H NMR (400 MHz, CD₃OD) δ 7.96 (m, 1H), 7.75 (d, 1H), 7.55 (d, 1H), 7.20-7.06 (m, 3H), 6.96 (m, 1H), 6.74 (m, 1H), 4.68 (m, 1H), 4.13 (m, 1H), 4.00 (m, 1H), 3.88 (m, 1H), 3.78 (m, 3H), 3.48 (m, 1H), 3.36 (m, 1H), 3.16-2.90 (m, 5H), 2.08 (m, 2H), 1.90 (m, 2H); ¹⁹F NMR (400 MHz, CD₃OD) δ −136. Anal. Calcd for C₂₃H₂₆FN₃O₂.2HCl.H₂O % C, 56.79, H, 6.22, N, 8.64; Found: C, 57.13, H, 6.08, N, 8.87.

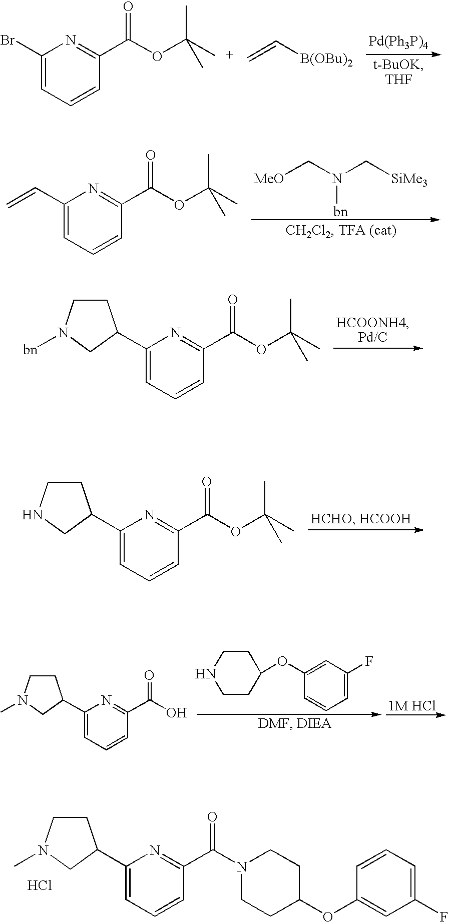

### EXAMPLE 13

### 6-Vinyl-pyridine-2-carboxylic acid tert-butyl ester

A mixture of 6-bromo-pyridine-2-carboxylic acid tert-butyl ester (10.0 g, 38.7 mmol), vinylboronic acid dibutyl ester (8.55 g, 46.5 mmol), and potassium tert-butoxide (5.21 g, 46.5 mmol) in THF (100 mL) was degassed for 30 minutes, at which time tetrakis(triphenylphosphine)palladium(0) (2.23 g, 1.93 mmol) was added. The mixture was heated at 100° C. overnight, cooled to ambient temperature, and concentrated. EtOAc was added and the suspension filtered through a pad of Celite. The filtrate was washed with water, dried (MgSO₄), and concentrated. The crude product was purified by flash chromatography on silica gel using hexanes/ethyl acetate 5:1, to give 6-vinyl-pyridine-2-carboxylic acid tert-butyl ester (6.2, 78%) as an orange oil. ¹H NMR (400 MHz, CDCl₃): δ 7.90 (d, 1 H), 7.79 (dd, 1H), 7.50 (d, 1H), 7.00-6.90 (m, 1H), 6.28 (d, 1H), 5.38 (d, 1H), 1.60 (s, 9H). MS (ES⁺) m/z 205.26 (M+1).

### 6-(1-Benzyl-pyrrolidin-3-yl)-pyridine-2-carboxylic acid tert-butyl ester

To a solution of 6-vinyl-pyridine-2-carboxylic acid tert-butyl ester (2.0 g, 9.74 mmol) and N-(methoxymethyl)-N-(trimethylsilylmethyl)benzylamine (2.77 g, 3.0 mmol) in CH₂Cl₂ (20 mL) was added TFA (0.1 mL). The reaction mixture was stirred for 2 h and then quenched with water and basified with solid NaHCO₃. Organic materials were separated and dried (MgSO₄) After filtration and evaporation of the solvent the crude material was purified by silica gel column chromatography using 5% MeOH with 1% NH₄OH in CH₂Cl₂ to afford 6-(1-benzyl-pyrrolidin-3-yl)-pyridine-2-carboxylic acid tert-butyl ester (2.8 g, 85%) as a brown liquid. ¹H NMR (400 MHz, CDCl₃): δ 7.81 (d, 1H), 7.78-7.70 (dd, 1H), 6.56 (d, 1H), 7.40-7.20 (m, 5H), 3.78-3.62 (m, 3H), 3.10-2.98 (m, 1H), 2.90-2.70 (m, 3H), 2.44-2.39 (m, 1H), 2.10-2.00 (m, 1H), 1.62 (s, 9H). MS (ES⁺) m/z 339.16 (M+1).

### 6-Pyrrolidin-3-yl-pyridine-2-carboxylic acid tert-butyl ester

A mixture of 6-(1-benzyl-pyrrolidin-3-yl)-pyridine-2-carboxylic acid tert-butyl ester (2.8 g, 8.27 mmol), ammonium formate (7.8 g, 8.27 mmol), and Pd/C (2.0 g, 10 wet %) in MeOH (50 mL) was refluxed under nitrogen for 2 h. The solid catalyst was removed by filtration through Celite. The filtrate was purified by silica gel column chromatography using 10% MeOH, & 1% ammonium hydroxide in CH₂Cl₂ to afford 6-pyrrolidin-3-yl-pyridine-2-carboxylic acid tert-butyl ester (1.8 g, 88%) as a brown liquid. ¹H NMR (400 MHz, CDCl₃): δ 7.90-7.80 (m 1H), 7.78-7.70 (m, 1H), 6.39-6.30 (m, 1H), 3.60-3.50 (m, 1H), 3.38-3.00 (m, 3H), 2.40-1.98 (m, 3H), 1.60 (s, 9H). MS (ES⁺) m/z 249.10 (M+1).

### 6-(1-Methyl-pyrrolidin-3-yl)-pyridine-2-carboxylic acid

A mixture of 6-pyrrolidin-3-yl-pyridine-2-carboxylic acid tert-butyl ester (1.8 g, 7.2 mmol), formaldehyde (2.0 ml, 37% in water), & formic acid (2.0 mL) was heated to 100° C. for 15 min, cooled and quenched with water and basified with solid NaHCO₃. The aqueous layer was then extracted with with EtOAc to remove the impurities. Evaporation of the water under reduced pressure afforded the crude solid which was then triturated with MeOH and filtered. Evaporation of the filtrate afforded the target compound 6-(1-methyl-pyrrolidin-3-yl)-pyridine-2-carboxylic acid (1.7 g, crude) which was used without further purification. ¹H NMR (400 MHz, DMSO-d₆): δ 7.82-7.79 (m 1H), 7.70-7.60 (m, 1H), 7.24-7.20 (m, 1H), 3.58-3.44 (m, 1H), 3.01-2.80 (m, 2H), 2.50-2.10 (m, 6H), 1.81-1.70 (m, 1H). MS (ES⁺) m/z 206.25 (M+1).

### [4-(3-Fluoro-phenoxy)-piperidin-1-yl]-[6-(1-methyl-pyrrolidin-3-yl)-pyridin-2-yl]-methanone

A mixture of 6-(1-methyl-pyrrolidin-3-yl)-pyridine-2-carboxylic acid monohydrate (0.22 g, 0.97 mmol, crude), 4-(3-fluoro-phenoxy)-piperidine hydrochloride (0.19 g, 0.80 mmol), diisopropylethylamine (0.50 mL, 2.91 mmol) and HATU (0.44 g, 1.16 mmol) in DMF (5 mL) was stirred overnight at ambient temperature. The mixture was diluted with EtOAc (10 mL), and the organic phase was separated and washed with brine (3×50 mL), dried (MgSO₄), and concentrated. Silica gel chromatography (CH₂Cl₂/MeOH, 95:5, with 1% NH₄OH) of the residue gave [4-(3-fluoro-phenoxy)-piperidin-1-yl]-[6-(1-methyl-pyrrolidin-3-yl)-pyridin-2-yl]-methanone (90 mg, 23%) as a white foam. ¹H NMR (400 MHz, CDCl₃): δ 7.79-7.70 (m, 1H), 7.58-7.52 (m, 1H), 7.30-7.20 (m, 2H), 6.80-6.62 (m, 3H), 4.65-4.60 (m, 1H), 4.00-3.50 (m, 5H), 3.30-3.18 (m, 1H), 3.08-2.97 (m, 1H), 2.80-2.68 (m, 2H), 2.50 (s, 3H), 2.45-2.38 (m, 1H), 2.19-1.90 (m, 5H). MS (ES⁺) m/z 384.09 (M+1). [4-(3-Fluoro-phenoxy)-piperidin-1-yl]-[6-(1-methyl-pyrrolidin-3-yl)-pyridin-2-yl]-methanone hydrochloride

To a solution of [4-(3-fluoro-phenoxy)-piperidin-1-yl]-[6-(1-methyl-pyrrolidin-3-yl)-pyridin-2-yl]-methanone (90 mg, 0.23 mmol) in CH₂Cl₂ (5 mL) was added 2.0 mL of HCl (1M in ether) dropwise at ambient temperature. The mixture was stirred for 15 min and concentrated. The residue was washed with hexanes and dried under vacuum to give [4-(3-fluoro-phenoxy)-piperidin-1-yl]-[6-(1-methyl-pyrrolidin-3-yl)-pyridin-2-yl]-methanone hydrochloride (85 mg) as a light yellow solid: HPLC: 96.5%, MS (ES⁺) m/z 384.15 (M+1), mp=85-86° C. ¹H NMR (400 MHz, DMSO-d₆): δ 11.30 (br s, 1H), 10.98 (br s, 1H), 8.06-7.99 (m, 1H), 7.60-7.50 (m, 2H), 7.41-7.30 (m, 1H), 7.00-6.79 (m, 3H), 4.82-4.78 (m, 1H), 4.18-3.20 (m, 9H), 2.98 (s, 3H), 2.50-1.82 (6H). Anal. Calcd for C₂₂H₂₆FN₃O₂ 1.5 HCl. 1.0H₂O: C, 57.93%; H, 6.52%; N, 9.21%; Found: C, 58.00%; H, 6.73%; N, 9.16%.

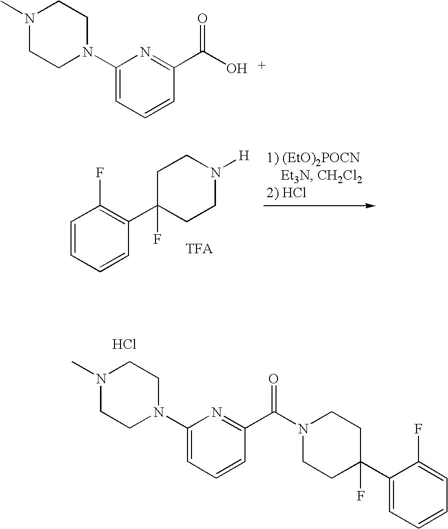

### EXAMPLE 14

### [4-Fluoro-4-(2-fluoro-phenyl)-piperidin-1-yl]-[6-(4-methyl-piperazin-1-yl)-pyridin-2-yl]-methanone hydrochloride

To a solution of 4-fluoro-4-(2-fluoro-phenyl)-piperidine, TFA salt (˜0.67 mmol), 6-(4-methyl-piperazin-1-yl)-pyridine-2-carboxylic acid (0.358 g, 1.0 mmol), and 0.7 mL of triethylamine in 20 mL of dichloromethane was added diethyl cyanophosphonate (0.13 g, 8 mmol) at room temperature. After addition, the mixture was stirred at room temperature for 1 h, then purified by chromatography (2.5-10% of methanol in dichloromethane) to give compound 7 with some Et₃N.TFA salt. This mixture was dissolved in 20 mL of dichloromethane and treated with 1 mL of HCl/dioxane (4M solution) at room temperature for 1 h. The solid was collected by filtration and washed with dichloromethane to give [4-fluoro4-(2-fluoro-phenyl)-piperidin-1-yl]-[6-(4-methyl-piperazin-1-yl)-pyridin-2-yl]-methanone hydrochloride as a solid (0.25 g, 70%). HPLC: 97.05%; MS m/n=401 [MH]⁺. ¹H NMR (400 MHz, CDCl₃): δ 7.81 (dd, 1H); 7.56 (dd, 1H); 7.40-7.10 (m, 4H); 7.03 (d, 1H); 4.70 (m, 1H); 4.55 (m, 2H); 3.85 (m, 1H); 3.60 (m, 3H); 3.20 (m, 5H); 2.96 (s, 3H); 2.40 (m, 2H); 2.00 (m, 2H).

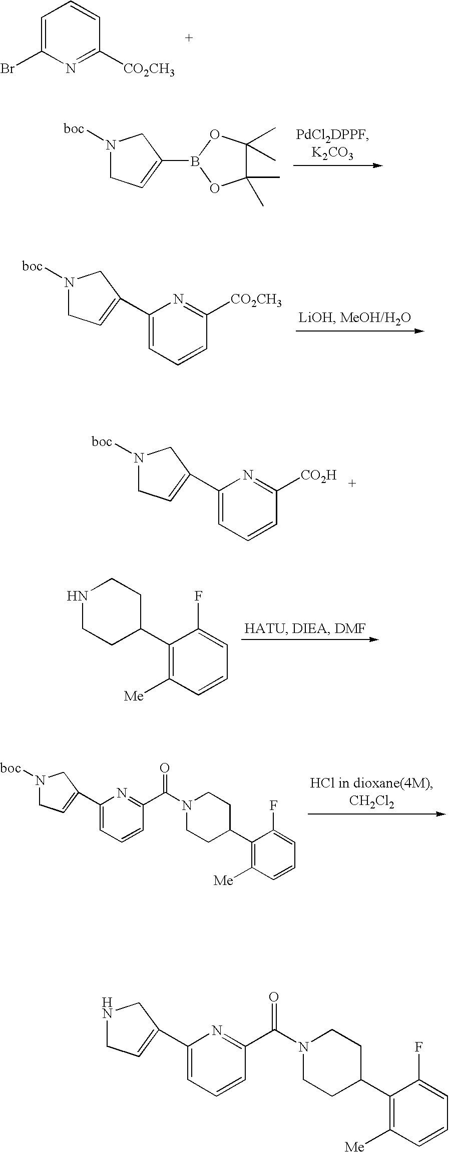

### EXAMPLE 15

### 6-(1-tert-Butoxycarbonyl-2,5-dihydro-1H-pyrrol-3-yl)-pyridine-2-carboxylic acid methyl ester

To a solution of 2,5-dihydro-3-(4,4,5,5-tetramethyl-1,3,2-dioxaborolan-2-yl)-1H-pyrrole-1-carboxylic acid, tert-butyl ester (0.840 g, 2.85 mmol) in DMF (20 mL), nitrogen was bubbled, then 6-bromo-pyridine-2-carboxylic acid methyl ester (0.800 g, 3.70 mmol), potassium carbonate (1.33 g, 9.64 mmol) and [1,1′-bis(diphenylphosphino)ferrocene]dichloropalladium(II) (0.233 g, 0.285 mmol) were added and the whole heated at 100° C. for 2.5 h. The reaction mixture was cooled to room temperature, diluted with EtOAc (50 mL), washed with water, brine, concentrated and the crude material purified by flash chromatography using a gradient 20% to 30% EtOAc in hexane to give 6-(1-tert-butoxycarbonyl-2,5-dihydro-1H-pyrrol-3-yl)-pyridine-2-carboxylic acid methyl ester as a white solid (700 mg, 80.5%). ¹H NMR (400 MHz, CDCl₃) δ: 8.00 (dd, 1H), 7.82 (t, 1H), 7.54 & 7.26 (d, 1H, rotamers), 6.70 & 6.63 (t, 1H, rotamers), 4.62 (m, 2H), 4.38 (m, 2H), 4.00 & 3.99 (s, 3H, rotamers), 1.52 & 1.51 (s, 9H, rotamers).

### 6-(1-tert-Butoxycarbonyl-2,5-dihydro-1H-pyrrol-3-yl)-pyridine-2-carboxylic acid

To a solution of 6-(1-tert-butoxycarbonyl-2,5-dihydro-1H-pyrrol-3-yl)-pyridine-2-carboxylic acid methyl ester (0.70 g, 2.30 mmol) in methanol (11 mL), water (35 mL) and LiOH.H₂O (0.289 g, 6.90 mmol) were added and stirred for 1 h. Methanol was removed under reduced pressure and the aqueous suspension was then acidified with hydrochloric acid to adjust the pH ˜4-5. The precipitate was filtered and dried under vacuum to afford 6-(1-tert-butoxycarbonyl-2,5-dihydro-1H-pyrrol-3-yl)-pyridine-2-carboxylic acid (550 mg, 82%) as an off white solid. ¹H NMR (400 MHz, CD₃OD) δ: 7.91-7.74 (m, 2H), 7.60 & 7.49 (d, 1H, rotamers), 6.76 & 6.68 (t, 1H, rotamers), 4.62 (m, 2H), 4.31 (m, 2H), 1.51 (s, 9H); MS (ES) 291.1 [C₁₅H₁₈N₂O₄+1]⁺.

### 3-{6-[4-(2-Fluoro-6-methyl-phenyl)-piperidine-1-carbonyl]-pyridin-2-yl}-2,5-dihydro-pyrrole-1-carboxylic acid tert-butyl ester

To a solution of 6-(1-tert-butoxycarbonyl-2,5-dihydro-1H-pyrrol-3-yl)-pyridine-2-carboxylic acid (200 mg, 0.689 mmol) in anhydrous DMF (6 mL), 4-(2-fluoro-6-methyl-phenyl)-piperidine (158 mg, 0.689 mmol), O-(7-azabenzotriazol-1-yl N,N,N′,N′-tetramethyluronium hexafluorophosphate (HATU) (367 mg, 0.965 mmol) and Hunigs base (480 μL, 2.76 mmol) were added and stirred at room temperature overnight when completion of the reaction was observed by TLC. The volatiles were removed under vacuum. The residue was diluted with water and extracted with ethyl acetate (2×25 mL). The organic layer was washed with water (5×20 mL), brine (20 mL), dried, concentrated and flash chromatographed using a gradient 25% to 40% ethyl acetate in hexane to furnish 3-{6-[4-(2-fluoro-6-methyl-phenyl)-piperidine-1-carbonyl]-pyridin-2-yl}-2,5-dihydro-pyrrole-1-carboxylic acid tert-butyl ester as a colorless oil (280 mg, 91%). ¹H NMR (400 MHz, CDCl₃) δ: 7.78 (t, 1H), 7.60 (dd, 1H), 7.49 & 7.34 (d, 1H, rotamers), 7.07 (dt, 1H), 6.94 (d, 1H), 6.86 (m, 1H), 6.56 & 6.52 (t, 1H, rotamers), 4.94 (m, 1H), 4.65-4.45 (m, 2H), 4.45-4.20 (m, 3H), 3.25-3.00 (m, 2H), 2.85 (t, 1H), 2.45-2.20 (m, 5H), 1.80 (m, 1H), 1.70 (m, 1H), 1.58 & 1.50 (s, 9H, rotamers); ¹⁹F NMR (400 MHz, CDCl₃) δ: −114; MS (ES) 466.2 [C₂₇H₃₂FN₃O₃+H]⁺.

### [6-2,5-Dihydro-1H-pyrrol-3-yl)-pyridin-2-yl]-[4-2-fluoro-6-methyl-phenyl)-piperidin-1-yl]-methanone

To a solution of compound 11 (240 mg, 0.538 mmol) in anhydrous dichloromethane (10 mL) at 0° C., hydrochloric acid (1.0 mL, 1.00 mmol, 4M solution in dioxane) was added and stirred for 1.5 h. The precipitate was filtered, washed with diethyl ether and dried to give compound [6-(2,5-dihydro-1H-pyrrol-3-yl)-pyridin-2-yl]-[4-(2-fluoro-6-methyl-phenyl)-piperidin-1-yl]-methanone (161 mg, 66%) as a white solid. ¹H NMR (400 MHz, CD₃OD) δ: 7.98 (t, 1H), 7.87 (m, 1H), 7.56 (m, 1H), 7.09 (dt, 1H), 6.98 (d, 1H), 6.85 (dd, 1H), 6.73 (m, 1H), 4.80 (m, 2H), 4.57 (m, 1H), 4.35 (d, 2H), 3.91 (m, 1H), 3.25 (m, 2H), 2.97 (m, 1H), 2.41 (s, 3H), 2.22 (m, 2H), 1.83 (d, 1H), 1.68 (d, 1H); ¹⁰F NMR (400 MHz, CDCl₃) δ: −116; MS (ES) 366.1 [C₂₂H₂₄FN₃O+H]⁺; HPLC purity (98.55%).

The following compounds can be made according to the methods described above.

| | | | | | 5HT6 |
|---|---|---|---|---|---|
| | | IUPAC | | | % inh @ |
| Ex | Method | name | Structure | Analytical | 1 uM |
| 16 | Ex 13 | [4-(3-Fluoro- phenoxy)- piperidin-1- yl]-[6-(1- methyl- pyrrolidin-3- yl)-pyridin-2- yl]- methanone | 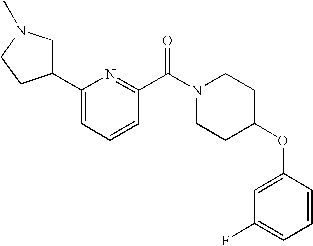 | MS (ES⁺) m/z 384.15 (M + 1), mp = 85-86° C. ¹H NMR (400 MHz, DMSO- d₆): δ 11.30 (br s, 1H), 10.98 (br s, 1H), 8.06-7.99 (m, 1H), 7.60- 7.50 (m, 2H), 7.41-7.30 (m, 1H), 7.00-6.79 (m, 3H), 4.82- 4.78 (m, 1H), 4.18-3.20 (m, 9H), 2.98 (s, 3H), 2.50-1.82 (6H). | 93.2 |
| 17 | Ex 13 | [6-(1-Methyl- pyrrolidin-3- yl)-pyridin-2- yl]-(4-m- tolyloxy- piperidin-1- yl)- methanone | 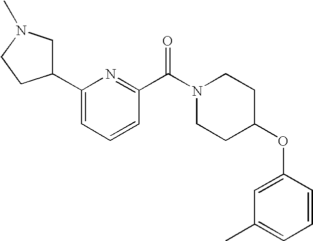 | MS (ES⁺) m/z 380.18 (M + 1), mp = 89-90° C. ¹H NMR (400 MHz, DMSO- d₆): δ 11.10 (br s, 1H), 8.06-7.99 (m, 1H), 7.60- 7.50 (m, 2H), 7.30-7.18 (m, 1H), 6.98-6.79 (m, 3H), 4.80- 4.70 (m, 1H), 4.10-3.10 (m, 9H), 2.98 (s, 3H), 2.70-1.60 (m, 9H). | 96.4 |
| 18 | Ex 12 | [4-(2-Fluoro- phenoxy)- piperidin-1- yl]-[1′- methyl- 1′,2′,3′,6′- tetrahydro- [2,4′]bipyridinyl- 6-yl)- methanone | 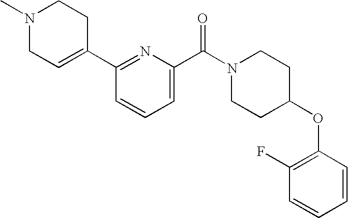 | MS (ES) 396.06 [C₂₃H₂₆FN₃O₂ + H]⁺; ¹H NMR (400 MHz, CD₃OD) δ 7.96 (m, 1H), 7.75 (d, 1H), 7.55 (d, 1H), 7.20-7.06 (m, 3H), 6.96 (m, 1H), 6.74 (m, 1H), 4.68 (m, 1H), 4.13 (m, 1H), 4.00 (m, 1H), 3.88 (m, 1H), 3.78 (m, 3H), 3.48 (m, 1H), 3.36 (m, 1H), 3.16-2.90 (m, 5H), 2.08 (m, 2H), 1.90 (m, 2H); ¹⁹F NMR (400 MHz, CD₃OD) δ −136. | 95.3 |
| 19 | Ex 2 | [6-(4-Methyl- [1,4]diazepan- 1-yl)- pryidin-2-yl]- ((R)-3-m- tolyloxy- pyrrolidin-1- yl)- methanone | 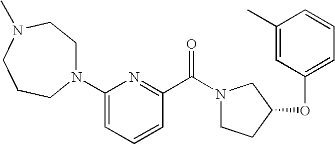 | MS m/z 395 [C₂₃H₃₀N₄O₂ + 1], mp = 82-84° C. ¹H NMR (400 MHz, DMSO- d₆): δ 7.68-7.64 (dd, 1H), 7.19- 7.15 (m, 1H), 7.07-7.04 (d, 1H), 6.81-6.74 (m, 4H), 5.06 (br s, 1H), 3.97-3.36 (m, 10H), 3.21- 3.02 (m, 2H), 2.79-2.74 (2s, 3H), 2.28-2.25 (2s, 3H), 2.28- 2.12 (m, 4H). | 78.5 |
| 20 | Ex 1 | [4-(2-Fluoro- phenyl)- piperidin-1- yl]-[6-(4- methyl- piperazin-1- yl)-pyridin-2- yl]- methanone | 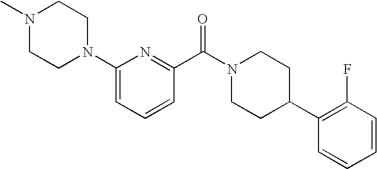 | mp = 111-113° C. ¹H NMR 400 MHz (CDCl₃) δ 7.57 (m, 1H), 7.18 (m, 2H), 7.09 (m, 1H), 7.01 (m, 1H), 6.93 (d, J = 7.8 Hz, 1H), 6.67 (d, J = 8.2 Hz, 1H), 4.87 (m, 1H), 4.03 (br d, 1H), 3.74 (m, 4H), 3.12 (m, 2H), 2.84 (m, 5H), 2.52 (s, 3H), 1.94 (m, 1H), 1.77 (m, 3H). | 101 |
| 21 | Ex 2 | [4-(2-Fluoro- phenyl)-3,6- dihydro-2H- pyridin-1-yl]- [6-(4-methyl- piperazin-1- yl)-pyridin-2- yl]- methanone | 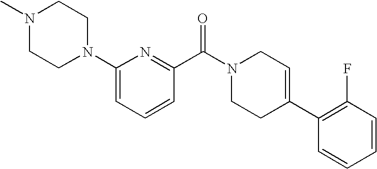 | MS m/z 381 (M + 1), mp 176.7- 177.2° C. ¹H NMR 400 MHz (CD₃OD) δ 7.80 (t, 1H), 7.35- 7.25 (m, 2H), 7.15 (t, 1H), 7.10-7.00 (m, 3H), 6.05-5.90 (m, 1H), 4.55 (d, 2H), 4.35-4.20 (m, 2H), 3.95 (t, 1H), 3.70 (t, 1H), 3.60 (d, 2H), 3.25 (d, 2H), 3.22 (d, 2H), 2.95 (s, 3H), 2.68-2.60 (m, 2H). | 97.8 |
| 22 | Ex 3 | [4-(2-Fluoro- phenyl)- piperidin-1- yl]-(6- piperazin-1- yl-pyridin-2- yl)- methanone | 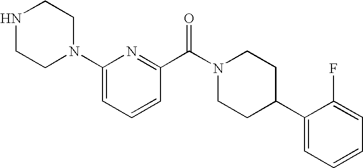 | ¹H NMR (400 MHz, METHANOL-d4) δ ppm 1.50-2.20 (m, 5H), 2.20- 2.40 (m, 1H) 2.60-3.30 (m, 6H) 3.49 (m, 4H) 4.12 (m, 1H) 4.86 (m, 1H) 6.62 (m, 1H) 6.80-7.30 (m, 5H) 7.50 (m, 1H). MS [M + H] = 369. | 96.8 |
| 23 | Ex 1 | [4-(2-Fluoro- 6-methyl- phenyl)- piperidin-1- yl]-[6-(4- methyl- piperazin-1- yl)-pyridin-2- yl]- methanone | 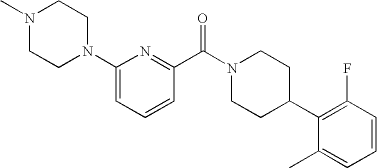 | MS m/z 397 (M + 1), mp = 163- 164.7° C. ¹H NMR 400 MHz (CD₃OD) δ 7.75 (t, 1H), 7.10 (m, 1H), 7.05 (d, 1H), 7.00-6.90 (m, 2H), 6.88- 6.80 (m, 1H), 4.80 (d, 1H), 4.55 (d, 2H), 3.90 (d, 1H), 3.60 (d, 2H), 3.30-3.10 (m, 6H), 3.00 (s, 3H), 2.95 (m, 1H), 2.40 (s, 3H), 2.25-2.10 (m, 2H), 1.80 (d, 1H), 1.65 (d, 1H). | 98.3 |
| 24 | Ex 2 | [6-(4-Methyl- piperazin-1- yl)-pyridin-2- yl]-(4-o-tolyl- 3,6-dihydro- 2H-pyridin- 1-yl)- methanone | 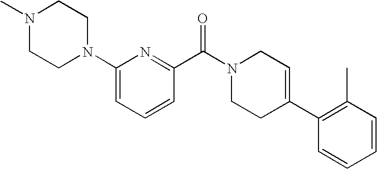 | MS m/z 377 (M + 1), mp 160.7- 162.3° C. ¹H NMR 400 MHz (CD₃OD) δ 7.78 (t, 1H), 7.20- 7.10 (m, 3H), 7.09-7.00 (m, 3H), 5.65-5.50 (m, 1H), 4.58 (d, 2H), 4.32 (m, 1H), 4.20 (m, 1H), 4.00 (t, 1H), 3.70 (t, 1H), 3.60 (d, 2H), 3.30-3.20 (m, 4H), 2.98 (s, 3H), 2.45 (m, 2H), 2.30 (s, 3H). | 92.0 @ 0.3 μM |
| 25 | Ex 1 | (6- [1,4]Diazepan- 1-yl- pyridin-2-yl)- [(S)-3-(4- fluoro- phenoxy)- pyrrolidin-1- yl]- methanone | 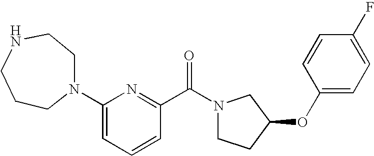 | MS m/z 385 [M +1], mp = 72- 74° C. ¹H NMR (400 MHz, DMSO-d₆): δ 9.00 (br. s, 2H), 7.65 (dd, 1H), 7.14 (m, 2H), 7.02 (m, 2H), 6.96 (dd, 1H), 6.83 (dd, 1H), 5.04 (br. s, 1H), 4.10-3.50 (m, 8H), 3.24-3.13 (m, 4H), 2.21- 1.99 (m, 4H). | 75.4 |
| 26 | Ex 4 | (6- Piperazin-1- yl-pyridin-2- yl)-(2-o-tolyl- piperidin-1- yl)- methanone | 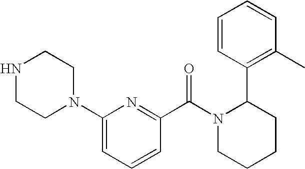 | MS m/z 365 (M + 1), mp = 105.6-106° C. ¹H NMR 400 MHz (CD₃OD) δ 7.75-7.50 (m, 1H), 7.40-7.30 (m, 1H), 7.20- 7.05 (m, 3H), 6.95-6.70 (m, 1H), 5.70 (br s, 1/2H), 5.00 (br s, 1/2H), 3.80- 3.60 (m, 2H), 3.40-3.25 (m, 2H), 3.20-2.90 (m, 6H), 2.40- 2.20 (m, 2H), 1.90 (s, 3H), 1.80-1.40 (m, 4H), 1.10 (s, 1H). | 78.6 |
| 27 | Ex 4 | [2-(3-Fluoro- phenyl)- piperidin-1- yl]-(6- piperazin-1- yl-pyridin-2- yl)- methanone | 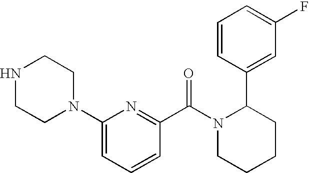 | MS m/z 369 (M + 1), mp = 156.8-157° C. ¹H NMR 400 MHz (CD₃OD) δ 7.80-7.62 (m, 1H), 7.40-7.30 (m, 1H), 7.20- 6.84 (m, 5H), 5.85 (br s, 1/2H), 5.10 (br s 1/2H), 4.55 (d, 1/2H), 3.90- 3.80 (m, 2H), 3.60-3.50 (m, 2H), 3.30 (m, 2H), 3.18-2.95 (m, 3H), 2.75 (t, 1/2H), 2.50- 2.30 (m, 1H), 2.00-1.80 (m, 1H), 1.65-1.40 (m, 4H). | 94.5 |
| 28 | Ex 4 | (6- Piperazin-1- yl-pyridin-2- yl)-(2-m- tolyl- piperidin-1- yl)- methanone | 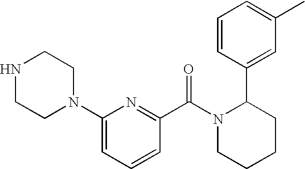 | MS m/z 365 (M + 1), mp = 154.1-155.5° C. ¹H NMR 400 MHz (CD₃OD) δ 7.75-7.60 (m, 1H), 7.20 (t, 1H), 7.15 (s, 1H), 7.10 (d, 1H), 7.00 (d, 1H), 6.90-6.80 (m, 12H), 5.82 (br s, 1/2H), 5.05 (br 2 1/2H), 4.50 (d, 1/2H), 3.80 (m, 2H), 3.52 (d, 1/2H), 3.50- 3.40 (m, 2H), 3.25 (m, 2H), 3.00-2.90 (m, 3H), 2.80 (t, 1H), 2.50-2.30 (m, 1H), 2.25 (s, 3H), 1.98-1.80 (m, 1H), 1.65- 1.40 (m, 4H). | 94.6 |
| 29 | Ex 4 | [2-(4-Fluoro- phenyl)- piperidin-1- yl]-(6- piperazin-1- yl-pyridin-2- yl)- methanone | 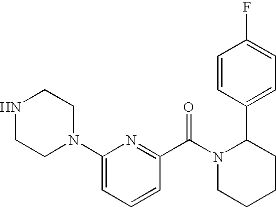 | MS m/z 369 (M + 1), mp = 102- 103.2° C. ¹H NMR 400 MHz (CD₃OD) δ 7.78- 7.60 (m, 1H), 7.35 (t, 2H), 7.10-7.02 (m, 2H), 6.98-6.62 (m, 3H), 5.85 (br s, 1/2H), 5.10 (br s, 1/2H), 4.50 (d, 1/2H), 3.80 (m, 2H), 3.60-3.50 (m, 2H), 3.48 (d, 1/2H), 3.30 (m, 2H), 3.10-2.98 (m, 2H), 2.90- 2.70 (m, 1H), 2.50-2.30 (m, 1H), 2.00-1.80 (m, 1H), 1.70- 1.40 (m, 4H). | 75.1 |
| 30 | Ex 5 | (6- [1,4]Diazepan- 1-yl- pyridin-2-yl)- [(S)-3-(2- fluoro- phenoxy)- pyrrolidin-1- yl]- methanone | 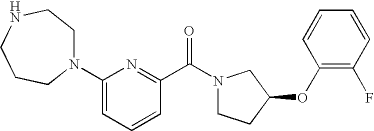 | MS m/z 385 [M + 1], mp = 75- 77° C. ¹H NMR (400 MHz, DMSO-d₆): δ 9.02 (br. s, 2H), 7.64 (dd, 1H), 7.25-7.12 (m, 3H), 7.06-6.92 (m, 2H), 6.82 (dd, 1H), 5.12 (br. s, 1H), 4.10- 3.57 (m, 8H), 3.25-3.06 (m, 4H), 2.21-1.97 (m, 4H). | 98.8 |
| 31 | Ex 4 | (6- [1,4]Diazepan- 1-yl- pyridin-2-yl)- [2-(2-fluoro- phenyl)- piperidin-1- yl]- methanone | 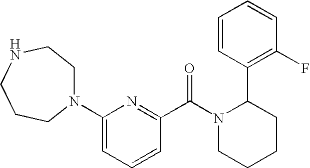 | MS m/z 383 (M + 1), mp. 183.4-184° C. ¹H NMR 400 MHz (CD₃OD) δ 7.80-7.60 (m, 1H), 7.40 (t, 1H), 7.25 (m, 1H), 7.18 (m, 1H), 7.00-6.40 (m, 3H), 5.80 (br s, 1/2H), 5.30 (br s, 1/2H), 4.20- 4.00 (m, 2H), 3.80-3.60 (m, 4H), 3.40-2.98 (m, 4H), 2.30 (d, 1H), 2.20-1.80 (m, 4H), 1.78- 1.40 (m, 4H). | 94.5 |
| 32 | Ex 4 | (6- [1,4]Diazepan- 1-yl- pyridin-2-yl)- [2-(2-fluoro- phenyl)- piperidin-1- yl]- methanone | 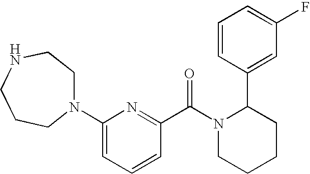 | MS m/z 383 (M + 1), mp = 131.3-134.2° C. ¹H NMR 400 MHz (CD₃OD) δ 7.75-7.60 (m, 1H), 7.40-7.30 (m, 1H), 7.15 (d, 1H), 7.10 (d, 1H), 7.00-6.80 (m, 3H), 5.85 (br s, 1/2H), 5.20 (br s, 1/2H), 4.50 (d, 1/2H), 4.00 (d, 1/2H), 3.80-3.50 (m, 4H), 3.40-3.25 (m, 2H), 3.28- 2.78 (m, 3H), 2.50-2.35 (m, 1H), 2.18 (m, 1H), 2.00-1.80 (m, 2H), 1.70- 1.50 (m, 6H). | 95.0 |
| 33 | Ex 4 | (6- [1,4]Diazepan- 1-yl- pyridin-2-yl)- (2-o-tolyl- piperidin-1- yl)- methanone | 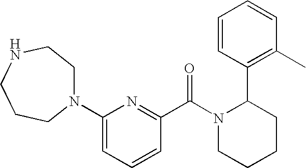 | MS m/z 379 (M + 1), mp = 123.3-124° C. ¹H NMR 400 MHz (CD₃OD) δ 7.70-7.60 (m, 1H), 7.40-7.30 (m, 1H), 7.20- 7.05 (m, 3H), 6.95-6.65 (m, 2H), 5.70 (br s, 1/2H), 4.80 (br s, 1/2H), 4.00- 3.60 (m, 4H), 3.40-3.25 (m, 2H), 3.20-3.10 (m, 2H), 2.40- 2.20 (m, 2H), 2.10 (s, 3H), 2.00-1.80 (m, 7H), 1.25 (m, 2H). | 84.6 |
| 34 | Ex 4 | (6- [1,4]Diazepan- 1-yl- pyridin-2-yl)- (2-m-tolyl- piperidin-1- yl)- methanone | 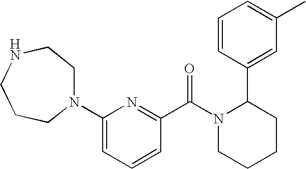 | MS m/z 379 (M + 1), mp = 138.5-139° C. ¹H NMR 400 MHz (CD₃OD) δ 7.80-7.60 (m, 1H), 7.20 (t, 1H), 7.15-7.05 (m, 2H), 7.00 (d, 1H), 6.90-6.80 (m, 2H), 5.82 (br s; 1/2H), 5.18 (br s, 1/2H), 4.50 (d, 1/2H), 4.10-3.98 (m, 1H), 3.80 (m, 2H), 3.70 (d, 1/2H), 2.60- 2.30 (m, 2H), 3.18 (m, 1H), 3.10-2.80 (m, 3H), 2.58-2.40 (m, 1H), 2.35 (s, 3H), 2.20 (m, 1H), 2.10-2.00 (m, 2H), 1.70- 1.50 (m, 5H). | 95.3 |
| 35 | Ex 4 | (6- [1,4]Diazepan- 1-yl- pyridin-2-yl)- [(S)-3-(2- fluoro- phenoxy)- pyrrolidin-1- yl]- methanone | 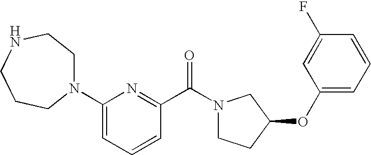 | MS m/z 385 [M + 1], mp = 131-132° C. ¹H NMR (400 MHz, DMSO-d₆): δ 9.05 (br. s, 2H), 7.64 (dd, 1H), 7.32 (dd, 1H), 7.02 (dd, 1H), 6.80 (m, 4H), 5.12 (br. s, 1H), 4.10-3.50 (m, 8H), 3.29-3.07 (m, 4H), 2.29- 1.97 (m, 4H). | 94.4 |
| 36 | Ex 4 | (6- [1,4]Diazepan- 1-yl- pyridin-2-yl)- ((S)-3-o- tolyloxy- pyrrolidin-1- yl)- methanone | 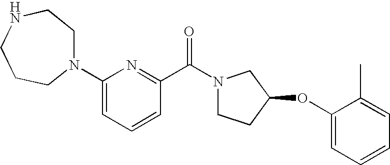 | MS m/z 381 [M + 1], mp = 140-142° C. ¹H NMR (400 MHz, DMSO-d₆): δ 9.02 (br. s, 2H), 7.65 (dd, 1H), 7.19-6.80 (m, 6H), 5.09 (br. s, 1H), 4.13-3.61 (m, 8H), 3.24- 3.15 (m, 4H), 2.21-2.00 (m, 4H), 2.13-2.08 (2s, 3H). | 93.5 |
| 37 | Ex 4 | (6- [1,4]Diazepan- 1-yl- pyridin-2-yl)- (2-p-tolyl- piperidin-1- yl)- methanone | 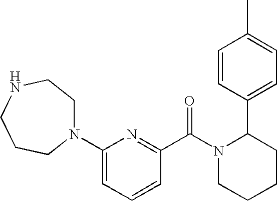 | MS m/z 379 (M + 1), mp = 147.9-148.5° C. ¹H NMR 400 MHz (CD₃OD) δ 7.75-7.60 (m, 1H), 7.20 (d, 2H), 7.15 (d, 3H), 6.95-6.80 (m, 2H), 5.83 (br s, 1/2H), 5.15 (br s. 1/2H) 4.50 (d, 1/2H), 4.00 (m, 1H), 3.78 (m, 1H), 3.65 (d, 1/2H), 3.55 (m, 1H), 3.35-3.25 (m, 1H), 3.15 (m, 1H), 3.10- 2.70 (m, 3H), 2.50-2.30 (m, 1H), 2.22 (s, 3H), 2.18 (m, 1H), 2.00-1.80 (m, 2H), 1.70- 1.40 (m, 6H). | 71.6 |
| 38 | Ex 5 | (6- [1,4]Diazepan- 1-yl- pyridin-2-yl)- [(S)-3-(3- fluoro- phenoxy)- piperidin-1- yl]- methanone | 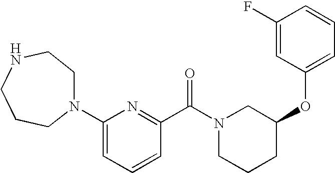 | MS m/z 399 [M + 1], mp = 96.9-97.5° C. ¹H NMR (400 MHz, CD₃OD): δ 7.95-7.71 (m, 1H), 7.30-6.60 (m, 6H), 4.60- 4.50 (m, 1H), 4.15-4.00 (m, 3H), 3.90-3.60 (m, 4H), 3.50- 3.40 (m, 5H), 2.30-1.60 (m, 6H). | 99.4 |
| 39 | Ex 5 | (6- [1,4]Diazepan- 1-yl- pyridin-2-yl)- [(S)-3-(4- fluoro- phenoxy)- piperidin-1- yl]- methanone | 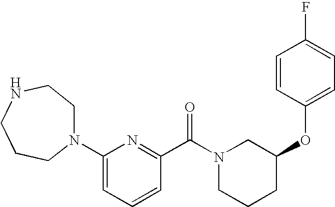 | MS m/z 399 [M + 1], mp = 107.9-109° C. ¹H NMR (400 MHz, CD₃OD): δ 7.75-7.60 (m, 1H), 7.01-6.72 (m, 6H), 4.40- 4.30 (m, 1H), 4.00-3.85 (m, 3H), 3.75-3.55 (m, 4H), 3.45- 3.25 (m, 5H), 2.20-1.50 (m, 6H). | 82.0 |
| 40 | Ex 5 | (6- [1,4]Diazepan- 1-yl- pyridin-2-yl)- [(S)-3-(2- fluoro- phenoxy)- piperidin-1- yl]- methanone | 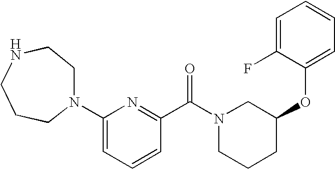 | MS m/z 399 [M + 1], mp = 158- 159° C. ¹H NMR (400 MHz, CD₃OD): δ 7.80- 7.50 (m, 1H), 7.20-6.80 (m, 6H), 4.50-4.35 (m, 1H), 4.05- 3.90 (m, 3H), 3.80-3.60 (m, 4H), 3.50-3.20 (m, 5H), 2.20- 1.40 (m, 6H). | 100 |
| 41 | Ex 5 | (6- [1,4]Diazepan- 1-yl- pyridin-2-yl)- ((S)-3-p- tolyloxy- piperidin-1- yl)- methanone | 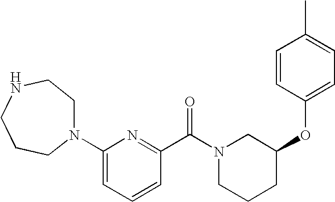 | MS m/z 395 [M + 1], mp = 157.6-158.2° C. ¹H NMR (400 MHz, CD₃OD): δ 7.72- 7.55 (m, 1H), 7.00-6.60 (m, 6H), 4.40-4.30 (m, 1H), 4.00- 3.15 (m, 12H), 2.20-1.50 (m, 9H). | 94.7 |
| 42 | Ex 4 | (6- [1,4]Diazepan- 1-yl- pyridin-2-yl)- ((S)-3-m- tolyloxy- pyrrolidin-1- yl)- methanone | 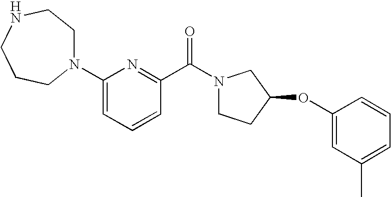 | MS m/z 381 [M + 1], mp = 116-118° C. ¹H NMR (400 MHz, DMSO-d₆): δ 9.05 (br. s, 2H), 7.66 (m, 1H), 7.18 (m, 1H), 7.05 (dd, 1H), 6.89-6.70 (m, 4H), 5.08 (br. s, 1H), 3.99-3.55 (m, 8H), 3.24- 3.05 (m, 4H), 2.25 (2s, 3H), 2.24-1.95 (m, 4H). | 99.4 |
| 43 | Ex 4 | (6- [1,4]Diazepan- 1-yl- pyridin-2-yl)- ((S)-3-p- tolyloxy- pyrrolidin-1- yl)- methanone | 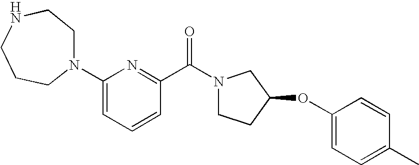 | MS m/z 381 [M + 1], mp = 90- 92° C. ¹H NMR (400 MHz, DMSO-d₆): δ 9.02 (br. s, 2H), 7.70-7.62 (m, 1H), 7.15-7.02 (m, 3H), 6.92- 6.78 (m, 3H), 5.02 (br. s, 1H), 3.98-3.57 (m, 9H), 3.24-3.05 (m, 4H), 2.22 (2s, 3H), 2.22- 1.97 (m, 4H). | 97.7 |
| 44 | Ex 5 | (6- [1,4]Diazepan- 1-yl- pyridin-2-yl)- ((S)-3-m- tolyloxy- piperidin-1- yl)- methanone | 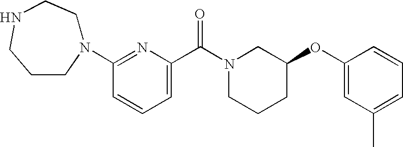 | MS m/z 395 [M + 1], mp = 184- 185.6° C. ¹H NMR (400 MHz, CD₃OD): δ 7.86- 7.60 (m, 1H), 7.18-6.58 (m, 6H), 4.42 (m, 1H), 4.10-3.20 (m, 12H), 2.30- 1.50 (m, 9H). | 99.7 |
| 45 | Ex 5 | (6- [1,4]Diazepan- 1-yl- pyridin-2-yl)- ((S)-3-o- tolyloxy- piperidin-1- yl)- methanone | 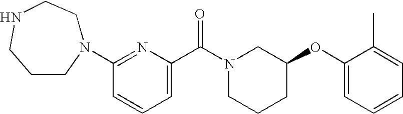 | MS m/z 395 [M + 1], mp = 118- 119.7° C. ¹H NMR (400 MHz, CD₃OD): δ 7.70- 7.48 (m, 1H), 7.08-6.58 (m, 6H), 4.52-4.40 (m, 1H), 4.00- 3.18 (m, 12H), 2.20-1.50 (m, 9H). | 95.4 |
| 46 | Ex 6 | [(R)-3-(2- Fluoro- phenoxy)- pyrrolidin-1- yl]-[6-(4- methyl- [1,4]diazepan- 1-yl)- pyridin-2-yl]- methanone | 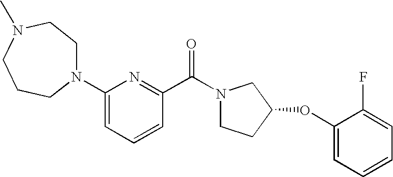 | MS m/z 399 [M + 1], mp = 106-108° C. ¹H NMR (400 MHz, DMSO-d₆): δ 10.58 (br. s, 1H), 7.71-7.65 (dd, 1H), 7.28- 6.94 (m, 5H), 6.85-6.79 (dd, 1H), 5.12 (br. s, 1H), 4.30-3.36 (m, 8H), 3.20- 3.01 (m, 4H), 2.79-2.74 (d, 3H), 2.46-2.16 (m, 4H). | 85.0 |
| 47 | Ex 4 | (6- [1,4]Diazepan- 1-yl- pyridin-2-yl)- [(R)-3-(2- fluoro- phenoxy)- piperidin-1- yl]- methanone | 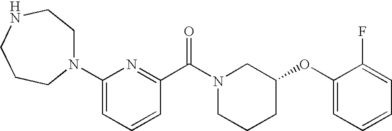 | MS m/z 399 [M + 1], ¹H NMR (400 MHz, CD₃OD): δ 7.80 and 7.65 (2dd, 1H), 7.20-6.89 (m, 6H), 4.60 and 4.48 (2bs, 1H), 4.05 (m, 3H), 3.90-3.70 (m, 4H), 3.60- 3.30 (m, 5H), 2.20 (m, 2H), 2.00 (m, 3H), 1.70-1.55 (m, 1H). | 90.6 |
| 48 | Ex 4 | (6- [1,4]Diazepan- 1-yl- pyridin-2-yl)- ((R)-3-m- tolyloxy- piperidin-1- yl)- methanone | 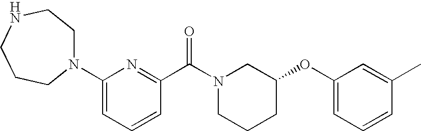 | MS m/z 395 [M + 1], mp = 158- 160° C. ¹H NMR (400 MHz, CD₃OD): δ 7.77 and 7.60 (2dd, 1H), 7.16-6.60 (m, 6H), 4.50 and 4.40 (2bs, 1H), 4.10-3.12 (m, 12H), 2.30 and 2.24 (2s, 2H), 2.22-1.90 (m, 6H). | 86.1 |
| 49 | Ex 4 | (6- [1,4]Diazepan- 1-yl- pyridin-2-yl)- ((R)-3-o- tolyloxy- pyrrolidin-1- yl)- methanone | 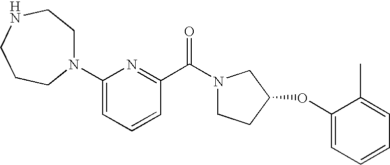 | MS m/z 381 [M + 1], mp = 102-104° C. ¹H NMR (400 MHz, DMSO-d₆): δ 9.00-8.94 (br. s, 2H), 7.67-7.62 (m, 1H), 7.17- 7.12 (m, 2H), 7.03-6.94 (m, 2H), 6.89-6.80 (m, 2H), 5.09 (br. s, 1H), 3.89- 3.61 (m, 8H), 3.25-3.16 (m, 4H), 2.33-1.99 (m, 4H), 2.12- 2.08 (2s, 3H). | 89.9 |
| 50 | Ex 4 | (6- [1,4]Diazepan- 1-yl- pyridin-2-yl)- [(R)-3-(3- fluoro- phenoxy)- piperidin-1- yl]- methanone | 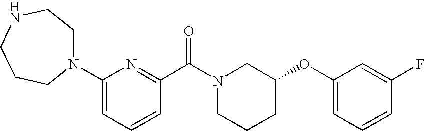 | MS m/z 399 [M + 1], mp = 172-174° C. ¹H NMR (400 MHz, CD₃OD): δ 7.84 and 7.64 (2dd, 1H), 7.28-6.60 (m, 6H), 4.60- 4.50 (m, 1H), 4.10-4.00 (m, 3H), 3.90-3.70 (m, 4H), 3.60- 3.30 (m, 5H), 2.30-1.60 (6H). | 89.6 |
| 51 | Ex 7 | 1-{6-[4-(2- Fluoro- phenyl)- piperidine-1- sulfonyl]- pyridin-2-yl}- piperazine | 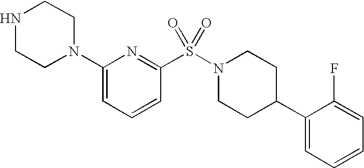 | MS m/z 405 (M + 1), mp. 211- 212.2° C. ¹H NMR 400 MHz (CD₃OD) δ 7.75 (t, 1H), 7.22 (d, 1H), 7.18-7.00 (m, 4H), 6.85 (t, 1H), 3.90 (d, 2H), 3.82-3.75 (m, 4H) 3.30- 3.20 (m, 4H), 2.80-2.70 (m, 3H), 1.80-1.65 (m, 4H). | 97.0 |
| 52 | Ex 4 | [6-(4-Methyl- piperazin-1- yl)-pyridin-2- yl]-((R)-2-m- tolyl- piperidin-1- yl)- methanone | 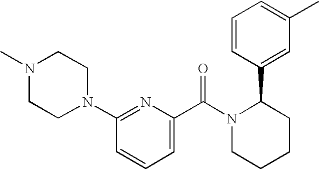 | MS m/z 379 (M + 1), mp = 142- 142.5° C. ¹H NMR 400 MHz (CD₃OD) δ 7.82- 7.70 (m, 1H), 7.25 (t, 1H), 7.20 (t, 2H), 7.10-6.95 (m, 3H), 5.92 (br s, 1/2H), 5.18 (br s, 1/2H), 4.60 (d, 1H), 4.20 (d, 1H), 3.60 (t, 1H), 3.28 (m, 1H), 3.22 (m, 2H), 3.10-2.92 (m, 3H), 2.82 (s, 3H), 2.60-2.40 (m, 1H), 2.38 (s, 3H), 2.00-1.80 (m, 1H), 1.78- 1.50 (m, 5H). | 96.3 |
| 53 | Ex 8 | [6-(4-Methyl- [1,4]diazepan- 1-yl)- pyridin-2-yl]- ((R)-3-o- tolyloxy- pyrrolidin-1- yl)- methanone | 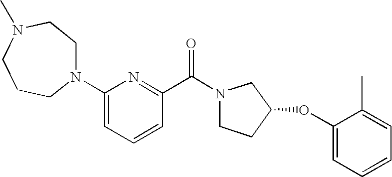 | MS m/z 395 [M + 1], mp = 94- 96° C. ¹H NMR (400 MHz, DMSO-d₆): δ 10.30 (br. s, 1H), 7.70-7.65 (dd, 1H), 7.19- 6.79 (m, 6H), 5.11 (br. s, 1H), 4.00-3.39 (m, 10H), 3.17-3.01 (m, 2H), 2.82- 2.77 (2s, 3H), 2.35-2.09 (m, 4H), 2.14-2.09 (2s, 3H). | 95.9 |
| 54 | Ex 4 | (6- [1,4]Diazepan- 1-yl- pyridin-2-yl)- ((R)-3-p- tolyloxy- piperidin-1- yl)- methanone | 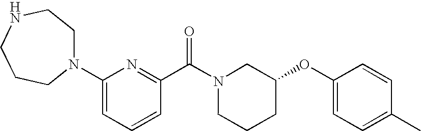 | MS m/z 395 [M + 1], mp = 140° C. ¹H NMR (400 MHz, CD₃OD): δ 7.90 and 7.62 (2dd, 1H), 7.10-6.70 (m, 6H), 4.50- 4.38 (m, 1H), 4.10-3.22 (m, 12H), 2.30 and 2.25 (2s, 3H), 2.24-1.55 (m, 6H). | 86.4 |
| 55 | Ex 4, Ex 8 | [(R)-2-(3- Fluoro- phenyl)- piperidin-1- yl]-[6-(4- methyl- piperazin-1- yl)-pyridin-2- yl]- methanone | 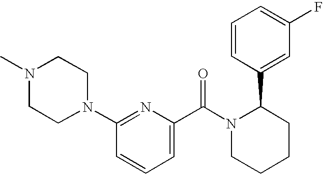 | MS m/z 383 (M + 1), mp = 122.7-123° C. ¹H NMR 400 MHz (CD₃OD) δ 7.80-7.62 (m, 1H), 7.40-7.32 (m, 1H), 7.15 (d, 1H), 7.10 (d, 1H), 7.00-6.90 (m, 3H), 5.82 (br s, 1/2H), 5.10 (br s, 1/2H), 4.58-4.42 (m, 1H), 4.20 (m, 1H), 3.60-3.50 (m, 2H), 3.40- 3.25 (m, 2H), 3.20-3.15 (m, 2H), 3.05-2.90 (m, 2H) 2.88 (s, 3H), 2.80-2.75 (m, 1H), 2.50- 2.30 (m, 1H), 2.00-1.80 (m, 1H), 1.70-1.40 (m, 4H). | 94.2 |
| 56 | Ex 4, Ex 8 | [(S)-2-(2- Fluoro- phenyl)- piperidin-1- yl]-[6-(4- methyl- piperazin-1- yl)-pyridin-2- yl]- methanone | 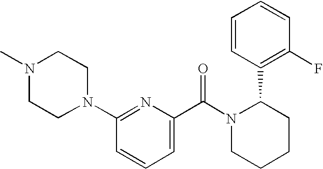 | MS m/z 383 (M + 1), mp = 111.7-112.2° C. ¹H NMR 400 MHz (CD₃OD) δ 7.90-7.70 (m, 1H), 7.45 (t, 1H), 7.30 (m, 1H), 7.20 (m, 1H), 7.10-6.90 (m, 3H), 5.92 (br s, 1/2H), 5.30 (br s, 1/2H), 4.80- 4.42 (m, 1H), 4.10 (m, 1H), 3.60-3.32 (m, 4H), 3.25-3.00 (m, 4H), 2.90 (s, 3H), 2.40-2.20 (m, 1H), 2.10- 1.90 (m, 1H), 1.80-1.50 (m, 4H). | 90.7 |
| 57 | Ex 4, Ex 8 | [(R)-2-(2- Fluoro- phenyl)- piperidin-1- yl]-[6-(4- methyl- piperazin-1- yl)-pyridin-2- yl]- methanone | 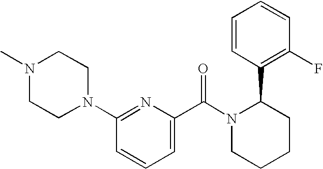 | MS m/z 383 (M + 1), mp = 111.7-112° C. ¹H NMR 400 MHz (CD₃OD) δ 7.80-7.60 (m, 1H), 7.35 (t, 1H), 7.25 (m, 1H), 7.15 (m, 1H), 7.05-6.80 (m, 3H), 5.92 (br s, 1/2H), 5.20 (br s, 1/2H), 4.70- 4.60 (m, 1H), 4.00 (m, 1H), 3.60-3.22 (m, 4H), 3.20-3.00 (m, 4H), 2.82 (s, 3H), 2.30-2.20 (m, 1H), 2.00- 1.80 (m, 1H), 1.78-1.40 (m, 4H. | 94.0 |
| 58 | Ex 2 | [3-(2-Fluoro- phenyl)- piperidin-1- yl]-[6-(4- methyl- piperazin-1- yl)-pyridin-2- yl]- methanone | 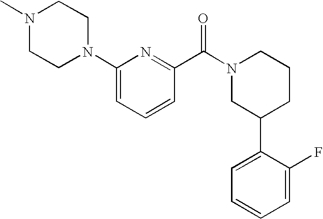 | MS (ES⁺) m/z 383.21 (M + 1), mp = 118-119° C. ¹H NMR (400 MHz, DMSO-d₆): δ 10.99-10.89 (br m, 1H), 7.80- 7.70 (m, 1H), 7.50-6.84 (m, 6H), 4.60-4.28 (m, 3H), 3.80- 3.70 (m, 1H), 3.54-2.70 (m, 12H), 2.00-1.50 (m, 4H). | 88.9 |
| 59 | Ex 4, Ex 8 | [6-(4-Methyl- [1,4]diazepan- 1-yl)- pyridin-2-yl]- ((R)-3-p- tolyloxy- pyrrolidin-1- yl)- methanone | 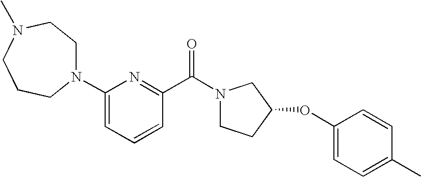 | MS m/z 395 [M + 1], mp = 108-110° C. ¹H NMR (400 MHz, DMSO-d₆): δ 7.68-7.64 (dd, 1H), 7.12-7.03 (m, 3H), 6.88- 6.79 (m, 3H), 5.02 (br. s, 1H), 4.01-3.38 (m, 10H), 3.17-3.01 (m, 2H), 2.81- 2.76 (2s, 3H), 2.24-2.22 (2s, 3H), 2.22-2.12 (m, 4H). | 76.2 |
| 60 | Ex 2 | 6-(4-Methyl- piperazin-1- yl)-pyridine- 2-carboxylic acid methyl- ((R)-1-p- tolyl-ethyl)- amide | 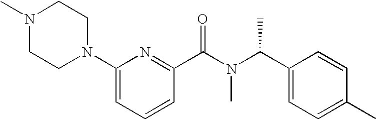 | MS (ES⁺) m/z 383.15 (M + 1), mp = 120-121° C. ¹H NMR (400 MHz, DMSO-d₆): δ 11.30 (br s, 1H), 7.88-7.78 (m, 1H), 7.38-7.20 (m, 4H), 7.18- 6.98 (m, 2H), 5.40-5.00 (m, 1H), 4.50-4.30 (m, 2H), 3.60- 3.22 (m, 4H), 3.20-2.98 (m, 2H), 2.90-2.80 (m, 3H), 2.72- 2.62 (m, 3H), 2.38 (s, 3H), 1.62-1.50 (m, 3H). | 73.8 |
| 61 | Ex 2 | 6-(4-Methyl- piperazin-1- yl)-pyridine- 2-carboxylic acid methyl- ((R)-1-o- tolyl-ethyl)- amide | 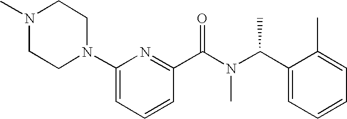 | MS (ES⁺) m/z 353.15 (M + 1), mp = 142-143° C. ¹H NMR (400 MHz, DMSO-d₆): δ 11.10 (br s, 1H), 7.88-7.78 (m, 1H), 7.58-6.90 (m, 6H), 5.98- 5.82 (m, 1H), 4.48-4.30 (m, 2H), 3.60-3.00 (m, 6H), 2.90- 2.30 (m, 9H), 1.78-1.50 (m, 3H). | 86.7 |
| 62 | Ex 2 | [6-(4-Methyl- piperazin-1- yl)-pyridin-2- yl]-((R)-2-o- tolyl- piperidin-1- yl)- methanone | 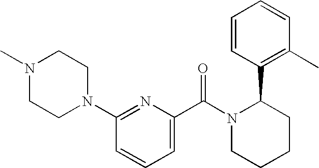 | MS m/z 379 (M + 1), mp = 188- 188.7° C. ¹H NMR 400 MHz (DMSO-d₆) δ 7.78 (m, 1H), 7.40 (m, 1H), 7.20-6.80 (m, 5H), 4.50-4.00 (m, 7H), 3.60- 3.40 (m, 2H), 3.25 (s, 3H), 3.18-2.90 (m, 2H), 2.80 (s, 3H), 2.00-1.50 (m, 6H). | 70.0 |
| 63 | Ex 2 | [6-(4-Methyl- piperazin-1- yl)-pyridin-2- yl]-((S)-2-o- tolyl- piperidin-1- yl)- methanone | 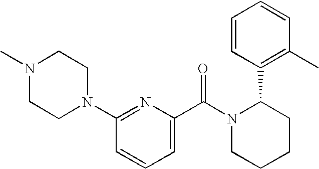 | MS m/z 379 (M + 1), mp = 136.7-137.2° C. ¹H NMR 400 MHz (DMSO- d₆) δ 7.75 (m, 1H), 7.40 (m, 1H), 7.20-6.80 (m, 5H), 4.65- 4.22 (m, 7H), 3.60-3.40 (m, 2H), 3.25 (s, 3H), 3.18-2.90 (m, 2H), 2.80 (s, 3H), 2.00-1.50 (m, 6H). | 77.4 |
| 64 | Ex 2 | 6-(4-Methyl- piperazin-1- yl)-pyridine- 2-carboxylic acid (2- fluoro- benzyl)- methyl- amide | 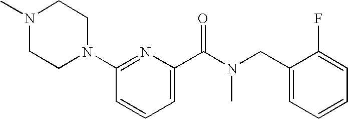 | MS (ES⁺) m/z 343.07 (M + 1), mp = 80-81° C. ¹H NMR (400 MHz, DMSO- d₆): δ 11.38- 11.10 (br m, 1H), 7.80-7.70 (m, 1H), 7.40- 6.90 (m, 6H), 4.78-4.60 (m, 2H), 4.42-4.00 (m, 2H), 3.58- 2.62 (m, 9H), 2.50 (s, 3H). | 87.6 |
| 65 | Ex 2 | 6-(4-Methyl- piperazin-1- yl)-pyridine- 2-carboxylic acid (2- chloro- benzyl)- methyl- amide | 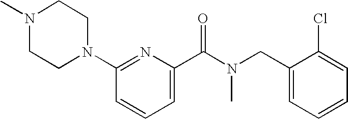 | MS (ES⁺) m/z 359.07 (M + 1), mp = 73-74° C. ¹H NMR (400 MHz, DMSO- d₆): δ 11.00- 10.60 (br m, 1H), 7.80-7.70 (m, 1H), 7.58- 7.30 (m, 4H), 7.10-6.98 (m, 2H), 4.70-4.60 (m, 2H), 4.00- 3.90 (m, 2H), 3.58-2.70 (m, 12H). | 89.6 |
| 66 | Ex 2 | [6-(4-Methyl- piperazin-1- yl)-pyridin-2- yl]-((R)-2-p- tolyl- piperidin-1- yl)- methanone | 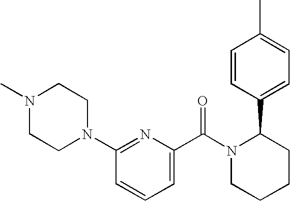 | MS m/z 379 (M + 1), mp = 143.3-144° C. ¹H NMR 400 MHz (DMSO- d₆) δ 7.80-7.60 (m, 1H), 7.22- 7.15 (m, 4H), 7.00-6.82 (m, 2H), 5.80 (br s, 1/2H), 5.00 (br s, 1/2H), 4.80- 4.60 (m, 4H), 4.20 (m, 2H), 4.10 (d, 1H), 3.45 (d, 1H), 3.38-3.22 (m, 1H), 3.20-3.00 (m, 2H), 2.82 (m, 1H), 2.65 (s, 3H), 2.30 (s, 3H), 1.90-1.75 (m, 1H), 1.60 (m, 1H), 1.50- 1.30 (m, 2H). | 82.1 |
| 67 | Ex 2 | [(R)-2-(4- Fluoro- phenyl)- piperidin-1- yl]-[6-(4- methyl- piperazin-1- yl)-pyridin-2- yl]- methanone | 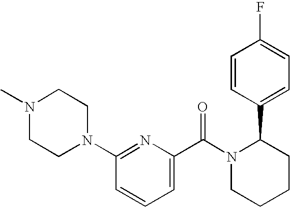 | MS m/z 383 (M + 1), mp = 130.8-131.5° C. ¹H NMR 400 MHz (DMSO- d₆) δ 7.80-7.65 (m, 1H), 7.40 (m, 2H), 7.25- 7.18 (m, 2H), 7.05-6.95 (m, 2H), 5.80 (br s, 1/2H), 5.05 (br s, 1/2H), 4.50- 4.40 (m, 1H), 4.20-4.10 (m, 1H), 3.90-3.70 (m, 6H), 3.50 (d, 1H), 3.30 (m, 1H), 3.20-3.00 (m, 2H), 2.80 (s, 3H), 1.98-1.75 (m, 1H), 1.60 (m, 1H), 1.50- 1.30 (m, 2H). | 79.1 |
| 68 | Ex 2 | [6-(4-Methyl- [1,4]diazepan- 1-yl)- pyridin-2-yl]- ((S)-3-m- tolyloxy- pyrrolidin-1- yl)- methanone | 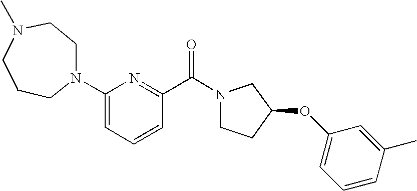 | MS m/z 395 [M + 1], mp = 78- 80° C. ¹H NMR (400 MHz, DMSO-d₆): δ 7.70-7.64 (dd, 1H), 7.21-7.13 (m, 1H), 7.07- 7.054 (d, 1H), 6.84-6.72 (m, 4H), 5.05 (br. s, 1H), 3.98-3.36 (m, 10H), 3.20- 3.05 (m, 2H), 2.78-2.72 (2s, 3H), 2.29-2.25 (2s, 3H), 2.29- 2.13 (m, 4H). | 98.2 |
| 69 | Ex 1 | [(S)-3-(2- Chloro- phenoxy)- pyrrolidin-1- yl]-[6-(4- methyl- [1,4]diazepan- 1-yl)- pyridin-2-yl]- methanone | 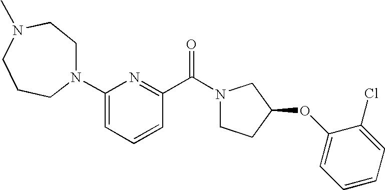 | MS m/z 415 [M + 1], mp = 90- 92° C. ¹H NMR (400 MHz, DMSO-d₆): δ 7.75-7.64 (m, 1H), 7.49-7.20 (m, 3H), 7.12- 6.97 (m, 2H), 6.85-6.78 (m, 1H), 5.19 (br. s, 1H), 4.37-3.38 (m, 10H), 3.21- 3.02 (m, 2H), 2.80-2.78 (2s, 3H), 2.38-2.05 (m, 4H). | 97.7 |
| 70 | Ex 2 | 6-(4-Methyl- piperazin-1- yl)-pyridine- 2-carboxylic acid (3- fluoro- benzyl)- methyl- amide | 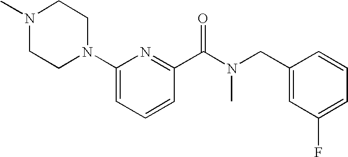 | MS (ES⁺) m/z 343.13 (M + 1), mp = 77-78° C. ¹H NMR (400 MHz, DMSO- d₆): δ 11.10-10- 90 (br m, 1H), 7.80-7.70 (m, 1H), 7.50-7.40 (m, 1H), 7.20- 6.90 (m, 5H), 4.40-4.10 (m, 2H), 3.50-2.70 (m, 14H). | 74.9 |
| 71 | Ex 2 | 6-(4-Methyl- piperazin-1- yl)-pyridine- 2-carboxylic acid (3- chloro- benzyl)- methyl- amide | 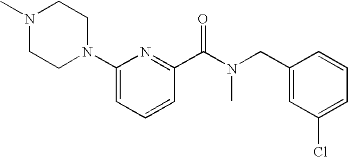 | MS (ES⁺) m/z 359.07 (M + 1), mp = 81-82° C. ¹H NMR (400 MHz, DMSO- d₆): δ 11.80- 11.58 (br m, 1H), 7.80-7.70 (m, 1H), 7.42- 7.20 (m, 4H), 7.10-6.95 (m, 2H), 4.70 (s, 1H), 4.58 (s, 1H), 4.48-4.15 (m, 2H), 3.52- 3.00 (m, 5H), 2.98-2.70 (m, 7H). | 87.1 |
| 72 | Ex 2 | 6-(4-Methyl- piperazin-1- yl)-pyridine- 2-carboxylic acid (2- methoxy- benzyl)- methyl- amide | 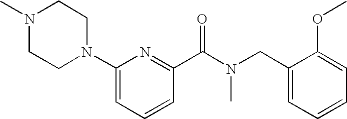 | MS (ES⁺) m/z 355.10 (M + 1), mp = 75-76° C. ¹H NMR (400 MHz, DMSO- d₆): δ 11.10- 10.98 (br m, 1H), 7.80-7.74 (m, 1H), 7.38- 6.90 (m, 6H), 4.61-4.30 (m, 3H), 4.10-3.68 (m, 5H), 3.50- 2.70 (m, 11H). | 89.9 |
| 73 | Ex 2 | 6-(4-Methyl- piperazin-1- yl)-pyridine- 2-carboxylic acid methyl- (3-methyl- benzyl)- amide | 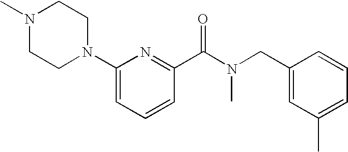 | MS (ES⁺) m/z 339.16 (M + 1), mp = 101-102° C. ¹H NMR (400 MHz, DMSO-d₆): δ 7.80-7.70 (m, 1H), 7.30-7.20 (m, 1H), 7.18- 6.90 (m, 5H), 4.61 (s, 1H), 4.50 (s, 1H), 4.45-4.18 (m, 2H), 3.58-3.00 (m, 5H), 2.98- 2.70 (m, 7H), 2.35 (s, 3H). | 91.9 |
| 74 | Ex 2 | 6-(4-Methyl- piperazin-1- yl)-pyridine- 2-carboxylic acid (2- methoxy- benzyl)- methyl- amide | 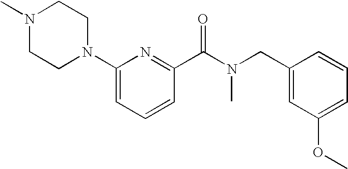 | MS (ES⁺) m/z 355.10 (M + 1), mp = 96-97° C. ¹H NMR (400 MHz, DMSO- d₆): δ 7.80-7.70 (m, 1H), 7.38- 7.22 (m, 1H), 7.10-6.72 (m, 5H), 4.62-4.12 (m, 4H), 3.80- 2.70 (m, 15H). | 85.3 |
| 75 | Ex 1 | [(S)-3-(3- Fluoro- phenoxy)- piperidin-1- yl]-[6-(4- methyl- piperazin-1- yl)-pyridin-2- yl]- methanone | 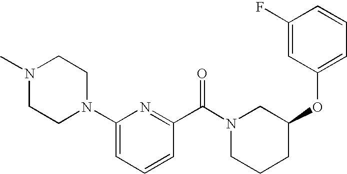 | MS m/z 399 [M + 1], mp = 84- 86° C. ¹H NMR (400 MHz, DMSO-d₆): δ 11.02-10.80 (br. s, 1H), 7.80-7.55 (m, 1H), 7.40- 7.18 (m, 1H), 7.10-6.60 (m, 5H), 4.55-4.05 (m, 3H), 3.80- 2.90 (m, 11H), 2.79-2.74 (2s, 3H), 2.10-1.45 (m, 3H). | 97.3 |
| 76 | Ex 1 | [6-(4-Methyl- piperazin-1- yl)-pyridin-2- yl]-((S)-3-m- tolyloxy- piperidin-1- yl)- methanone | 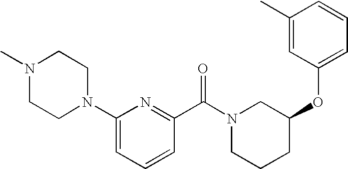 | MS m/z 395 [M + 1], mp = 73- 75° C. ¹H NMR (400 MHz, DMSO-d₆): δ 10.60 (br. s, 1H), 7.80-7.60 (m, 1H), 7.20- 6.55 (m, 6H), 4.50-4.10 (m, 3H), 3.80-2.95 (m, 11H), 2.79- 2.74 (2s, 3H), 2.30-2.22 (2s, 3H), 2.15-1.42 (m, 3H). | 99.0 |
| 77 | Ex 1 | [(S)-3-(3- Chloro- phenoxy)- piperidin-1- yl]-[6-(4- methyl- piperazin-1- yl)-pyridin-2- yl]- methanone | 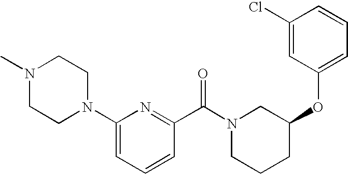 | MS m/z 415 [M + 1], mp = 78- 80° C. ¹H NMR (400 MHz, DMSO-d₆): δ 10.80 (br. s, 1H) 7.80-7.58 (m, 1H), 7.35- 6.75 (m, 6H), 4.60-4.05 (m, 3H), 3.80-2.95 (m, 11H), 2.80- 2.75 (2s, 3H), 2.10-1.45 (m, 3H). | 99.5 |
| 78 | Ex 9 | 1-Methyl-4- [6-(4-o-tolyl- piperidine-1- sulfonyl)- pyridin-2-yl]- piperazine | 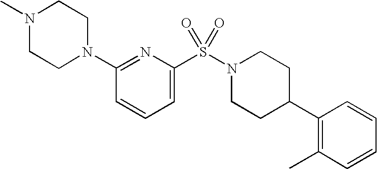 | MS m/z 415 (M + 1), mp = 203- 204.5° C. ¹H NMR (400 MHz, DMSO-d₆) δ 7.88 (t, 1H), 7.25 (t, 2H), 7.18-7.04 (m, 4H), 4.45 (d, 2H), 3.85 (d, 2H), 3.60-3.45 (m, 2H) 3.40- 3.35 (m, 2H), 3.20-3.00 (m, 2H), 2.85-2.75 (m, 6H), 2.25 (s, 3H), 1.75 (d, 2H), 1.70-1.60 (m, 2H). | 98.5 |
| 79 | Ex 9 | 1-{6-[2-(2- Fluoro- phenyl)- piperidine-1- sulfonyl]- pyridin-2-yl}- 4-methyl- piperazine | 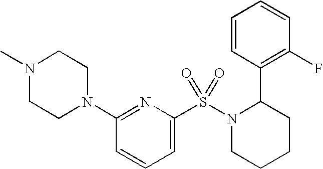 | MS m/z 419 (M + 1), mp = 195.3-196.4° C. ¹H NMR 400 MHz (DMSO- d₆) δ 7.80 (t, 1H), 7.40-7.25 (m, 2H), 7.20- 7.10 (m, 4H), 5.75 (s, 2H), 5.35 (s, 1H), 4.45-4.30 (m, 2H), 3.90 (d, 1H), 3.60-3.40 (m, 2H), 3.18- 3.00 (m, 2H), 2.80 (s, 3H), 1.90 (d, 1H), 1.80-1.42 (m, 3H), 1.40-1.22 (m, 3H). | 90.2 |
| 80 | Ex 2 | [(R)-2-(3- Fluoro- phenyl)- piperidin-1- yl]-[6-(4- methyl- [1,4]diazepan- 1-yl)- pyridin-2-yl]- methanone | 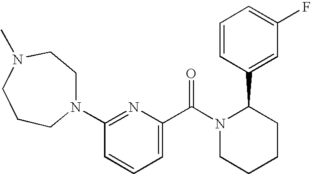 | MS m/z 397 (M + 1), mp = 155- 156.2° C. ¹H NMR 400 MHz (DMSO-d₆) δ 7.80-7.60 (m, 2H), 7.25-7.05 (m, 3H), 6.90- 6.75 (m, 2H), 4.60-4.20 (m, 6H), 3.80-3.30 (m, 6H), 3.05- 3.82 (m. 1H), 2.80 (s, 3H), 2.40-2.20 (m, 2H), 2.00-1.75 (m, 2H), 1.60- 1.30 (m, 2H). | 91.7 |
| 81 | Ex 2 | [(R)-2-(3- Fluoro- phenyl)- piperidin-1- yl]-[6-(4- methyl- [1,4]diazepan- 1-yl)- pyridin-2-yl]- methanone | 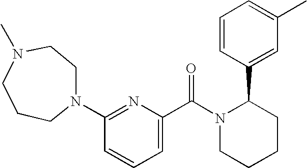 | MS m/z 393 (M + 1), mp = 144.2-143.5° C. ¹H NMR 400 MHz (DMSO- d₆) δ 7.75-7.60 (m, 1H), 7.40- 7.22 (m, 1H), 7.18-7.02 (m, 3H), 6.85-6.65 (m, 2H), 4.60- 4.20 (m, 6H), 3.80-3.22 (m, 4H), 3.20-2.60 (m, 4H), 2.50 (s, 3H), 2.40 (s, 3H), 2.20-1.40 (m, 5H). | 93.6 |
| 82 | Ex 2 | [6-(4-Methyl- piperazin-1- yl)-pyridin-2- yl]-(3-o-tolyl- azetidin-1- yl)- methanone | 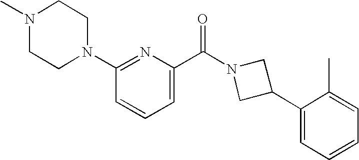 | MS m/z 351 [C₂₁H₂₆N₄O + 1]. ¹H NMR (400 MHz, CDCl₃): δ 7.61 (m, 1H), 7.55 (m, 1H), 7.45 (m, 1H), 7.27 (m, 1H), 7.20 (m, 2H), 6.79 (d, 1H), 5.14 (t, 1H), 4.77 (m, 1H), 4.61 (t, 1H), 4.34 (m, 1H), 4.15 (m, 1H), 3.58 (m, 4H), 2.55 (m, 4H), 2.38 (s, 4H), 2.28 (s, 3H). | 91.3 |
| 83 | Ex 2 | [3-(2-Fluoro- phenyl)- azetidin-1- yl]-[6-(4- methyl- piperazin-1- yl)-pyridin-2- yl]- methanone | 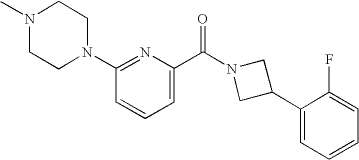 | MS m/z 355 [M + 1]. ¹H NMR (400 MHz, CDCl₃): δ 7.62 (m, 1H), 7.58 (m, 1H), 7.42 (dd, 1H), 7.27 (m, 1H), 7.20 (m, 1H), 7.08 (dd, 1H), 6.79 (d, 1H), 5.18 (t, 1H), 4.78 (m, 1H), 4.61 (t, 1H), 4.36 (m, 1H), 4.18 (m, 1H), 3.55 (m, 4H), 2.55 (m, 4H), 2.32 (s, 3H). | 84.4 |
| 84 | Ex 2 | [3-(3-Fluoro- phenyl)- azetidin-1- yl]-[6-(4- methyl- piperazin-1- yl)-pyridin-2- yl]- methanone | 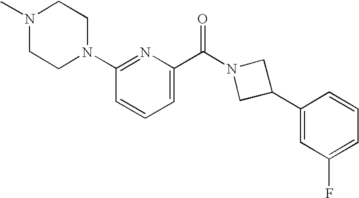 | MS m/z 355 [M + 1]. ¹H NMR (400 MHz, CDCl₃): δ 7.62 (m, 1H), 7.58 (d, 1H), 7.38 (m, 1H), 7.18 (dd, 1H), 7.10 (m, 1H), 6.98 (m, 1H), 6.79 (d, 1H), 5.18 (t, 1H), 4.65 (m, 1H), 4.61 (t, 1H), 4.22 (m, 1H), 3.88 (m, 1H), 3.58 (m, 4H), 2.58 (m, 4H), 2.32 (s, 3H). | 78.0 |
| 85 | Ex 1 | [(S)-3-(3- Fluoro- phenoxy)- piperidin-1- yl]-[6-(4- methyl- [1,4]diazepan- 1-yl)- pyridin-2-yl]- methanone | 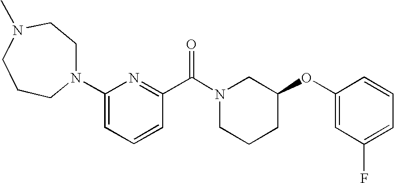 | MS m/z 413 [M + 1], mp = 113-115° C. ¹H NMR (400 MHz, DMSO-d₆): δ 10.45 (br. s, 1H), 7.95-7.50 (m, 1H), 7.40- 7.18 (m, 1H), 7.15-6.65 (m, 5H), 5.00-3.90 (m, 3H), 3.80- 3.30 (m, 8H), 3.25-2.65 (m, 5H), 2.40-1.50 (m, 6H) | 99.1 |
| 86 | Ex 1 | [6-(4-Methyl- [1,4]diazepan- 1-yl)- pyridin-2-yl]- ((S)-3-m- tolyloxy- piperidin-1- yl)- methanone | 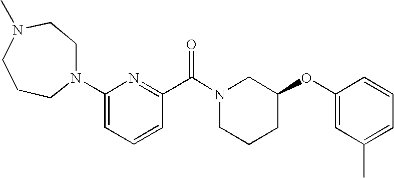 | MS m/z 409 [M + 1], mp = 118-120° C. ¹H NMR (400 MHz, DMSO-d₆): δ 10.35 (br. s, 1H), 7.75-7.55 (m, 1H), 7.23- 7.02 (m, 1H), 6.85-6.55 (m, 5H), 5.00-3.90 (m, 3H), 3.80- 2.95 (m, 8H), 3.80-2.60 (m, 5H), 2.30-2.18 (2s, 3H), 2.30- 1.50 (m, 6H). | 99.5 |
| 87 | Ex 1 | [(S)-3-(3- Chloro- phenoxy)- piperidin-1- yl]-[6-(4- methyl- [1,4]diazepan- 1-yl)- pyridin-2-yl]- methanone | 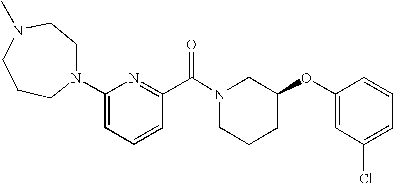 | MS m/z 430 [M + 1], mp = 110-112° C. ¹H NMR (400 MHz, DMSO-d₆): δ 10.50 (br. s, 1H), 7.75-7.50 (m, 1H), 7.40- 7.20 (m, 1H), 7.15-6.70 (m, 5H) 5.20-3.90 (m, 3H), 3.80- 3.25 (m, 8H), 2.80-2.65 (m, 5H), 2.40-1.45 (m, 6H). | 98.9 |
| 88 | Ex 9 | 1-{6-[4-(2- Fluoro- phenyl)- piperidine-1- sulfonyl]- pyridin-2-yl}- 4-methyl- piperazine | 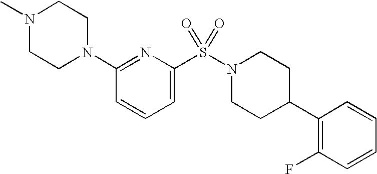 | MS m/z 419 (M + 1), mp = 240.5-242° C. ¹H NMR 400 MHz (DMSO- d₆) δ 7.88 (t, 1H), 7.30 (t, 1H), 7.25 (t, 3H), 7.20-7.12 (m, 2H), 4.40 (d, 2H), 3.85 (d, 2H), 3.60-3.45 (m, 2H) 3.40- 3.35 (m, 2H), 3.20-3.00 (m, 2H), 2.90-2.75 (m, 6H), 1.80- 1.65 (m, 4H). | 102 |
| 89 | Ex 2 | [(R)-2-(2- Fluoro- phenyl)- piperidin-1- yl]-[6-(4- methyl- [1,4]diazepan- 1-yl)- pyridin-2-yl]- methanone | 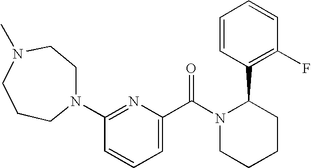 | MS m/z 397 (M + 1), mp = 234.7-135.2° C. ¹H NMR 400 MHz (DMSO- d₆) δ 7.75-7.60 (m, 1H), 7.40- 7.22 (m, 4H), 6.80-6.62 (m, 2H), 4.18-3.82 (m, 6H), 3.75- 3.55 (m, 2H), 3.45-3.25 (m, 2H), 3.18 (m, 1H), 2.80-2.65 (m, 2H), 2.52 (s, 3H), 2.40-2.20 (m, 2H), 2.00- 1.80 (m, 1H), 1.70-1.40 (m, 3H). | 92.3 |
| 90 | Ex 2 | 6-(4-Methyl- piperazin-1- yl)-pyridine- 2-carboxylic acid methyl- (4-methyl- benzyl)- amide | 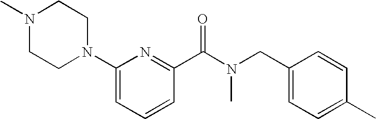 | MS (ES⁺) m/z 339.10 (M + 1), mp = 98-99° C. ¹H NMR (400 MHz, DMSO- d₆): δ 11.38- 11.10 (br m, 1H), 7.79-7.67 (m, 1H), 7.24- 7.10 (m, 4H), 7.05-6.90 (m, 2H), 4.61 (s, 1H), 4.50 (s, 1H), 4.40-4.34 (m, 1H), 4.24- 4.18 (m, 1H), 3.52-3.00 (m, 5H), 2.98-2.70 (m, 7H), 2.30 (s, 3H). | 89.4 |
| 91 | Ex 2 | [6-(4-Methyl- piperazin-1- yl)-pyridin-2- yl]-(3-m- tolyl- azetidin-1- yl)- methanone | 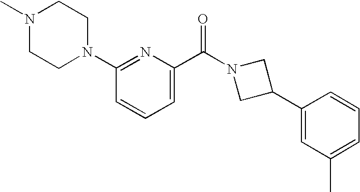 | MS m/z 351 [M + 1]. ¹H NMR (400 MHz, CDCl₃): δ 7.64- 7.55 (m, 2H), 7.32-7.18 (m, 3H), 7.15-7.10 (dd, 1H), 6.88- 6.85 (dd, 1H), 5.18-5.10 (m, 1H), 4.75-4.65 (m, 1H), 4.62- 4.58 (m, 1H), 4.30-4.25 (m, 1H), 3.90-3.80 (m, 1H), 3.58- 3.50 (m, 4H), 2.55-2.50 (m, 4H), 2.38 (s, 3H), 2.36 (s, 3H). | 84.8 |
| 92 | Ex 2 | 6-(4-Methyl- piperazin-1- yl)-pyridine- 2-carboxylic acid methyl- (2- trifluoromethyl- benzyl)- amide | 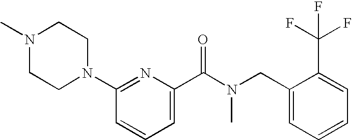 | MS m/z 393 (M + 1), mp = 199.5-200.6° C. ¹H NMR 400 MHz (DMSO- d₆) δ 7.80-7.68 (m, 3H), 7.60- 7.48 (m, 2H), 7.10-6.95 (m, 2H), 4.80 (d, 2H), 4.42 (d, 1H), 4.10 (s, 3H), 3.88 (d, 1H), 3.50 (d, 1H), 3.30 (t, 1H), 3.18 (d, 1H), 2.92 (d, 1H), 2.80 (m, 3H), 2.70 (d, 2H). | 83.0 |
| 93 | Ex 2 | 6-(4-Methyl- piperazin-1- yl)-pyridine- 2-carboxylic acid methyl- ((R)-1-m- tolyl-ethyl)- amide | 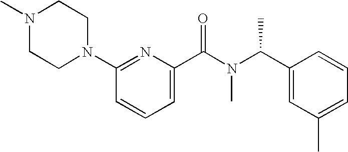 | MS m/z 353 [M + 1], mp = 126-128° C. ¹H NMR (400 MHz, DMSO-d₆): δ 7.80-7.75 (m, 1H), 7.30-7.25 (m, 1H), 7.20- 6.90 (m, 5H), 5.85 & 4.96 (2q, 1H, rotamers), 4.40-4.25 (m, 2H, rotamers), 3.55-3.30 (m, 2H, rotamers), 3.25-2.90 (m, 4H, rotamers), 2.80 (m, 3H, rotamers), 2.60 (m, 3H, rotamers), 2.31 (m, 3H, rotamers), 1.78- 1.50 (m, 3H, rotamers). | 99.4 |
| 94 | Ex 2 | [3-(3-Fluoro- phenyl)- piperidin-1- yl]-[6-(4- methyl- piperazin-1- yl)-pyridin-2- yl]- methanone | 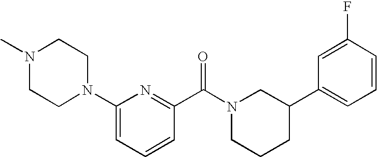 | MS m/z 353 [M + 1], mp = 138-140° C. ¹H NMR (400 MHz, DMSO-d₆): δ 7.78-7.70 (m, 1H), 7.45-7.30 (m, 1H), 7.21- 718 (m, 1H), 7.10-6.90 (m, 3H), 6.85-6.83 (m, 1H), 4.60- 4.30 (m, 3H), 3.78-3.70 (m, 1H), 3.55-3.35 (m, 2H), 3.30- 3.01 (m, 4H), 2.90-2.70 (m, 6H), 1.99-1.48 (m, 4H). | 90.8 |
| 95 | Ex 10 | (1′-Methyl- 1′,2′,3′,4′,5′,6′- hexahydro- [2,4′]bipyridinyl- 6-yl)-(4- o-tolyloxy- piperidin-1- yl)- methanone | 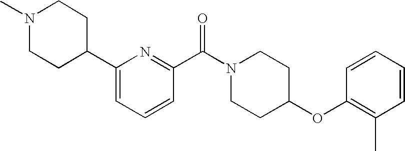 | MS m/z 394 [M + 1], mp = 118-120° C. ¹H NMR (400 MHz, DMSO-d₆): δ 7.91 (dd, 1H), 7.48 (dd, 1H), 7.40 (dd, 1H), 7.15 (m, 2H), 7.00 (dd, 1H), 6.82 (dd, 1H), 4.65 (m, 1H), 3.87 (m, 1H), 3.70-3.47 (m, 4H), 3.39 (m, 1H), 3.16-2.96 (m, 3H), 2.79 (s, 3H), 2.19 (s, 3H), 2.14-1.92 (m, 6H), 1.81- 1.63 (m, 2H). | 84.0 |
| 96 | Ex 10 | [4-(2-Chloro- phenoxy)- piperidin-1- yl]-(1′- methyl- 1′,2′,3′,4′,5′,6′- hexahydro- [2,4′]bipyridinyl- 6-yl)- methanone | 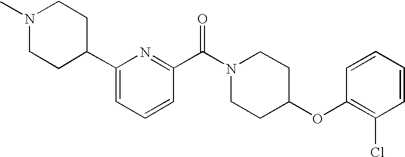 | MS m/z 414 [M + 1], mp = 119-121° C. ¹H NMR (400 MHz, DMSO-d₆): δ 7.91 (dd, 1H), 7.48-7.40 (m, 3H), 7.26 (m, 2H), 6.99 (dd, 1H), 4.80 (m, 1H), 3.88 (m, 1H), 3.62 (m, 2H), 3.55 (m, 2H), 3.39 (m, 1H) 3.15-2.95 (m, 3H), 2.80 (s, 3H), 2.19 (s, 3H), 2.13-1.90 (m, 6H), 1.81- 1.68 (m, 2H). | 85.8 |
| 97 | Ex 11 | [4-(4-Fluoro- phenoxy)- piperidin-1- yl]-[6-(1- methyl- azetidin-3- yl)-pyridin-2- yl]- methanone | 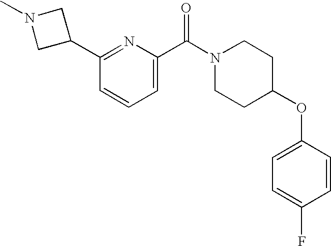 | MS m/z 370 (M + 1), mp = 141.6-142.9° C. ¹H NMR 400 MHz (CD₃OD) δ 7.92 (t, 1H), 7.58 (d, 1H), 7.42 (d, 1H), 7.05-6.90 (m, 4H), 4.70-4.50 (m, 3H), 4.40- 4.20 (m, 3H), 4.00 (m, 1H), 3.80-3.62 (m, 2H), 3.45 (m, 1H), 3.02 (s, 3H), 2.20-2.00 (m, 2H), 1.98- 1.80 (m, 2H). | 79.6 |
| 98 | Ex 12 | (1′-Methyl- 1′,2′,3′,6′- tetrahydro- [2,4′]bipyridinyl- 6-yl)-(4- o-tolyloxy- piperidin-1- yl)- methanone | 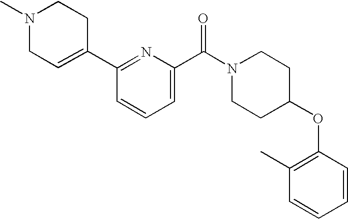 | MS (ES) 392.16 [M + 1]. ¹H NMR (400 MHz, CDCl₃) δ 7.75 (m, 1H), 7.55 (d, 1H), 7.45 (d, 1H), 7.15 (m, 2H), 6.85 (m, 2H), 6.65 (m, 1H), 4.65 (m, 1H), 3.95 (m, 1H), 3.85 (m, 2H), 3.65 (m, 1H), 3.15 (m, 2H), 2.75 (m, 4H), 2.45 (s, 3H), 2.30 (s, 3H), 2.15-1.85 (m, 4H). | 99.1 |
| 99 | Ex 10 | [4-(2-Fluoro- phenoxy)- piperidin-1- yl]-(1′- methyl- 1′,2′,3′,4′,5′,6′- hexahydro- [2,4′]bipyridinyl- 6-yl)- methanone | 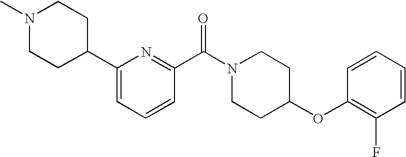 | MS m/z 398 (M + 1). ¹H NMR 400 MHz (CDCl₃) δ 7.72 (t, 1H), 7.45 (d 1H), 7.20 (d, 1H), 7.05-6.90 (m, 4H), 4.60 (m, 1H), 3.98- 3.85 (m, 2H), 3.82-3.75 (m, 1H), 3.58-3.50 (m, 1H), 3.00 (d, 2H), 2.70 (m, 1H), 2.35 (s, 3H), 2.10-2.00 (m, 5H), 1.98- 1.82 (m, 5H). | 78.4 |
| 100 | Ex 12 | [4-(2-Chloro- phenoxy)- piperidin-1- yl]-(1′- methyl- 1′,2′,3′,6′- tetrahydro- [2,4′]bipyridinyl- 6-yl)- methanone | 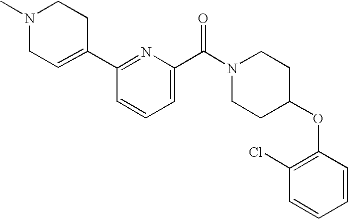 | MS (ES) 412.01 [M + 1]. ¹H NMR (400 MHz, CD₃OD) δ: 7.96 (t, 1H), 7.76 (m, 1H), 7.56 (d, 1H), 7.37 (dd, 1H), 7.25 (m, 1H), 7.15 (dd, 1H), 6.94 (m, 1H), 6.73 (m, 1H), 4.80 (m, 1H), 4.13 (m, 1H), 4.00-3.84 (m, 3H), 3.75 (m, 2H), 3.54 (m, 1H), 3.37 (dt, 1H), 3.10 (m, 1H), 3.02 (s, 3H), 2.96 (m, 1H), 2.20-1.80 (m, 4H). | 102 |
| 101 | Ex 12 | (1′-Methyl- 1′,2′,3′,6′- tetrahydro- [2,4′]bipyridinyl- 6-yl)-[4- (2- trifluoromethyl- phenoxy)- piperidin-1- yl]- methanone | 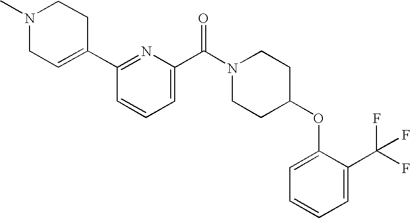 | MS m/z 446.03 [M + 1]. ¹H NMR (400 MHz, CD₃OD) δ 7.97 (t, 1H), 7.76 (d, 1H), 7.57 (m, 3H), 7.24 (d, 1H), 7.05 (t, 1H), 6.73 (m, 1H), 4.95 (m, 1H), 4.15 (m, 1H), 4.00 (m, 1H), 3.90 (m, 1H), 3.85-3.52 (m, 4H), 3.37 (m, 1H), 3.15-2.90 (m, 5H), 2.17- 1.85 (m, 4H); ¹⁹F NMR (400 MHz, CD₃OD) δ -64. | 96.9 |
| 102 | Ex 1 | [4-(2,4- Difluoro- phenyl)- piperidin-1- yl]-[6-(4- methyl- piperazin-1- yl)-pyridin-2- yl]- methanone | 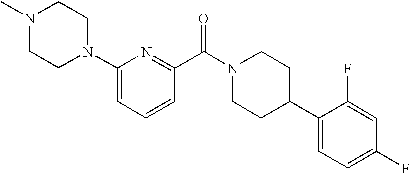 | MS m/z 401 (M + 1), mp = 148.6-149.5° C. ¹H NMR 400 MHz (CD₃OD) δ 7.80 (t, 1H), 7.35 (m, 1H), 7.05 (d, 1H), 7.00 (d, 1H), 6.98-6.85 (m, 2H), 4.80 (d, 1H), 4.55 (d, 2H), 3.95 (d, 1H), 3.60 (d, 2H), 3.30-3.20 (m, 7H), 3.00 (s, 3H), 2.00-1.80 (m, 4H). | 98.7 |
| 103 | Ex 1 | [4-(5-Fluoro- 2-methyl- phenyl)- piperidin-1- yl]-[6-(4- methyl- piperazin-1- yl)-pyridin-2- yl]- methanone | 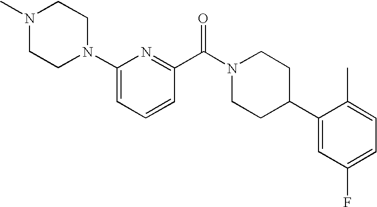 | MS m/z 397 (M + 1). mp = 85.1-86.2° C. ¹H NMR 400 MHz (CD₃OD) δ 7.80 (t, 1H), 7.18 (m, 1H), 7.05 (d, 1H), 7.00 (d, 1H), 6.95 (dd, 1H), 6.80 (m; 1H), 4.80 (d, 1H), 4.60-4.50 (m, 2H), 3.95 (d, 1H), 3.60 (d, 2H), 3.30-3.10 (m, 6H), 3.00 (m, 1H), 2.95 (s, 3H), 2.35 (s, 3H), 1.90 (d, 1H), 1.80-1.62 (m, 3H). | 101 |
| 104 | Ex 1 | [4-(3-Fluoro- 2-methyl- phenyl)- piperidin-1- yl]-[6-(4- methyl- piperazin-1- yl)-pyridin-2- yl]- methanone | 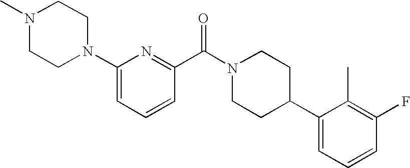 | MS m/z 397 (M + 1), mp = 128.6-129.2° C. ¹H NMR 400 MHz (CD₃OD) δ 7.75 (t, 1H), 7.18 (m, 1H), 7.05 (d, 2H), 7.00 (d, 1H), 6.90 (t, 1H), 4.80 (d, 1H), 4.55 (d, 2H), 3.95 (d, 1H), 3.60 (d, 2H), 3.30-3.10 (m, 6H), 3.00 (m, 1H), 2.95 (s, 3H), 2.50 (s, 3H), 1.90 (d, 1H), 1.80-1.62 (m, 3H). | 101 |
| 105 | Ex 1 | [4-(4-Fluoro- 2-methyl- phenyl)- piperidin-1- yl]-[6-(4- methyl- piperazin-1- yl)-pyridin-2- yl]- methanone | 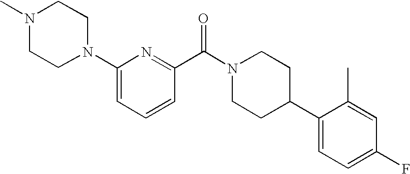 | MS m/z 397 (M + 1), mp = 156.4-157.7° C. ¹H NMR 400 MHz (CD₃OD) δ 7.75 (t, 1H), 7.18 (m, 1H), 7.05 (d, 2H), 7.00 (d, 1H), 6.90 (t, 1H), 4.80 (d, 1H), 4.55 (d, 2H), 3.95 (d, 1H), 3.60 (d, 2H), 3.30-3.10 (m, 6H), 3.00 (m, 1H), 2.95 (s, 3H), 2.5 (s, 3H), 1.90 (d, 1H), 1.80-1.62 (m, 3H). | 97.6 |
| 106 | Ex 13 | [4-(4-Fluoro- 2-methyl- phenyl)- piperidin-1- yl]-[6-(1- methyl- pyrrolidin-3- yl)-pyridin-2- yl]- methanone | 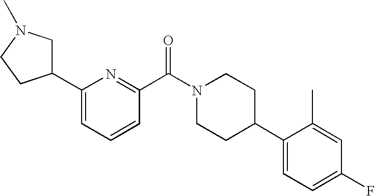 | MS m/z 382 (M + 1), mp = 116.2-117.4° C. ¹H NMR 400 MHz (CD₃OD) δ 8.02-7.92 (m, 1H), 7.60-7.50 (m, 2H), 7.22 (t, 1H), 6.90 (d, 2H), 4.90-4.80 (m, 2H), 4.10- 4.00 (m, 1H), 3.98-3.80 (m, 3H), 3.60-3.50 (m, 1H), 3.38 (m, 1H), 3.20- 3.00 (m, 5H), 2.70-2.50 (m, 1H), 2.40 (s, 3H), 2.25 (m, 1H), 1.95 (d, 1H), 1.80-1.60 (m, 3H). | 93.6 |
| 107 | Ex 14 | [4-Fluoro-4- (2-fluoro- phenyl)- piperidin-1- yl]-[6-(4- methyl- piperazin-1- yl)-pyridin-2- yl]- methanone | 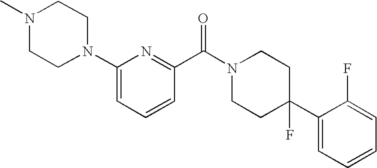 | MS m/n = 401 [MH]⁺. ¹H NMR (400 MHz, CDCl₃): δ 7.81 (dd, 1H); 7.56 (dd, 1H); 7.40- 7.10 (m, 4H); 7.03 (d, 1H); 4.70 (m, 1H); 4.55 (m, 2H); 3.85 (m, 1H); 3.60 (m, 3H); 3.20 (m, 5H); 2.96 (s, 3H); 2.40 (m, 2H); 2.00 (m, 2H). | 97.2 |
| 108 | Ex 13 | [4-(2,4- Difluoro- phenyl)- piperidin-1- yl]-[6-(1- methyl- pyrrolidin-3- yl)-pyridin-2- yl]- methanone | 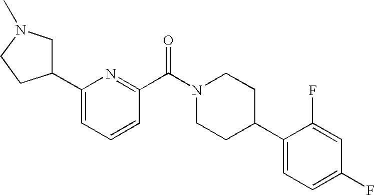 | MS m/z 386 (M + 1), mp = 1176.1-117.9° C. ¹H NMR 400 MHz (CD₃OD) δ 7.95 (t, 1H), 7.55-7.45 (m, 2H), 7.40-7.30 (m, 1H), 6.95- 6.82 (m, 2H), 4.10-4.00 (m, 2H), 3.95-3.78 (m, 3H), 3.50 (m, 1H), 3.30- 3.20 (m, 2H), 3.10-3.00 (m, 4H), 2.78-2.50 (m, 1H), 2.40- 2.20 (m, 1H), 2.00 (m, 1H), 1.90-1.70 (m, 4H). | 95.4 |
| 109 | Ex 2 | [4-(2,4- Difluoro- phenoxy)- piperidin-1- yl]-[6-(4- methyl- piperazin-1- yl)-pyridin-2- yl]- methanone | 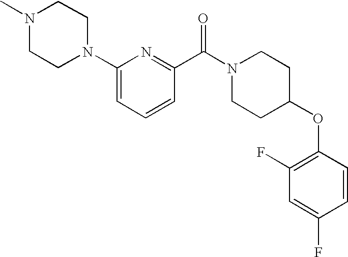 | MS m/z 417.1 [M + 1]. ¹H NMR (400 MHz, CD₃OD) δ 7.93 (m, 1H), 7.26 (d, 1H), 7.19 (m, 1H), 7.07 (d, 1H), 6.99 (m, 1H), 6.88 (m, 1H), 4.60 (m, 1H), 4.52 (m, 2H), 3.96 (m, 1H), 3.76 (m, 2H), 3.66 (d, 2H), 3.49 (m, 3H), 3.26 (m, 2H), 2.96 (s, 3H), 2.04 (m, 2H), 1.85 (m, 2H); ¹⁹F NMR (400 MHz, CD₃OD) δ −120.9, −130.5. | 97.8 |
| 110 | Ex 15 | [6-(2,5- Dihydro-1H- pyrrol-3-yl)- pyridin-2-yl]- [4-(2-fluoro- 6-methyl- phenyl)- piperidin-1- yl]- methanone | 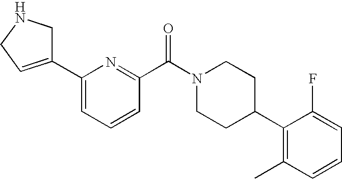 | MS (ES) 366.1 [M + H]. ¹H NMR (400 MHz, CD₃OD) δ 7.98 (t, 1H), 7.87 (m, 1H), 7.56 (m, 1H), 7.09 (dt, 1H), 6.98 (d, 1H), 6.85 (dd, 1H), 6.73 (m, 1H), 4.80 (m, 2H), 4.57 (m, 1H), 4.35 (d, 2H), 3.91 (m, 1H), 3.25 (m, 2H), 2.97 (m, 1H), 2.41 (s, 3H), 2.22 (m, 2H), 1.83 (d, 1H), 1.68 (d, 1H); ¹⁹F NMR (400 MHz, CDCl₃) δ −116. | 98.2 |
| 111 | Ex 13 | [4-(2-Fluoro- 6-methyl- phenyl)- piperidin-1- yl]-[6-(1- methyl- pyrrolidin-3- yl)-pyridin-2- yl]- methanone | 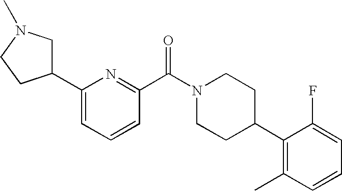 | MS m/z 382 (M + 1), mp = 122.6-123.2° C. ¹H NMR 400 MHz (CD₃OD) δ 7.92 (t, 1H), 7.50-7.45 (m, 2H), 7.10 (m, 1H), 7.00 (d, 1H), 6.90 (m, 1H), 4.85-4.75 (m, 2H), 4.10- 3.80 (m, 3H), 3.50 (m, 1H), 3.38 (m, 1H), 3.30-3.20 (m, 2H), 3.05 (d, 3H), 3.00 (m, 1H), 2.70-2.50 (m, 1H), 2.40 (s, 3H), 2.30-2.10 (m, 3H), 1.95 (d, 1H), 1.70 (d, 1H). | 104 |
| 112 | Ex 13 | [4-(5-Fluoro- 2-methyl- phenyl)- piperidin-1- yl]-[6-(1- methyl- pyrrolidin-3- yl)-pyridin-2- yl]- methanone | 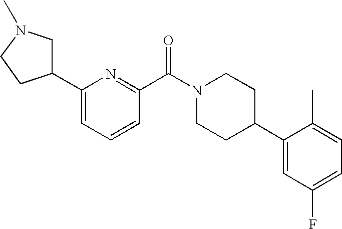 | MS m/z 382 (M + 1), mp = 139.6-1.41° C. ¹H NMR 400 MHz (CD₃OD) δ 7.95 (t, 1H), 7.58-7.45 (m, 2H), 7.18 (t, 1H), 6.90 (d, 1H), 6.80 (m, 1H), 4.90-4.80 (m, 2H), 4.10-4.00 (m, 2H), 3.98- 3.78 (m, 2H), 3.58-3.45 (m, 1H), 3.38 (m, 1H), 3.20-3.00 (m, 5H), 2.70- 2.50 (m, 1H), 2.38 (s, 3H), 2.25 (m, 1H), 1.95 (d, 1H), 1.80-1.60 (m, 3H). | 97.7 |
| 113 | Ex 13 | [4-(2-Fluoro- phenoxy)- piperidin-1- yl]-[6-(1- methyl- pyrrolidin-3- yl)-pyridin-2- yl]- methanone | 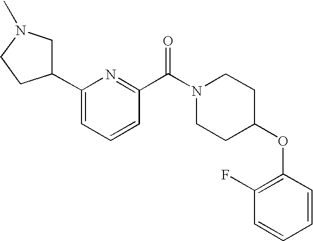 | MS m/z 384 (M + 1), 1H NMR 400 MHz (CDCl₃) δ 7.69 (t, 1H), 7.47 (d, 1H), 7.23-7.26 (m, 1H), 7.00- 7.12 (m, 3H), 6.92-6.98 (m, 1H), 4.58 (s, 1H), 3.92-4.00 (m, 1H), 3.79- 3.90 (m, 2H), 3.51-3.63 (m, 2H), 3.07 (t, 1H), 2.81-2.88 (m, 1H), 2.58-2.66 (m, 2H), 2.41 (s, 3H), 2.30- 2.38 (m, 1H), 1.99-2.10 (m, 4H), 1.90-1.94 (m, 1H). | 97.8 |
| 114 | Ex 3 | [6-(4- Cyclopropyl- piperazin-1- yl)-pyridin-2- yl]-(4-o-tolyl- piperidin-1- yl)- methanone | 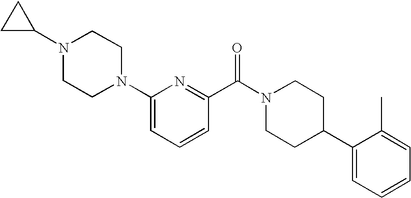 | 1H NMR 400 MHz (CDCl₃) δ 7.56 (t, 1H), 7.12-7.23 (m, 4H), 6.94 (d, 1H), 6.68 (d, 1H), 4.91 (d, 1H), 4.18 (d, 1H), 3.56 (bs, 4H), 3.05-3.17 (m, 1H), 3.02 (m, 1H), 2.88 (m, 1H), 2.81 (m, 1H), 2.73 (bs, 4H), 2.38 (s, 3H), 1.89 (s, 1H), 1.83 (s, 1H), 1.6-1.9 (m, 2H), 0.50 (m, 4H). | 64.5 |
| 115 | Ex 3 | [4-(2-Fluoro- benzyl)- piperidin-1- yl]-[6-(4- methyl- piperazin-1- yl)-pyridin-2- yl]- methanone | 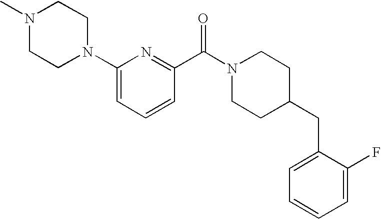 | ¹H NMR 400 MHz (CD₃OD) δ 7.82 (dd, 1H), 7.05-7.19 (m, 5H), 6.96 (d, 1H), 4.47 (m, 3H), 3.75 (m, 1H), 3.61 (m, 2H), 3.37 (m, 2H), 3.21 (m, 1H), 3.05 (m, 1H), 2.95 (s, 3H), 2.82 (m, 1H), 2.63 (d, 2H), 1.91 (m, 1H), 1.78 (m, 1H), 1.62 (m, 1H), 1.30 (m, 3H). | 101 |
| 116 | Ex 1 | [4-(2-Fluoro- phenyl)- azepan-1- yl]-[6-(4- methyl- piperazin-1- yl)-pyridin-2- yl]- methanone | 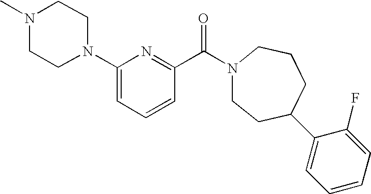 | ¹H NMR 400 MHz (CDCl₃) δ 7.56 (m, 1H), 7.05-7.19 (m, 4H), 6.89 (t, 1H), 6.66 (dd, 1H), 4.12 (m, 1H), 3.93 (m, 1H), 3.49-3.67 (m, 7H), 3.10 (m, 1H), 2.36-2.51 (m, 2H), 2.35 (d, 3H), 2.06 (m, 2H), 1.89 (m, 4H), 1.46 (m, 1H). | 98.5 |

1. A compound formula 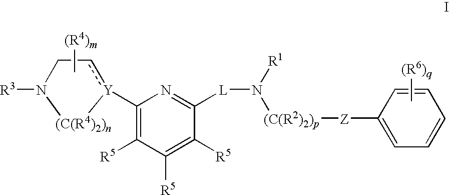
or a pharmaceutically acceptable salt thereof wherein,
L is >C═O, or —SO₂—;
Y is >C(R⁷)— and the dashed line (- - -) is absent; or Y is carbon and the dashed line (- - -) is a double bond; or Y is >N— and the dashed line (- - -) is absent;
Z is a bond, —(C(R²)₂)—, —O—, >C═O, or —S(O)ₜ—; wherein t is an integer selected from 0, 1, or 2;
R¹ is selected from the group consisting of hydrogen, —CF₃, (C₁-C₆)alkyl-, (C₂-C₆)alkenyl-, and (C₂-C₆)alkynyl-; wherein each of aforesaid (C₁-C₆)alkyl-, (C₂-C₆)alkenyl-, and (C₂-C₆)alkynyl- may be optionally substituted by one, two, or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₁-C₉)heterocycloalkyl-, (C₆-C₁₀)aryl-, and (C₁-C₉)heteroaryl-;
each R² is independently selected from the radicals consisting of hydrogen, halo, —CF₃, —CN, —NO₂, —(C═O)R⁸, —(C═O)OR⁹, —O(C═O)R⁸, —OR⁹, —NR¹⁰R¹⁰, —SR¹¹, —(S═O)R¹¹, —SO₂R¹¹, (C₁-C₆)alkyl-, (C₂-C₆)alkenyl-, (C₂-C₆)alkynyl-, (C₃-C₁₀)cycloalkyl-, (C₅-C₁₀)cycloalkenyl-, (C₆-C₁₀)bicycloalkyl-, (C₁-C₉)heterocycloalkyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy, (C₃-C₁₀)cycloalkyl-, (C₁-C₉)heterocycloalkyl-, (C₆-C₁₀)aryl-, and (C₁-C₉)heteroaryl-;
or optionally R¹ and one of said R² may optionally be taken together with the carbon or nitrogen to which they are attached to form an optionally substituted 3 to 10 membered heterocyclic ring optionally containing one or two double or triple bonds and optionally containing one or two additional heteroatoms selected from N, S and O;
or optionally two of said R² may optionally be taken together with the carbon to which they are attached to form an optionally substituted 3 to 10 membered carbocyclic ring optionally containing one or two double or triple bonds; and wherein said 3 to 10 membered carbocyclic ring may optionally contain 1, 2 or 3 heteroatoms independently selected from N, S and O;
R³ is selected from the radicals consisting of hydrogen, —CF₃, —SO₂R¹¹, (C₁-C₆)alkyl-, (C₂-C₆)alkenyl-, (C₂-C₆)alkynyl-, (C₃-C₁₀)cycloalkyl-, (C₅-C₁₀)cycloalkenyl-, (C₆-C₁₀)bicycloalkyl-, (C₆-C₁₀)bicycloalkenyl-, (C₁-C₉)heterocycloalkyl-, (C₂-C₉)heterocycloalkenyl-, (C₂-C₉)heterobicycloalkyl-, (C₂-C₉)heterobicycloalkenyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₁-C₉)heterocycloalkyl-, (C₆-C₁₀)aryl-, and —(C₁-C₉)heteroaryl-;
each R⁴, R⁵ and R⁶ is independently selected from the radicals consisting of hydrogen, halo, —CF₃, —CN, —NO₂, —(C═O)R⁸, —(C═O)OR⁹, —O(C═O)R⁸, —OCF₃, —OR⁹, —NR¹⁰R¹⁰, —(NR¹⁰)(C═O)R⁸, —SR¹¹, —(S═O)R¹¹, —SO₂R¹¹, (C₁-C₆)alkyl-, (C₂-C₆)alkenyl-, (C₂-C₆)alkynyl-, (C₃-C₁₀)cycloalkyl-, (C₅-C₁₀)cycloalkenyl-, (C₆-C₁₀)bicycloalkyl-, (C₆-C₁₀)bicycloalkenyl-, (C₁-C₉)heterocycloalkyl-, (C₂-C₉)heterocycloalkenyl-, (C₂-C₉)heterobicycloalkyl-, (C₂-C₉)heterobicycloalkenyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₁-C₉)heterocycloalkyl-, (C₆-C₁₀)aryl-, and (C₁-C₉)heteroaryl-;
R⁷ is hydrogen, halo, —OR⁹ or (C₁-C₆)alkyl-;
each R⁸ is independently selected from the radicals consisting of hydrogen, (C₁-C₆)alkyl-, (C₂-C₆)alkenyl-, (C₂-C₆)alkynyl-, (C₃-C₁₀)cycloalkyl-, (C₁-C₉)heterocycloalkyl-, (C₆-C₁₀)aryl-, and (C₁-C₉)heteroaryl-; wherein each of the aforesaid radicals may be optionally substituted by one, two or three substituents independently selected from the group consisting of hydrogen, halo, —CF₃, —OCF₃, hydroxyl, amino, (C₁-C₆)alkylamino, di((C₁-C₆)alkyl)amino-, (C₁-C₆)alkyl-, (C₁-C₆)alkoxy-, (C₃-C₁₀)cycloalkyl-, (C₆-C₁₀)aryl-, and (C₁-C₉)heteroaryl-;
each R⁹ is independently selected from the radicals consisting of hydrogen, (C₁-C₆)alkyl-, (C₂-C₆)alkenyl-, (C₂-C₆)alkynyl-, (C₃-C₁₀)cycloalkyl-, (C₁-C₉)heterocycloalkyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₆-C₁₀)aryl-, and (C₁-C₉)heteroaryl-;
each R¹⁰ is independently selected from the radicals consisting of hydrogen, —SO₂—(C₁-C₆)alkyl, (C₁-C₆)alkyl-, (C₂-C₆)alkenyl-, (C₂-C₆)alkynyl-, (C₃-C₁₀)cycloalkyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, (C₁-C₆)alkyl-, (C₁-C₆)alkoxy-, (C₃-C₁₀)cycloalkyl-, (C₅-C₁₀)cycloalkenyl-, (C₆-C₁₀)aryl-, and (C₁-C₉)heteroaryl-;
each R¹¹ is independently selected from the group consisting of hydrogen, amino, (C₁-C₆)alkylamino-, di((C₁-C₆)alkyl)amino-, —CF₃, (C₁-C₆)alkyl-, (C₃-C₁₀)cycloalkyl-, (C₁-C₉)heterocycloalkyl-, (C₆-C₁₀)aryl-, and (C₁-C₉)heteroaryl-;
n is an integer selected from one, two or three;
m an integer selected from zero, one, two, three or four;
p is an integer selected from zero, one, two, three or four; and
q is an integer selected from zero, one, two, three or four.

2. A compound according to claim 1 wherein, Y is —C(R⁷)— and the dashed line (- - -) is absent, or a pharmaceutically acceptable salt thereof.

3. A compound according to claim 2 wherein, Y is —C(R⁷)— and R⁷ is hydrogen or a pharmaceutically acceptable salt thereof.

4. A compound according to claim 2 wherein, Y is —C(R⁷)— and R⁷ is halo or a pharmaceutically acceptable salt thereof.

5. A compound according to claim 1 wherein, Y is >N— and the dashed line (- - -) is absent, or a pharmaceutically acceptable salt thereof.

6. A compound according to claim 5 wherein, m is zero, or a pharmaceutically acceptable salt thereof.

7. A compound according to claim 5 wherein, m is one, or a pharmaceutically acceptable salt thereof.

8. A compound according to claim 6 wherein, R³ is hydrogen, or a pharmaceutically acceptable salt thereof.

9. A compound according to claim 6 wherein, R³ is (C₁-C₆)alkyl optionally substituted with by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₃-C₁₀)cycloalkyl-, (C₁-C₉)heterocycloalkyl-, (C₆-C₁₀)aryl-, and —(C₁-C₉)heteroaryl-, or a pharmaceutically acceptable salt thereof.

10. A compound according to claim 9 wherein said R³ (C₁-C₆)alkyl is methyl optionally substituted with by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl- and (C₁-C₆)alkoxy-, or a pharmaceutically acceptable salt thereof.

11. A compound according to claim 5 wherein L is >C═O, or a pharmaceutically acceptable salt thereof.

12. A compound according to claim 5 wherein L is —SO₂—, or a pharmaceutically acceptable salt thereof.

13. A compound according to claim 5 wherein q is 1 or 2 and each R⁶ is independently selected from the radicals consisting of halo, —CF₃, —CN, —NO₂, —(C═O)R⁸, —(C═O)OR⁹, —O(C═O)R⁸, —OCF₃, —OR⁹, —O(C═O)OR⁹, —NR¹⁰R¹⁰, —(NR¹⁰)(C═O)R⁸, —SR¹¹, —(S═O)R¹¹, —SO₂R¹¹, (C₁-C₆)alkyl-, (C₂-C₆)alkenyl-, (C₂-C₆)alkynyl-, (C₃-C₁₀)cycloalkyl-, (C₆-C₁₀)aryl-, and (C₁-C₉)heteroaryl-; wherein each of the aforesaid radicals may be optionally substituted by one, two or three substituents independently selected from hydrogen, halo, —CF₃, —OCF₃, hydroxyl, amino, (C₁-C₆)alkylamino-, di((C₁-C₆)alkyl)amino-, (C₁-C₆)alkyl-, (C₁-C₆)alkoxy-, (C₂-C₆)alkenyl, (C₂-C₆)alkynyl-, (C₃-C₁₀)cycloalkyl-, (C₅-C₁₀)cycloalkenyl-, (C₆-C₁₀)bicycloalkyl-, (C₆-C₁₀)bicycloalkenyl-, (C₁-C₉)heterocycloalkyl-, (C₂-C₉)heterocycloalkenyl-, (C₂-C₉)heterobicycloalkyl-, (C₂-C₉)heterobicycloalkenyl-, (C₅-C₁₀)aryl, and (C₁-C₉)heteroaryl-, or a pharmaceutically acceptable salt thereof.

14. A compound according to claim 5 wherein Z is a bond, or a pharmaceutically acceptable salt thereof.

15. A compound according to claim 5 wherein Z is —O—, or a pharmaceutically acceptable salt thereof.

16. A compound according to claim 5 wherein R¹ and one of said R² are taken together with the carbon or nitrogen to which they are attached to form an optionally substituted 3 to 10 membered heterocyclic ring optionally containing one or two double or triple bonds and optionally containing one or two additional heteroatoms selected from N, S and O, or a pharmaceutically acceptable salt thereof.

17. A compound according to claim 16 wherein said optionally substituted 3 to 10 membered heterocyclic ring optionally containing one or two double or triple bonds is selected from azetidinyl, pyrrolidinyl, 3-pyrolin-1-yl, piperidinyl, 1,2,3,6-tetrahydropyridin-1-yl, perhydroazepinyl, heptamethyleneinyl, octahydroazoninyl, azabicyclo(2.2.1)heptan-3-one, and tropanyl, or a pharmaceutically acceptable salt thereof.

18. A compound according to claim 5 wherein two of said R² groups are taken together with the carbon to which they are attached to form an optionally substituted 3 to 10 membered carbocyclic ring optionally containing one or two double or triple bonds; and wherein said 3 to 10 membered carbocyclic ring may optionally contain 1, 2 or 3 additional heteroatoms, or a pharmaceutically acceptable salt thereof.

19. A compound according to claim 18 wherein said optionally substituted 3 to 10 membered carbocyclic or heterocyclic ring optionally containing one or two double or triple bonds is selected from cyclopropyl, cyclobutyl, cyclobutenyl, cyclopentyl, cyclopentenyl, cyclohexyl, cyclohexenyl, cyclohexadinenyl, azetidinyl, pyrolidinyl, and piperidinyl, or a pharmaceutically acceptable salt thereof.

20. A compound according to claim 1 wherein said compound is:
(6-Piperazin-1-yl)-pyridin-2-yl)-(4-o-tolyl-piperidin-1-yl)-methanone;
[4-(3-Fluoro-2-methyl-phenyl)-piperidin-1-yl]-[6-(4-methyl-piperazin-1-yl)-pyridin-2-yl]-methanone;
[4-(4-Fluoro-2-methyl-phenyl)-piperidin-1-yl]-[6-(4-methyl-piperazin-1-yl)-pyridin-2-yl]-methanone;
[4-(5-Fluoro-2-methyl-phenyl)-piperidin-1-yl]-[6-(4-methyl-piperazin-1-yl)-pyridin-2-yl]-methanone;
[4-(2-Fluoro-6-methyl-phenyl)-piperidin-1-yl]-[6-(4-methyl-piperazin-1-yl)-pyridin-2-yl]-methanone;
[4-(2-Fluoro-phenyl)-piperidin-1-yl]-[6-(4-methyl-piperazin-1-yl)-pyridin-2-yl]-methanone;
[4-(2,3-Difluoro-phenyl)-piperidin-1-yl]-[6-(4-methyl-piperazin-1-yl)-pyridin-2-yl]-methanone;
[4-(2,4-Difluoro-phenyl)-piperidin-1-yl]-[6-(4-methyl-piperazin-1-yl)-pyridin-2-yl]-methanone;
[4-(2,5-Difluoro-phenyl)-piperidin-1-yl]-[6-(4-methyl-piperazin-1-yl)-pyridin-2-yl]-methanone;
[4-(2,6-Difluoro-phenyl)-piperidin-1-yl]-[6-(4-methyl-piperazin-1-yl)-pyridin-2-yl]-methanone;
[4-Fluoro-4-(2-fluoro-phenyl)-piperidin-1-yl]-[6-(4-methyl-piperazin-1-yl)-pyridin-2-yl]-methanone;
(4-Fluoro-4-o-tolyl-piperidin-1-yl)-[6-(4-methyl-piperazin-1-yl)-pyridin-2-yl]-methanone;
6-(1-Methyl-pyrrolidin-3-yl)-pyridin-2-yl]-(4-o-tolyloxy-piperidin-1-yl)-methanone;
[4-(2-Fluoro-phenoxy)-piperidin-1-yl]-(6-pyrrolidin-3-yl-pyridin-2-yl)-methanone;
[4-(2-Chloro-phenoxy)-piperidin-1-yl]-[6-(1-methyl-pyrrolidin-3-yl)-pyridin-2-yl]-methanone; or
[6-(1-Methyl-pyrrolidin-3-yl)-pyridin-2-yl]-[4-(2-trifluoromethyl-phenoxy)-piperidin-1-yl]-methanone;
or a pharmaceutically acceptable salt of each of the foregoing compounds.

21. A compound according to claim 1 wherein said compound is:
[4-(4-Fluoro-2-methyl-phenoxy)-piperidin-1-yl]-[6-(1-methyl-pyrrolidin-3-yl)-pyridin-2-yl]-methanone;
[4-(2,5-Difluoro-phenoxy)-piperidin-1-yl]-[6-(1-methyl-pyrrolidin-3-yl)-pyridin-2-yl]-methanone;
[4-(2,4-Difluoro-phenyl)-piperidin-1-yl]-[6-(1-methyl-pyrrolidin-3-yl)-pyridin-2-yl]-methanone;
[4-(4-Fluoro-2-methyl-phenyl)-piperidin-1-yl]-[6-(1-methyl-pyrrolidin-3-yl)-pyridin-2-yl]-methanone;
[4-(5-Fluoro-2-methyl-phenyl)-piperidin-1-yl]-[6-(1-methyl-pyrrolidin-3-yl)-pyridin-2-yl]-methanone;
[4-(2-Fluoro-6-methyl-phenyl)-piperidin-1-yl]-[6-(1-methyl-pyrrolidin-3-yl)-pyridin-2-yl]-methanone; or
[4-(2-Fluoro-6-methyl-phenyl)-piperidin-1-yl]-[6-(3-fluoro-1-methyl-pyrrolidin-3-yl)-pyridin-2-yl]-methanone;
or a pharmaceutically acceptable salt of each of the foregoing compounds.

22. A method for treating schizophrenia in a mammal comprising administering to a mammal a therapeutically effective amount of a compound of Formula I according to claim 1, or a pharmaceutically acceptable salt thereof.

23. A pharmaceutical composition comprising a therapeutically effective amount of a compound of Formula I, according to claim 1, or a pharmaceutically acceptable salt thereof.
